SEQUENCE LISTING (160 sequences)

SEQ ID NO: 1 - protein, 510 aa, Mus musculus
MDPLQAVHLGPRKKRPRQLGTPVASTPYDIRFRDLVLFILEKKMGTTRRAFLMELARRKGFRVENELSDSVTHIVAENNSGSDVLEWLQLQNIKASSELELLDISWLIECMGAGKPVEMMGRHQLVVNRNSSPSPVPGSQNVPAPAVKKISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKRVFLEAESEEEIFAHLGLDYIEPWERNA

SEQ ID NO: 2 - DNA, 12 bp, Artificial Sequence
  Test Polynucleotide p875
cagttaaaaact

SEQ ID NO: 3 - DNA, 11 bp, Artificial Sequence
  Test Polynucleotide p876
gagttaaaact

SEQ ID NO: 4 - DNA, 10 bp, Artificial Sequence
  Test Polynucleotide p877
cagcaaggct

SEQ ID NO: 5 - DNA, 18 bp, Artificial Sequence
  primer
aaaaaaaaaaaaaagggg

SEQ ID NO: 6 - protein, 25 aa, Artificial Sequence
  affinity tag for purifying TdT variants
MASSHHHHHHSSGSENLYFQTGSSG

SEQ ID NO: 7 - protein, 381 aa, Mus musculus
NSSPSPVPGSQNVPAPAVKKISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKRVFLEAESEEEIFAHLGLDYIEPWERNA

SEQ ID NO: 8 - protein, 380 aa, Bos taurus
DYSATPNPGFQKTPPLAVKKISQYACQRKTTLNNYNHIFTDAFEILAENSEFKENEVSYVTFMRAASVLKSLPFTIISMKDTEGIPCLGDKVKCIIEEIIEDGESSEVKAVLNDERYQSFKLFTSVFGVGLKTSEKWFRMGFRSLSKIMSDKTLKFTKMQKAGFLYYEDLVSCVTRAEAEAVGVLVKEAVWAFLPDAFVTMTGGFRRGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSRQFERDIRRYATHERKMMLDNHALYDKTKRVFLKAESEEEIFAHLGLDYIEPWERNA

SEQ ID NO: 9 - protein, 380 aa, Homo sapiens
DYSDSTNPGPPKTPPIAVQKISQYACQRRTTLNNCNQIFTDAFDILAENCEFRENEDSCVTFMRAASVLKSLPFTIISMKDTEGIPCLGSKVKGIIEEIIEDGESSEVKAVLNDERYQSFKLFTSVFGVGLKTSEKWFRMGFRTLSKVRSDKSLKFTRMQKAGFLYYEDLVSCVTRAEAEAVSVLVKEAVWAFLPDAFVTMTGGFRRGKKMGHDVDFLITSPGSTEDEEQLLQKVMNLWEKKGLLLYYDLVESTFEKLRLPSRKVDALDHFQKCFLIFKLPRQRVDSDQSSWQEGKTWKAIRVDLVLCPYERRAFALLGWTGSRQFERDLRRYATHERKMILDNHALYDKTKRIFLKAESEEEIFAHLGLDYIEPWERNA

SEQ ID NO: 10 - protein, 376 aa, Gallus gallus
QYPTLKTPESEVSSFTASKVSQYSCQRKTTLNNCNKKFTDAFEIMAENYEFKENEIFCLEFLRAASVLKSLPFPVTRMKDIQGLPCMGDRVRDVIEEIIEEGESSRAKDVLNDERYKSFKEFTSVFGVGVKTSEKWFRMGLRTVEEVKADKTLKLSKMQRAGFLYYEDLVSCVSKAEADAVSSIVKNTVCTFLPDALVTITGGFRRGKKIGHDIDFLITSPGQREDDELLHKGLLLYCDIIESTFVKEQIPSRHVDAMDHFQKCFAILKLYQPRVDNSSYNMSKKCDMAEVKDWKAIRVDLVITPFEQYAYALLGWTGSRQFGRDLRRYATHERKMMLDNHALYDKRKRVFLKAGSEEEIFAHLGLDYVEPWERNA

SEQ ID NO: 11 - protein, 387 aa, Monodelphis domestica
SANPDPTAGTLNILPPTTKTISQYACQRRTTINNHNQRFTDAFEILAKNYEFKENDDTCLTFMRAISVLKCLPFEVVSLKDTEGLPWIGDEVKGIMEEIIEDGESLEVQAVLNDERYQSFKLFTSVFGVGLKTADKWYRMGFRTLNKIRSDKTLKLTKMQKAGLCYYEDLIDCVSKAEADAVSLLVQDAVWTFLPDALVTITGGFRRGKEFGHDVDFLITSPGAEKEQEDQLLQKVTNLWKKQGLLLYCDLIESTFEDLKLPSRKIDALDHFQKCFLILKLYHHKEDKRKWEMPTGSNESEAKSWKAIRVDLVVCPYDRYAFALLGWSGSRQFERDLRRYATHEKKMMLDNHALYDKTKKIFLKAKSEEEIFAHLGLEYIQPSERNA

SEQ ID NO: 12 - protein, 381 aa, Elephantulus edwardii
DCPASHDSSPQKTESAAVQKISQYACQRRTTLNNHNHIFTDAFEILAENCEFRENEGSYVTYMRAASVLKSLPFSIISMKDTEGIPCLADKVKCVIEEIIEDGESSEVKAVLNDERYKSFKLFTSVFGVGLKTAEKWFRLGFRTLSGIMNDKTLKLTHMQKAGFLYYEDLVSCVTRAEAEAVGVLVKEAVWAFLPDAIVTMTGGFRRGKKVGHDVDFLITSPEATEEQEQQLLHKVITFWEKEGLLLYCDLYESTFEKLKMPSRKVDALDHFQKCFLILKLHRECVDDGTSSQLQGKTWKAIRVDLVVCPYECRAFALLGWTGSPQFERDLRRYATHERKMMLDNHALYDKTKRKFLSADSEEDIFAHLGLDYIEPWERNA

SEQ ID NO: 13 - protein, 387 aa, Python bivittatus
EKYQLPEDEDRSVTSDLDRDSISEYACQRRTTLKNYNQKFTDAFEILAENYEFNENKGFCTAFRRAASVLKCLPFTIVQVHDIEGVPWMGKQVKGIIEDIIEEGESSKVKAVLDNENYRSVKLFTSVFGVGLKTSDKWYRMGLRTLEEVKRDKNLKLTRMQKAGFLHYDDLTSCVSKAEADAASLIVQDVVWKIVPNAIVTIAGGFRRGKQTGHDVDFLITVPGSKQEEEELLHTVIDIWKKQELLLYYDLIESTFEDTKLPSRKVDALDHFQKCFAILKVHKEREDKGNSIRSKAFSEEEIKDWKAIRVDLVVVPFEQYAFALLGWTGSTQFERDLRRYATHEKKMMLDNHALYDKTKKIFLNAASEEEIFAHLGLDYLEPWERNA

SEQ ID NO: 14 - protein, 381 aa, Canis lupus familiaris
DYTASPNPELQKTLPVAVKKISQYACQRRTTLNNYNNVFTDAFEVLAENYEFRENEVFSLTFMRAASVLKSLPFTIISMKDTEGIPCLGDQVKCIIEEIIEDGESSEVKAVLNDERYQSFKLFTSVFGVGLKTSEKWFRMGFRTLSKIKSDKSLKFTPMQKAGFLYYEDLVSCVTRAEAEAVGVLVKEAVGAFLPDAFVTMTGGFRRGKKMGHDVDFLITSPGSTDEDEEQLLPKVINLWERKGLLLYCDLVESTFEKLKLPSRKVDALDHFQKCFLILKLHHQRVDGGKCSQQEGKTWKAIRVDLVMCPYERRAFALLGWTGSRQFERDLRRYASHERKMILDNHALYDKTKKIFLKAESEEEIFAHLGLDYIEPWERNA

SEQ ID NO: 15 - protein, 382 aa, Elephantulus edwardii
GDCPASHDSSPQKTESAAVQKISQYACQRRTTLNNHNHIFTDAFEILAENCEFRENEGSYVTYMRAASVLKSLPFSIISMKDTEGIPCLADKVKCVIEEIIEDGESSEVKAVLNDERYKSFKLFTSVFGVGLKTAEKWFRLGFRTLSGIMNDKTLKLTHMQKAGFLYYEDLVSCVTRAEAEAVGVLVKEAVWAFLPDAIVTMTGGFRRGKKVGHDVDFLITSPEATEEQEQQLLHKVITFWEKEGLLLYCDLYESTFEKLKMPSRKVDALDHFQKCFLILKLHRECVDDGTSSQLQGKTWKAIRVDLVVCPYECRAFALLGWTGSPQFERDLRRYATHERKMMLDNHALYDKTKRKFLSADSEEDIFAHLGLDYIEPWERNA

SEQ ID NO: 16 - protein, 379 aa, Ochotona princeps
EYSANPSPGPQATPAVYKISQYACQRRTTLNNHNHIFTDAFEILAENYEFKENEGCYVTYMRAASVLKSLPFTIVSMKDTEGIPCLEDKVKSIMEEIIEEGESSEVKAVLSDERYQCFKLFTSVFGVGLKTSEKWFRMGFRSLSNIRLDKSLKFTQMQKAGFRYYEDIVSCVTRAEAEAVDVLVNEAVRAFLPDAFITMTGGFRRGKKIGHDVDFLITSPELTEEDEQQLLHKVMNLWEKKGLLLYHDLVESTFEKLKQPSRKVDALDHFQKCFLIFKLYHERVGGDRCRQPEGKDWKAIRVDLVMCPYECHAFALLGWTGSRQFERDLRRYASHERKMILDNHALYDKTKRVFLQAENEEEIFAHLGLDYIEPWERNA

SEQ ID NO: 17 - protein, 384 aa, Erinaceus europaeus
DASFGSNPGSQNTPPLAIKKISQYACQRRTSLNNCNHIFTDALDILAENHEFRENEVSCVAFMRAASVLKSLPFTIISMKDTKGIPCLGDKAKCVIEEIIEDGESSEVKAILNDERYQSFKLFTSVFGVGLKTSEKWFRMGFRTLNKIMSDKTLKLTRMQKAGFLYYEDLVSCVAKAEADAVSVLVQEAVWAFLPDAMVTMTGGFRRGKKLGHDVDFLITSPGATEEEEQQLLPKVINFWERKGLLLYHDLVESTFEKLKLPSRKVDALDHFQKCFLILKLHLQHVNGVGNSKTGQQEGKNWKAIRVDLVMCPYERRAFALLGWTGSRQFERDLRRFATHERKMMLDNHALYDKTKRIFLKAESEEEIFAHLGLDYIDPWERNA

SEQ ID NO: 18 - protein, 381 aa, Tupaia chinensis
DHSTSPSPGPQKTPALAVQKISQYACQRRTTLNNCNRVFTDAFETLAENYEFRENEDSSVIFLRAASVLRSLPFTITSMRDTEGLPCLGDKVKCVIEEIIEDGESSEVNAVLNDERYKSFKLFTSVFGVGLKTSEKWFRMGFRTLSRVRSDKSLHLTRMQQAGFLYYEDLASCVTRAEAEAVGVLVKEAVGAFLPDALVTITGGFRRGKKTGHDVDFLITSPGSTEEKEEELLQKVLNLWEKKGLLLYYDLVESTFEKLKTPSRKVDALDHFPKCFLILKLHHQRVDGDKPSQQEGKSWKAIRVDLVMCPYERHAFALLGWTGSRQFERDLRRYATHERKMMLDNHALYDKTKRVFLKAESEEDIFAHLGLDYIEPWERNA

SEQ ID NO: 19 - protein, 394 aa, Ornithorhynchus anatinus
LTNSAPINCMTETPSLATKQVSQYACERRTTLNNCNQKFTDAFEILAKDFEFRENEGICLAFMRAISVLKCLPFTIVRMKDIEGVPWLGDQVKSIIEEIIEDGESSSVKAVLNDERYRSFQLFNSVFEVGLTDNGENGIARGFQTLNEVITDENISLTKTTLSTSLWNYLPGFLYYEDLVSCVAKEEADAVYLIVKEAVRAFLPEALVTLTGGFRRGKKIGHDVDFLISDPESGQDEQLLPNIIKLWEKQELLLYYDLVESTFEKTKIPSRKVDAMDHFQKCFLILKLHHQKVDSGRYKPPPESKNHEAKNWKAIRVDLVMCPFEQYAYALLGWTGSRQFERDLRRYATHEKKMMLDNHALYDKTKKIFLKAESEEDIFTHLGLDYIEPWERNA

SEQ ID NO: 20 - protein, 384 aa, Jaculus jaculus
SSELELLDVSWLIECMGAGKPVEMTGRHQLVKQTFCLPGFILQDAFDILAENCEFRENEASCVEFMRAASVLKSLPFPIISVKDTEGIPWLGGKVKCVIEEIIEDGESSEVKALLNDERYKSFKLFTSVFGVGLKTAERWFRMGFRTLSTVKLDKSLTFTRMQKAGFLHYEDLVSCVTRAEAEAVSVLVQQAVVAFLPDALVSMTGGFRRGKKIGHDVDFLITSPEATEEEEQQLLHKVTNFWEQKGLLLYCDHVESTFEKCKLPSRKVDALDHFQKCFLILKLYRERVDSVKSSQQEGKGWKAIRVDLVMCPYECRAFALLGWTGSRQFERDLRRYATHERKMRLDNHALYDKTKRVFLKAESEEEIFAHLGLEYIEPLERNA

SEQ ID NO: 21 - protein, 364 aa, Puma concolor
VVKKIPLYACQRRTTLNNFNHIFTDAFEVLAENYEFKENEISSATFMRAASVLKLPFTIISMKDTEGIPCLGDKVKCVIEEIIEDGESSEVKAVLNDERYQSFKLFTSVFGVGLKTSEKWFRMGFRTLSKIKSDKTLKFTQMQKAGFLYYEDLVSCVTRAEAEAVGVLVKEAVWAFLPDAFVTMTGGFRRGKKIGHDVDFLITIPGSTDEEEEQLLPKVINLWQRKELLLYYDLVESTFEKLKLPSRKVDALDHFQKCFLILKLHHQRVDSGKCSQQEGKTWKAIRVDLVMCPYERRAFALLGWTGSRQFERDLRRYATHERKMILDNHALYDKTKKIFLKAESEEEIFAHLGLDYIEPWERNA

SEQ ID NO: 22 - protein, 362 aa, Artificial Sequence
  N139 reptilian
YACQRRTTLNNYNKKYTDAFEILAENYEMRENAGACLAFRRAASVLKFLPFAIVRSNDIEGLPWMGEQVKGIIEDILEEGQSPKVEAVLNNEKYRSFKLFTSVFGVALKTSEKWFMMGLRNLEDVKLNQNLQLTRMQKAGLQHYEDLISYVSKAEADSTSLMVKDTVWKFSPSALVTLTGGFRRGKKMGHDVDFLITVPGSRPNEELLHLVIDCWKKQGLLLYYDLIESTFEKSKLPSQRVDALDHFQKCFAILKLHKERVNQGTSLPPVASTVEEIKDWKAIRVDLVVSPFEQHAFALLGWTGSRQFERDLRRYATHEKKMMLDNHALYDKTKKIFLSASSEEEIFAHLGLDYLEPWERNA

SEQ ID NO: 23 - protein, 364 aa, Elephantulus edwardii
VQKISQYACQRRTTLNNHNHIFTDAFEILAENCEFRENEGSYVTYMRAASVLKSLPFSIISMKDTEGIPCLADKVKCVIEEIIEDGESSEVKAVLNDERYKSFKLFTSVFGVGLKTAEKWFRLGFRTLSGIMNDKTLKLTHMQKAGFLYYEDLVSCVTRAEAEAVGVLVKEAVWAFLPDAIVTMTGGFRRGKKVGHDVDFLITSPEATEEQEQQLLHKVITFWEKEGLLLYCDLYESTFEKLKMPSRKVDALDHFQKCFLILKLHRECVDDGTSSQLQGKTWKAIRVDLVVCPYECRAFALLGWTGSPQFERDLRRYATHERKMMLDNHALYDKTKRKFLSADSEEDIFAHLGLDYIEPWERNA

SEQ ID NO: 24 - protein, 382 aa, Serinus canaria
SPPLNTPELEMPSFIATKVSQYSCQRKTTLNNYNKKFTDAFEVMAENYEFKENEIFCLEFLRAASLLKSLPFSVTRMKDIQGLPCMGDQVRDVIEIIEEGESSRVKEVLNDERYKAFKQFTSVFGVGVKTSEKWYRMGLRTVGEVKADKTLKLSKMQKAGFLYYEDLVSCVSKAEADAVSLIVKNTVCTFLPDALVTITGGFRRGKNIGHDIDFLITNPGPREDDELLHKVIDLWKKQGLLLYCDIIESTFVKEQLPSRKIDAMDHFQKCFAILKLYQPRVDNSTCNTSKKLEMAEVKDWKAIRVDLVITPFEQYSYALLGWTGSRQFGRDLRRYAAHERRMILDNHGLYDRTKRIFLKAGSEEEIFAHLGLDYVEPWERNA

SEQ ID NO: 25 - protein, 370 aa, Neopelma chrysocephalum
FIARKVSQYSCQRKTTLNNYNKKFTDAFEIMAENYEFKENEIFCLEFLRAASLLKYLPFPVTRMKDIQGLPCIGDQVRDVIEGIIEEGESSRVKEVLNDERYKAFKQFTSVFGVGVKTSEKWYRMGLRTVEELKADKTLKLSKMQKAGFLYYEDLVSCVSKAEADAVTLIVKNTVSTFLPDALVTITGGFRRGKKMGHDIDFLITNPGPREDDELLHKVVDLWKKQGLLLYCDIIESTFVEEQLPSRKVDAMDNFQKCFTILKLYQPGVDNSSYNMSKKSDMAEVKDWKAIRVDLVITPFEQYAYALLGWTGSREFGRDLRRYASHERKMILDNHGLYDRRKRIFLKAGSEEEIFAHLGLDYVEPWERNA

SEQ ID NO: 26 - protein, 383 aa, Alligator sinensis
SPSPVPGSQNVPAPSVDKVSQYACQRRTTLNNYNKKFTDAFEILAENCEFRENRLGCLEFLRAASVLKFLPFPIVKMKNIEGLPCMGDKVKCVIEEILEEGESCQAKEILNDERYKSFKLFTSVFGVGLKTTEKWYRMGFRTLEEVKAEKTLKLSRMQIAGFLHYEDIISYVSKAEADAVSLLIKDTVCMFLPDALVTITGGFRRGKKTGHDVDFLITNPGPEEEKELLHKVVDLWEKQGLLLYYDVIESTFEKEKRPSRKVDALDHFQKCFAILKLHQQRRGNSNSNISKESDKAEVKDWKAIRVDLVISPFEQYAYALLGWTGSRQFERDLRRYASRERKMMLDNHALYDKTKRTFLKAESEEEIFAHLGLDYIEPWERNA

SEQ ID NO: 27 - protein, 380 aa, Xenopus laevis
SPSPVPGSQNVPAPSEVKVSQYACQRCTTLQDTNRIFTDAFDILAEHFEFCENKGRTVAFLRASSLIKSLPFPITAMKELEGLPWLGDQMKGIIEEILEEGKSYKVLEVMNEERYKSFKQFTSVFGVGLKTSDKWFRMGFRTLEEIKNEKELKLTKMQKCGLLYYEDITSYVSRAEAETTEQLIKSIVWKFVPDAIVTLTGGFRRGKKKGHDVDILITCARKGKEKNILHNTMSVLKNRGLLLFYNIIESTFDETKLPSRHVDALDHFQKCFTILKLPKRQMDIGNIIDPHECERKNWKAVRLDLVITPYEQYPYALLGWTGSRQFERDLRRYATHEKRMMLDNHGLYDKTKNNFLKANNEEDIFKQLGLDYLEPWERNA

SEQ ID NO: 28 - protein, 383 aa, Notechis scutatus
SPSPVPGSQNVPAPSVDSISPYACQRRTTLKNYNQKFTDAFEILVENYEFNENKEFCLAFGRAASLLKCLPFTVVRVNDIEGLPWMGKQVREIIEDILEEGESSKVKAVLNDENYRSIKLFTSVFGVGLRTSEKWYRMGLRTLEEVKCNKNLTLTRMQKAGFFYYDDLISSVSKAEADAATQIVQDTVWKILPNAVVTLTGGFRRGKQTGHDVDFLITVPGSRQEEELLHPVIDIWKKQELLLYYDLIESTFENTKLPSRKVDALDHFQKCFAILKVHKERVNKGSAVQSNVFAEEGTKDWKAIRVDLVVTPFQHYAFALLGWTGSRQFERDLRRYATQEKKMMLDNHALYDKTKKIFLSAANEEEIFSHLGLDYLEPWERNA

SEQ ID NO: 29 - protein, 367 aa, Salmo truttal
TAAANVSQYACLRRTTTENHNKIFTDVLEELAENSEFNESKGPCLAFRRAASVLKSLPSAVHCLGAIQGLPCLGEHTKAVMEEILIFGRSFKVEEVQSDERYQALKLFTSVFGVGPKTAEKWYRRGLRSLKEILAEPNIQLNRMQRAGFLYYRDISKAVSKAEAKALSSIIEETAHWIAPDSILALTGGFRRGKEYGHDVDFLLTMPEMGKEEGLLLRVIDRLRDQGILLYCEHQDSTFDMSKLPSHRFEAMDHFEKCFLILRLEEGQVEGDGGLQKDPGESRGWRAVRVDLVAPPVDRYAFVLLGWTGSRQFERDLRRFASKERGMCLDNHALYDKTKKLFLPATSEEDIFAHLGLEYVEPWQRNA

SEQ ID NO: 30 - protein, 377 aa, Electrophorus electricus
SPSPVPGSQNVPAPSLPTVSQYACQRRTTLDNHNKVFTDALEVLIENYEFSDNKGACVGFRRAASVLKSLPKPLRCLKDMEGLPCLGDDTKAIIEEIYECGSSSRVENILSDEKYQTLKLFTSVFGVGPKTGEKWYRRGLRALEQVHSEPSIQLNKMQAAGFLYYEDISKPVSRAEAKAVGCIIEEVASCFSSSVTITLTGGFRRGKEFGHDVDFLLSIPEPGKEDGLLPAVIDRLRKQGILLYSDLQESTLQQWKRPSRCFDSMDHFQKCFLIVKLWTRLVEGHREDPSSQRDWKAVRVDLVVPPVDCYAFALLGWSGSTQFERDLRRFARLERRMLLDNHALYDKTTNTFLQAKTEEDIFAHLGLDYIEPWQRNA

SEQ ID NO: 31 - protein, 378 aa, Anabas testudineus
SPSPVPGSQNVPAPSVATVSQYACQRRTTTENNNKILTDAFEVLAESYELNQLEGPCLAFRRAASVLKSLPWAVQCLGATQGLPCLGEHTKALIEEILQYGHSFEVEKILSDERYQTLKLFTSVFGVGPKTAEKWYRRGLRSFSDILAEPSIQLNRMQQSGFLHYGDISRAVSKAEARALGNIIDEAVHAITPDGILALTGGFRRGKEFGHDVDFIVTTPEQGKEETLLPNIIDRLKEQGILLYSDYQTSTFDISKLPSHKFEAMDHFAKCFLILRLEGSLVDRGLNSTEGDSRGWRAVRVDLVSPPMERYAYALLGWTGSRQFERDLRRFARLEQHMLLDNHALYDKTKKEFLAATTERDIFAHLGLEYIEPWQRNA

SEQ ID NO: 32 - protein, 378 aa, Poecilia reticulata
SPSPVPGSQNVPAPSVDKVSQYACQRRTTVENNNRIFTDAFEVLAENYEFNEIEGRCLAFRRAASVLKSLPWAVRSVGATLDLPCLGEHTTAVMKEILQYGRSFEVEKILSDERCQTLKLFTSVFGVGPKTAEKWYRRGLRSFSDVLAQPGIHLNRMQQSGFLHYGDISRAVSKAEARAVGNIIDEAVHVITPNAILALTGGFRRGKDFGHDVDFIVTTTELGKEKNLLISVIESLKKQGLLLFSDYQASTFDISKLPSHRFEAMDHFAKCFLILRLEGSRVEGGLQRAQADGRGWRAVRVDLVSPPADRFAFTMLGWTGSRMFERDLRRFARLERQMLLDNHALYDKTKKEFLTAATEKDIFDHLGLEYIEPWQRNA

SEQ ID NO: 33 - protein, 366 aa, Rattus norvegicus
PLMQKISQYACQRRTTLNNHNQLFTDAFDILAENYEFRENEVSCLPFMRAASVLKSLSFPIVSMKDIEGIPCLGDKVKCVIEGIIEDGESSEVKAVLNDERYKSFKLFTSVFGVGLKTAEKWFRMGFRTLSKIKSDKSLRFTHMQKAGFLYYEDLVSCVNRAEAEAVSMLVKEAVVAFLPDALVTMTGGFRRGKMTGHDVDFLITSPEATEEEEQQLLHKVTNFWRQQGLLLYCDIIESTFEKFKLPSRKVDALDHFQKCFLILKLHRGLVRSEESGQQEGKDWKAIRVDLVMCPYERRAFALLGWTGSRQFERDLRRYATHERKMMLDNHALYDKTKRVFLEAESEEEIFAHLGLDYIEPWERNA

SEQ ID NO: 34 - protein, 379 aa, Rattus norvegicus
SLSPVPGSQTVPPPLMQKISQYACQRRTTLNNHNQLFTDAFDILAENYEFRENEVSCLPFMRAASVLKSLSFPIVSMKDIEGIPCLGDKVKCVIEGIIEDGESSEVKAVLNDERYKSFKLFTSVFGVGLKTAEKWFRMGFRTLSKIKSDKSLRFTHMQKAGFLYYEDLVSCVNRAEAEAVSMLVKEAVVAFLPDALVTMTGGFRRGKMTGHDVDFLITSPEATEEEEQQLLHKVTNFWRQQGLLLYCDIIESTFEKFKLPSRKVDALDHFQKCFLILKLHRGLVRSEESGQQEGKDWKAIRVDLVMCPYERRAFALLGWTGSRQFERDLRRYATHERKMMLDNHALYDKTKRVFLEAESEEEIFAHLGLDYIEPWERNA

SEQ ID NO: 35 - protein, 379 aa, Piliocolobus tephrosceles
SDSTNPGLPKTPPTAIQKISQYACQRRTTLNNCNQIFTDAFDILAENCEFRENEDSCVTFMRAASVLKSLPFTIISMKDTEGIPCLGSKVKCIIEEIIEDGESSEVKAVLNDERYQSFKLFTSVFGVGLKTSEKWFRMGFRTLSKVRSDESLKFTRMQRAGFLYYEDLVSCVTRAEAEAVSALVKEAVWAFLPDAFVTMTGGFRRGKKMGHDVDFLITSPGSTEDEEQQLLQKVMNLWEKKGLLLYYDLVESTFEKLRLPSRKVDALDHFQKCFLIFKLPLQRVDSDQSSWQGGKTWKAIRVDLVMCPYERRAFALLGWTGSRQFERDLRRYATHERKMILDNHALYDKTKRIFLKAESEEEIFAHLGLDYIEPWERNA

SEQ ID NO: 36 - protein, 379 aa, Sus scrofa
SASPSPGSQNTLPPAVKKISQYACQRRTTLNNCNHIFTDAFEVLAENYEFRENETFCLAFMRAASVLKSLPFTIISMKDTEGIPCLGDKVKCVIEEIIEDGESSEVKAVLNDERYQSFKLFTSVFGVGLKTSERWFRMGFRSLSKIRSDKTLKFTRMQKAGFLYYEDLVSCVTRAEAEAVGVLVKEAVQAFLPDAFVTMTGGFRRGKKMGHDVDFLITSPGSTDDEEQQLLPKVVNLWEREGLLLYCDLVESTLEKSKLPSRNVDALDHFQKCFLILKLHHQRVDSGMSSQQEGKTWKAIRVDLVMCPYELRAFALLGWTGSRQFERDLRRYATHERKMILDNHALYDKTKRIFLKAESEEEIFAHLGLDYLEPWERNA

SEQ ID NO: 37 - protein, 379 aa, Panthera tigris altaica
YSASPNPELQKTPPLVKKIPLYACQRRTTLNNFNHVFTDAFEVLAENYEFKENEVSSATFMRAASVLKSLPFTIISMKDTEGIPCLGDKVKCVIEEIIEDGESSEVKAVLNDERYQSFKLFTSVFGVGLKTSEKWFRMGFRTLSKIKSDKTLKFTQMQKAGFLYYEDLVSCVTRAEAEAVGVLVKEAVWAFLPDAFVTMTGGFRRGKKIGHDVDFLITSPGSTDEEEEELLPKVINLWQRKELLLYYDLVESTFEKLKLPSRKVDALDHFQKCFLILKLHHQRVDSGKCSQQEGKTWKAIRVDLVMCPYERRAFALLGWTGSRQFERDLRRYATHERKMILDNHALYDKTKKIFLKAESEEEIFAHLGLDYIEPWERNA

SEQ ID NO: 38 - protein, 365 aa, Bubalus bubalis
LAVKKISQYACQRKTTLNNYNHIFTDAFEILAENSEFKENEVSYVTFMRAASVLKSLPFTIISMKDTQGIPCLGDKVKCVIEEIIEDGESSEVKAVLNDERYQSFKLFTSVFGVGLKTSEKWFRMGFRSLSKITSDKTLKFTKMQKAGFLYYEDLVSCVTRAEAEAVGVLVKEAVWAFLPDAFITMTGGFRRGKKIGHDVDFLITSPGSAEDEEQLLPKVINLWEKKGLLLYYDLVESTFEKFKLPSRQVDTLDHFQKCFLILKLHHQRVDSGRSNQQEGKTWKAIRVDLVMCPYENRAFALLGWTGSRQFERDIRRYATHERKMMLDNHALYDKTKRVFLKAESEEEIFAHLGLDYIEPWERNA

SEQ ID NO: 39 - protein, 379 aa, Marmota flaviventris
PDHSNSDPQRIPPPAVQTISQYACQRRTTLNNCNRVFTDAFDVLAENYEFRENESCSVVFMRAASVLKSLPFTIISMRDLEGIPCLEGKAKSIIEEIIEDGESSEVKAVLNDERYKSFKLFTSVFGVGLKTSEKWFRMGFRTLSKIRSDKSLKFTHMQKAGFLYYEDLVSCVTRAEAEAVSVLVKEAVWAFLPDAFITMTGGFRRGKNIGHDVDFLITSAEATEEEEQQLLHKVTNLWEKKGLLLYCDLVESTFEKLKTPSRKVDALDHFQKCFLILKLHHQRVDSDKASQQGGKNWKAIRVDLVMCPYERRAFALLGWTGSRQFERDLRRYATHERKMILDNHALYDKTKRIFLKAESEEEIFAHLGLDYIEPWERNA

SEQ ID NO: 40 - protein, 362 aa, Mus musculus
KISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKRVFLEAESEEEIFAHLGLDYIEPWERNA

SEQ ID NO: 41 - protein, 361 aa, Bos taurus
KISQYACQRKTTLNNYNHIFTDAFEILAENSEFKENEVSYVTFMRAASVLKSLPFTIISMKDTEGIPCLGDKVKCIIEEIIEDGESSEVKAVLNDERYQSFKLFTSVFGVGLKTSEKWFRMGFRSLSKIMSDKTLKFTKMQKAGFLYYEDLVSCVTRAEAEAVGVLVKEAVWAFLPDAFVTMTGGFRRGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSRQFERDIRRYATHERKMMLDNHALYDKTKRVFLKAESEEEIFAHLGLDYIEPWERNA

SEQ ID NO: 42 - protein, 361 aa, Homo sapiens
KISQYACQRRTTLNNCNQIFTDAFDILAENCEFRENEDSCVTFMRAASVLKSLPFTIISMKDTEGIPCLGSKVKGIIEEIIEDGESSEVKAVLNDERYQSFKLFTSVFGVGLKTSEKWFRMGFRTLSKVRSDKSLKFTRMQKAGFLYYEDLVSCVTRAEAEAVSVLVKEAVWAFLPDAFVTMTGGFRRGKKMGHDVDFLITSPGSTEDEEQLLQKVMNLWEKKGLLLYYDLVESTFEKLRLPSRKVDALDHFQKCFLIFKLPRQRVDSDQSSWQEGKTWKAIRVDLVLCPYERRAFALLGWTGSRQFERDLRRYATHERKMILDNHALYDKTKRIFLKAESEEEIFAHLGLDYIEPWERNA

SEQ ID NO: 43 - protein, 358 aa, Gallus gallus
KVSQYSCQRKTTLNNCNKKFTDAFEIMAENYEFKENEIFCLEFLRAASVLKSLPFPVTRMKDIQGLPCMGDRVRDVIEEIIEEGESSRAKDVLNDERYKSFKEFTSVFGVGVKTSEKWFRMGLRTVEEVKADKTLKLSKMQRAGFLYYEDLVSCVSKAEADAVSSIVKNTVCTFLPDALVTITGGFRRGKKIGHDIDFLITSPGQREDDELLHKGLLLYCDIIESTFVKEQIPSRHVDAMDHFQKCFAILKLYQPRVDNSSYNMSKKCDMAEVKDWKAIRVDLVITPFEQYAYALLGWTGSRQFGRDLRRYATHERKMMLDNHALYDKRKRVFLKAGSEEEIFAHLGLDYVEPWERNA

SEQ ID NO: 44 - protein, 368 aa, Artificial Sequence
  Monodelphis domestica truncated  M63 C173 R207 R331 E334
TISQYACQRRTTINNHNQRFTDAFEILAKNYEFKENDDTCLTFMRAISVLKCLPFEVVSLKDTEGLPWIGDEVKGIMEEIIEDGESLEVQAVLNDERYQSFKLFTSVFGVGLKTADKWYRMGFRTLNKIRSDKTLKLTKMQKAGLCYYEDLIDCVSKAEADAVSLLVQDAVWTFLPDALVTITGGFRRGKEFGHDVDFLITSPGAEKEQEDQLLQKVTNLWKKQGLLLYCDLIESTFEDLKLPSRKIDALDHFQKCFLILKLYHHKEDKRKWEMPTGSNESEAKSWKAIRVDLVVCPYDRYAFALLGWSGSRQFERDLRRYATHEKKMMLDNHALYDKTKKIFLKAKSEEEIFAHLGLEYIQPSERNA

SEQ ID NO: 45 - protein, 362 aa, Elephantulus edwardii
KISQYACQRRTTLNNHNHIFTDAFEILAENCEFRENEGSYVTYMRAASVLKSLPFSIISMKDTEGIPCLADKVKCVIEEIIEDGESSEVKAVLNDERYKSFKLFTSVFGVGLKTAEKWFRLGFRTLSGIMNDKTLKLTHMQKAGFLYYEDLVSCVTRAEAEAVGVLVKEAVWAFLPDAIVTMTGGFRRGKKVGHDVDFLITSPEATEEQEQQLLHKVITFWEKEGLLLYCDLYESTFEKLKMPSRKVDALDHFQKCFLILKLHRECVDDGTSSQLQGKTWKAIRVDLVVCPYECRAFALLGWTGSPQFERDLRRYATHERKMMLDNHALYDKTKRKFLSADSEEDIFAHLGLDYIEPWERNA

SEQ ID NO: 46 - protein, 362 aa, Canis lupus familiaris
KISQYACQRRTTLNNYNNVFTDAFEVLAENYEFRENEVFSLTFMRAASVLKSLPFTIISMKDTEGIPCLGDQVKCIIEEIIEDGESSEVKAVLNDERYQSFKLFTSVFGVGLKTSEKWFRMGFRTLSKIKSDKSLKFTPMQKAGFLYYEDLVSCVTRAEAEAVGVLVKEAVGAFLPDAFVTMTGGFRRGKKMGHDVDFLITSPGSTDEDEEQLLPKVINLWERKGLLLYCDLVESTFEKLKLPSRKVDALDHFQKCFLILKLHHQRVDGGKCSQQEGKTWKAIRVDLVMCPYERRAFALLGWTGSRQFERDLRRYASHERKMILDNHALYDKTKKIFLKAESEEEIFAHLGLDYIEPWERNA

SEQ ID NO: 47 - protein, 362 aa, Elephantulus edwardii; identical to SEQ ID NO: 45

SEQ ID NO: 48 - protein, 362 aa, Ochotona princeps
KISQYACQRRTTLNNHNHIFTDAFEILAENYEFKENEGCYVTYMRAASVLKSLPFTIVSMKDTEGIPCLEDKVKSIMEEIIEEGESSEVKAVLSDERYQCFKLFTSVFGVGLKTSEKWFRMGFRSLSNIRLDKSLKFTQMQKAGFRYYEDIVSCVTRAEAEAVDVLVNEAVRAFLPDAFITMTGGFRRGKKIGHDVDFLITSPELTEEDEQQLLHKVMNLWEKKGLLLYHDLVESTFEKLKQPSRKVDALDHFQKCFLIFKLYHERVGGDRCRQPEGKDWKAIRVDLVMCPYECHAFALLGWTGSRQFERDLRRYASHERKMILDNHALYDKTKRVFLQAENEEEIFAHLGLDYIEPWERNA

SEQ ID NO: 49 - protein, 365 aa, Erinaceus europaeus
KISQYACQRRTSLNNCNHIFTDALDILAENHEFRENEVSCVAFMRAASVLKSLPFTIISMKDTKGIPCLGDKAKCVIEEIIEDGESSEVKAILNDERYQSFKLFTSVFGVGLKTSEKWFRMGFRTLNKIMSDKTLKLTRMQKAGFLYYEDLVSCVAKAEADAVSVLVQEAVWAFLPDAMVTMTGGFRRGKKLGHDVDFLITSPGATEEEEQQLLPKVINFWERKGLLLYHDLVESTFEKLKLPSRKVDALDHFQKCFLILKLHLQHVNGVGNSKTGQQEGKNWKAIRVDLVMCPYERRAFALLGWTGSRQFERDLRRFATHERKMMLDNHALYDKTKRIFLKAESEEEIFAHLGLDYIDPWERNA

SEQ ID NO: 50 - protein, 362 aa, Tupaia chinensis
KISQYACQRRTTLNNCNRVFTDAFETLAENYEFRENEDSSVIFLRAASVLRSLPFTITSMRDTEGLPCLGDKVKCVIEEIIEDGESSEVNAVLNDERYKSFKLFTSVFGVGLKTSEKWFRMGFRTLSRVRSDKSLHLTRMQQAGFLYYEDLASCVTRAEAEAVGVLVKEAVGAFLPDALVTITGGFRRGKKTGHDVDFLITSPGSTEEKEEELLQKVLNLWEKKGLLLYYDLVESTFEKLKTPSRKVDALDHFPKCFLILKLHHQRVDGDKPSQQEGKSWKAIRVDLVMCPYERHAFALLGWTGSRQFERDLRRYATHERKMMLDNHALYDKTKRVFLKAESEEDIFAHLGLDYIEPWERNA

SEQ ID NO: 51 - protein, 375 aa, Ornithorhynchus anatinus
QVSQYACERRTTLNNCNQKFTDAFEILAKDFEFRENEGICLAFMRAISVLKCLPFTIVRMKDIEGVPWLGDQVKSIIEEIIEDGESSSVKAVLNDERYRSFQLFNSVFEVGLTDNGENGIARGFQTLNEVITDENISLTKTTLSTSLWNYLPGFLYYEDLVSCVAKEEADAVYLIVKEAVRAFLPEALVTLTGGFRRGKKIGHDVDFLISDPESGQDEQLLPNIIKLWEKQELLLYYDLVESTFEKTKIPSRKVDAMDHFQKCFLILKLHHQKVDSGRYKPPPESKNHEAKNWKAIRVDLVMCPFEQYAYALLGWTGSRQFERDLRRYATHEKKMMLDNHALYDKTKKIFLKAESEEDIFTHLGLDYIEPWERNA

SEQ ID NO: 52 - protein, 365 aa, Serinus canaria
KVSQYSCQRKTTLNNYNKKFTDAFEVMAENYEFKENEIFCLEFLRAASLLKSLPFSVTRMKDIQGLPCMGDQVRDVIEIIEEGESSRVKEVLNDERYKAFKQFTSVFGVGVKTSEKWYRMGLRTVGEVKADKTLKLSKMQKAGFLYYEDLVSCVSKAEADAVSLIVKNTVCTFLPDALVTITGGFRRGKNIGHDIDFLITNPGPREDDELLHKVIDLWKKQGLLLYCDIIESTFVKEQLPSRKIDAMDHFQKCFAILKLYQPRVDNSTCNTSKKLEMAEVKDWKAIRVDLVITPFEQYSYALLGWTGSRQFGRDLRRYAAHERRMILDNHGLYDRTKRIFLKAGSEEEIFAHLGLDYVEPWERNA

SEQ ID NO: 53 - protein, 366 aa, Neopelma chrysocephalum
KVSQYSCQRKTTLNNYNKKFTDAFEIMAENYEFKENEIFCLEFLRAASLLKYLPFPVTRMKDIQGLPCIGDQVRDVIEGIIEEGESSRVKEVLNDERYKAFKQFTSVFGVGVKTSEKWYRMGLRTVEELKADKTLKLSKMQKAGFLYYEDLVSCVSKAEADAVTLIVKNTVSTFLPDALVTITGGFRRGKKMGHDIDFLITNPGPREDDELLHKVVDLWKKQGLLLYCDIIESTFVEEQLPSRKVDAMDNFQKCFTILKLYQPGVDNSSYNMSKKSDMAEVKDWKAIRVDLVITPFEQYAYALLGWTGSREFGRDLRRYASHERKMILDNHGLYDRRKRIFLKAGSEEEIFAHLGLDYVEPWERNA

SEQ ID NO: 54 - protein, 366 aa, Alligator sinensis
KVSQYACQRRTTLNNYNKKFTDAFEILAENCEFRENRLGCLEFLRAASVLKFLPFPIVKMKNIEGLPCMGDKVKCVIEEILEEGESCQAKEILNDERYKSFKLFTSVFGVGLKTTEKWYRMGFRTLEEVKAEKTLKLSRMQIAGFLHYEDIISYVSKAEADAVSLLIKDTVCMFLPDALVTITGGFRRGKKTGHDVDFLITNPGPEEEKELLHKVVDLWEKQGLLLYYDVIESTFEKEKRPSRKVDALDHFQKCFAILKLHQQRRGNSNSNISKESDKAEVKDWKAIRVDLVISPFEQYAYALLGWTGSRQFERDLRRYASRERKMMLDNHALYDKTKRTFLKAESEEEIFAHLGLDYIEPWERNA

SEQ ID NO: 55 - protein, 366 aa, Alligator sinensis
KISQYACQRRTTLNNYNKKFTDAFEILAENYEFNENKGFCLAFRRAASVLKFLPFTIVRMNDIQGLPWMGDQVKRVIEEILEEGESSKVKEVLNDEKYKAFKLFTSVFGVGRKTSEKWYRMGLRTLEEVKADKTLKLTKMQKAGFLYYEDLISRVSKAEADAVSLIVKDTVWKFLPDALVTLTGGFRLGKKMGHDVDFLITTPGPEEEEELLHKVIDLWKKQGLLLYYDIIESTFEKTKLPSRTVDALDHFQKCFAILKLHKQRVDNGNSNQSKEFDKEETKDWKAIRVDLVITPFEQYAYALLGWTGSAFFNRDLRRYATHEKKMMLDNHALYDKTKRIFLKAASEEEIFAHLGLDYLEPWERNA

SEQ ID NO: 56 - protein, 363 aa, Xenopus laevis
KVSQYACQRCTTLQDTNRIFTDAFDILAEHFEFCENKGRTVAFLRASSLIKSLPFPITAMKELEGLPWLGDQMKGIIEEILEEGKSYKVLEVMNEERYKSFKQFTSVFGVGLKTSDKWFRMGFRTLEEIKNEKELKLTKMQKCGLLYYEDITSYVSRAEAETTEQLIKSIVWKFVPDAIVTLTGGFRRGKKKGHDVDILITCARKGKEKNILHNTMSVLKNRGLLLFYNIIESTFDETKLPSRHVDALDHFQKCFTILKLPKRQMDIGNIIDPHECERKNWKAVRLDLVITPYEQYPYALLGWTGSRQFERDLRRYATHEKRMMLDNHGLYDKTKNNFLKANNEEDIFKQLGLDYLEPWERNA

SEQ ID NO: 57 - protein, 363 aa, Xenopus sp.
KVSQYACQRRTTLQDTNRIFTDAFDILAENYEFNENKGPCVAFRRASSVLKSLPFPIVAMKDLEGLPWLGDQMKRIIEEILEEGKSSKVVEVMNDERYKAFKLFTSVFGVGRKTSEKWFRMGFRTLEEIKSDKELKLTKMQKAGLLYYEDITSAVSKAEAEAVEQLIKSIVWKFVPDAIVTLTGGFRLGKKMGHDVDILITTPRKGEKEEILHKTINVLKKRGLLLFYNIIESTFDETKLPSRHVDALDHFQKCFTILKLQKQQLDNGNSNEPFETEKKNWKAIRVDLVITPYEQYAYALLGWTGSPQFNRDLRRYATHEKRMMLDNHALYDKTKNIFLKANSEEDIFTHLGLDYLEPWERNA

SEQ ID NO: 58 - protein, 367 aa, Notechis sp.
SISQYACQRRTTLNNYNKKFTDAFEILAENYEFNENKGFCLAFRRAASVLKFLPFTIVRVNDIEGLPWMGEQVKRIIEDILEEGESSKVKAVLNNENYRAFKLFTSVFGVGRKTSEKWYRMGLRTLEEVKADKNLKLTRMQKAGFLHYEDLISRVSKAEADAASLIVKDTVWKFLPNALVTLTGGFRLGKKMGHDVDFLITVPGSRQEEEELLHTVIDLWKKQGLLLYYDLIESTFEKTKLPSRTVDALDHFQKCFAILKLHKERVDKGNSIQSKAFAEEETKDWKAIRVDLVVTPFEQYAFALLGWTGSPFFNRDLRRYATHEKKMMLDNHALYDKTKKIFLKAASEEEIFAHLGLDYLEPWERNA

SEQ ID NO: 59 - protein, 363 aa, Salmo trutta
NVSQYACLRRTTTENHNKIFTDVLEELAENSEFNESKGPCLAFRRAASVLKSLPSAVHCLGAIQGLPCLGEHTKAVMEEILIFGRSFKVEEVQSDERYQALKLFTSVFGVGPKTAEKWYRRGLRSLKEILAEPNIQLNRMQRAGFLYYRDISKAVSKAEAKALSSIIEETAHWIAPDSILALTGGFRRGKEYGHDVDFLLTMPEMGKEEGLLLRVIDRLRDQGILLYCEHQDSTFDMSKLPSHRFEAMDHFEKCFLILRLEEGQVEGDGGLQKDPGESRGWRAVRVDLVAPPVDRYAFVLLGWTGSRQFERDLRRFASKERGMCLDNHALYDKTKKLFLPATSEEDIFAHLGLEYVEPWQRNA

SEQ ID NO: 60 - protein, 363 aa, Artificial Sequence
  F15 trunc new
TVSQYACQRRTTMENHNKIFTDAFEVLAENSEFNESKESRDAFRRAASVLKSLPSAVRSLGDTEGLPCLGEHTKRVIEEILEYGRSSKVEDILSDERYQTMKLFTSVFGVGPKTAEKWYRRGLRSLEEVRAEPSIHLNRMQRAGFLYYRDISKRVSKAEAKALGNIIEETVHAIAPDAIVTLTGGFRLGKEFGHDVDFILTTPEMGKEEGLLPRVIDRLRNQGILLYCDYQESTFDMSKLPSRTFEAMDHFQKCFLIIKLKEGLVEGEGGLQSDPGDRRGWRAVRVDLVAPPVDRYAFALLGWTGSPQFNRDLRRFARLERRMLLDNHALYDKTKKIFLPAKTEEDIFAHLGLEYIEPWQRNA

SEQ ID NO: 61 - protein, 360 aa, Electrophorus electricus
TVSQYACQRRTTLDNHNKVFTDALEVLIENYEFSDNKGACVGFRRAASVLKSLPKPLRCLKDMEGLPCLGDDTKAIIEEIYECGSSSRVENILSDEKYQTLKLFTSVFGVGPKTGEKWYRRGLRALEQVHSEPSIQLNKMQAAGFLYYEDISKPVSRAEAKAVGCIIEEVASCFSSSVTITLTGGFRRGKEFGHDVDFLLSIPEPGKEDGLLPAVIDRLRKQGILLYSDLQESTLQQWKRPSRCFDSMDHFQKCFLIVKLWTRLVEGHREDPSSQRDWKAVRVDLVVPPVDCYAFALLGWSGSTQFERDLRRFARLERRMLLDNHALYDKTTNTFLQAKTEEDIFAHLGLDYIEPWQRNA

SEQ ID NO: 62 - protein, 360 aa, Artificial Sequence
  F24 trunc new
TVSQYACQRRTTLDNHNKIFTDALEVLAENYEFSDSKESRDAFRRAASVLKSLPTPLRSLQDTEGLPCLGEETKRVIEEIFEEGSSSRVEDILSDERYQTLKLFTSVFGVGPKTAEKWYRRGLRSLEQVRSDPSIHLNRMQTAGFLYYEDISKPVSKAEAKALGNIIEETASAFSPSVTITLTGGFRLGKEFGHDVDFLLTVPEPGKEDGLLPAVIDRLRSQGILLYSDFQESTFDLSKLPSRRFEAMDHFQKCFLIVKLKAGLVEGEGADPGDRRDWRAVRVDLVAPPVDRYAFALLGWSGSAFFNRDLRRFARLERGMLLDNHALYDKTTNTFLQAKTEEDIFAHLGLDYIEPWQRNA

SEQ ID NO: 63 - protein, 361 aa, Anabas testudineus
TVSQYACQRRTTTENNNKILTDAFEVLAESYELNQLEGPCLAFRRAASVLKSLPWAVQCLGATQGLPCLGEHTKALIEEILQYGHSFEVEKILSDERYQTLKLFTSVFGVGPKTAEKWYRRGLRSFSDILAEPSIQLNRMQQSGFLHYGDISRAVSKAEARALGNIIDEAVHAITPDGILALTGGFRRGKEFGHDVDFIVTTPEQGKEETLLPNIIDRLKEQGILLYSDYQTSTFDISKLPSHKFEAMDHFAKCFLILRLEGSLVDRGLNSTEGDSRGWRAVRVDLVSPPMERYAYALLGWTGSRQFERDLRRFARLEQHMLLDNHALYDKTKKEFLAATTERDIFAHLGLEYIEPWQRNA

SEQ ID NO: 64 - protein, 361 aa, Anabas testudineus
TVSQYACQRRTTTENNNKIFTDAFEVLAESYEFNEMEESRDAFRRAASVLKSLPWAVQSLGATQDLPCLGEHTKRVMEEILQYGRSFEVEKILSDERYQTLKLFTSVFGVGPKTAEKWYRRGLRSFEDIRAEPSIHLNRMQQSGFLHYGDISRAVSKAEARALGNIIDEAVHAITPDAILALTGGFRLGKEFGHDVDFIVTTPELGKEESLLPNIIDRLKKQGILLYSDYQASTFDMSKLPSHRFEAMDHFAKCFLILRLEGSQVEGGLNSAEGDSRGWRAVRVDLVSPPMDRYAFALLGWTGSAFFNRDLRRFARMERRMLLDNHALYDKTKKEFLAATTEKDIFAHLGLEYIEPWQRNA

SEQ ID NO: 65 - protein, 361 aa, Poecilia reticulata
KVSQYACQRRTTVENNNRIFTDAFEVLAENYEFNEIEGRCLAFRRAASVLKSLPWAVRSVGATLDLPCLGEHTTAVMKEILQYGRSFEVEKILSDERCQTLKLFTSVFGVGPKTAEKWYRRGLRSFSDVLAQPGIHLNRMQQSGFLHYGDISRAVSKAEARAVGNIIDEAVHVITPNAILALTGGFRRGKDFGHDVDFIVTTTELGKEKNLLISVIESLKKQGLLLFSDYQASTFDISKLPSHRFEAMDHFAKCFLILRLEGSRVEGGLQRAQADGRGWRAVRVDLVSPPADRFAFTMLGWTGSRMFERDLRRFARLERQMLLDNHALYDKTKKEFLTAATEKDIFDHLGLEYIEPWQRNA

SEQ ID NO: 66 - protein, 366 aa, Artificial Sequence; identical to SEQ ID NO: 55
  F57 trunc new (Poecilia reticulata) guppy [56.66 percent identity to wt calc by NCBI BLASTP]

SEQ ID NO: 67 - protein, 362 aa, Rattus norvegicus
KISQYACQRRTTLNNHNQLFTDAFDILAENYEFRENEVSCLPFMRAASVLKSLSFPIVSMKDIEGIPCLGDKVKCVIEGIIEDGESSEVKAVLNDERYKSFKLFTSVFGVGLKTAEKWFRMGFRTLSKIKSDKSLRFTHMQKAGFLYYEDLVSCVNRAEAEAVSMLVKEAVVAFLPDALVTMTGGFRRGKMTGHDVDFLITSPEATEEEEQQLLHKVTNFWRQQGLLLYCDIIESTFEKFKLPSRKVDALDHFQKCFLILKLHRGLVRSEESGQQEGKDWKAIRVDLVMCPYERRAFALLGWTGSRQFERDLRRYATHERKMMLDNHALYDKTKRVFLEAESEEEIFAHLGLDYIEPWERNA

SEQ ID NO: 68 - protein, 366 aa, Artificial Sequence
  N100 trunc new
SVVQKISEYACQRRTTLNNHNEVFTRAFDILAENYEFRENEESRDAFRRASSVLKSLPFPIISMKDTEGIPCLGDKVKRVIEEIIEDGESSEVKAVLNDERYKAFKLFTSVFGVGRKTAEKWFRMGFRTLEKIKSDKSLRFTQMQKAGFLYYEDLAERVNRAEAEAIEMLVKEAVVAFLPDALVTMTGGFRLGKMTGHDVDFLITSPEATEEEEQQLLHKVTDFWRQQGLLLYCDIIESTFEKFKLPSRTVDALDHFQKCFLILKLHHGRVVSEKSGQQEGKGWKAIRVDLVMCPYERRAFALLGWTGSPFFNRDLRRYATHERKMMLDNHALYDKTKRVFLEAESEEEIFAHLGLDYIEPWERNA

SEQ ID NO: 69 - protein, 362 aa, Piliocolobus tephrosceles
KISQYACQRRTTLNNCNQIFTDAFDILAENCEFRENEDSCVTFMRAASVLKSLPFTIISMKDTEGIPCLGSKVKCIIEEIIEDGESSEVKAVLNDERYQSFKLFTSVFGVGLKTSEKWFRMGFRTLSKVRSDESLKFTRMQRAGFLYYEDLVSCVTRAEAEAVSALVKEAVWAFLPDAFVTMTGGFRRGKKMGHDVDFLITSPGSTEDEEQQLLQKVMNLWEKKGLLLYYDLVESTFEKLRLPSRKVDALDHFQKCFLIFKLPLQRVDSDQSSWQGGKTWKAIRVDLVMCPYERRAFALLGWTGSRQFERDLRRYATHERKMILDNHALYDKTKRIFLKAESEEEIFAHLGLDYIEPWERNA

SEQ ID NO: 70 - protein, 362 aa, Sus scrofa
KISQYACQRRTTLNNCNHIFTDAFEVLAENYEFRENETFCLAFMRAASVLKSLPFTIISMKDTEGIPCLGDKVKCVIEEIIEDGESSEVKAVLNDERYQSFKLFTSVFGVGLKTSERWFRMGFRSLSKIRSDKTLKFTRMQKAGFLYYEDLVSCVTRAEAEAVGVLVKEAVQAFLPDAFVTMTGGFRRGKKMGHDVDFLITSPGSTDDEEQQLLPKVVNLWEREGLLLYCDLVESTLEKSKLPSRNVDALDHFQKCFLILKLHHQRVDSGMSSQQEGKTWKAIRVDLVMCPYELRAFALLGWTGSRQFERDLRRYATHERKMILDNHALYDKTKRIFLKAESEEEIFAHLGLDYLEPWERNA

SEQ ID NO: 71 - protein, 362 aa, Artificial Sequence
  N27 trunc new  [88.13 percent identity to wt calc by NCBI BLASTP] pig
KISQYACQRRTTLNNYNEIFTRAFEILAENYEFRENEESYVTFRRAASVLKSLPFTIISMKDTEGIPCLGDKVKRVIEEIIEDGESSEVKAVLNDERYKAFKLFTSVFGVGRKTSEKWFRMGFRTLEKIRSDKTLKFTRMQKAGFLYYEDLVSRVTRAEAEAVSVLVKEAVWAFLPDAFVTMTGGFRLGKKIGHDVDFLITSPGSTEEEEQQLLHKVINLWEKKGLLLYYDLVESTFEKLKLPSRTVDALDHFQKCFLILKLHHQRVGSGKSSQQEGKTWKAIRVDLVMCPYERRAFALLGWTGSPQFNRDLRRYATHERKMMLDNHALYDKTKRIFLKAESEEEIFAHLGLDYIEPWERNA

SEQ ID NO: 72 - protein, 362 aa, Artificial Sequence
  N35 trunc new
KISQYACQRRTTLNNYNEIFTRAFEVLAENYEFRENEESYVTFRRAASVLKSLPFTIISMKDTEGIPCLGDKVKRVIEEIIEDGESSEVKAVLNDERYQAFKLFTSVFGVGRKTSEKWFRMGFRTLEKIRSDKTLKFTRMQKAGFLYYEDLAERVTRAEAEAVSVLVKEAVWAFLPDAFVTMTGGFRLGKKIGHDVDFLITSPGSTEEEEQQLLPKVINLWERKGLLLYYDLVESTFEKLKLPSRKVDALDHFQKCFLILKLHHQRVGSGKSSQQEGKTWKAIRVDLVMCPYERRAFALLGWTGSAFFNRDLRRYATHERKMILDNHALYDKTKRIFLKAESEEEIFAHLGLDYIEPWERNA

SEQ ID NO: 73 - protein, 361 aa, Bubalus bubalis
KISQYACQRKTTLNNYNHIFTDAFEILAENSEFKENEVSYVTFMRAASVLKSLPFTIISMKDTQGIPCLGDKVKCVIEEIIEDGESSEVKAVLNDERYQSFKLFTSVFGVGLKTSEKWFRMGFRSLSKITSDKTLKFTKMQKAGFLYYEDLVSCVTRAEAEAVGVLVKEAVWAFLPDAFITMTGGFRRGKKIGHDVDFLITSPGSAEDEEQLLPKVINLWEKKGLLLYYDLVESTFEKFKLPSRQVDTLDHFQKCFLILKLHHQRVDSGRSNQQEGKTWKAIRVDLVMCPYENRAFALLGWTGSRQFERDIRRYATHERKMMLDNHALYDKTKRVFLKAESEEEIFAHLGLDYIEPWERNA

SEQ ID NO: 74 - protein, 362 aa, Marmota flaviventris
TISQYACQRRTTLNNCNRVFTDAFDVLAENYEFRENESCSVVFMRAASVLKSLPFTIISMRDLEGIPCLEGKAKSIIEEIIEDGESSEVKAVLNDERYKSFKLFTSVFGVGLKTSEKWFRMGFRTLSKIRSDKSLKFTHMQKAGFLYYEDLVSCVTRAEAEAVSVLVKEAVWAFLPDAFITMTGGFRRGKNIGHDVDFLITSAEATEEEEQQLLHKVTNLWEKKGLLLYCDLVESTFEKLKTPSRKVDALDHFQKCFLILKLHHQRVDSDKASQQGGKNWKAIRVDLVMCPYERRAFALLGWTGSRQFERDLRRYATHERKMILDNHALYDKTKRIFLKAESEEEIFAHLGLDYIEPWERNA

SEQ ID NO: 75 - protein, 362 aa, Artificial Sequence
  Truncated Mus musculus TdT M53
KISQYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLAERVNRPEAEAIRMLVKEAVVTFLPDALVTMTGGFRLGKMTGHDVDFLITSPEATEDEEQQLLHKVTDFWKQQGLLLYCDILESTFEKFKQPSRTVDALDHFQKCFLILKLDHPRVHSVKSGQQEGKGWKAIRVDLVMCPYDRRAFALLGWTGSPQFNRDLRRYATHERKMMLDNHALYDKTKRVFLEAESEEEIFAHLGLDYIEPWERNA

SEQ ID NO: 76 - protein, 379 aa, Artificial Sequence
  N93  [95.15 percent identity to wt (Marmota flaviventris)calc by NCBI BLASTP]
SPSPVPGSQNVPAPAVQTISQYACQRRTTLNNYNRVFTDAFDVLAENYEFRENEESSVVFRRAASVLKSLPFTIISMRDLEGIPCLEGKAKRIIEEIIEDGESSEVKAVLNDERYKAFKLFTSVFGVGRKTSEKWFRMGFRTLEKIRSDKSLKFTHMQKAGFLYYEDLVSRVTRAEAEAVSVLVKEAVWAFLPDAFITMTGGFRLGKNIGHDVDFLITSAEATEEEEQQLLHKVTNLWEKKGLLLYCDLVESTFEKLKLPSRTVDALDHFQKCFLILKLHHQRVDSDKASQQGGKNWKAIRVDLVMCPYERRAFALLGWTGSPQFNRDLRRYATHERKMILDNHALYDKTKRIFLKAESEEEIFAHLGLDYIEPRERNA

SEQ ID NO: 77 - protein, 366 aa, Artificial Sequence
  N72 [91.76 percent identity to wt(Bubalus bubalis) calc by NCBI BLASTP]
SVVKKISQYACQRRTTLNNYNEIFTRAFEILAENYEFKENEESYVTFRRAASVLKSLPFTIISMKDTEGIPCLGDKVKRVIEEIIEDGESSEVKAVLNDERYQAFKLFTSVFGVGRKTSEKWFRMGFRSLEKIRSDKTLKFTRMQKAGFLYYEDLVSRVTRAEAEAVGVLVKEAVWAFLPDAFVTMTGGFRLGKKIGHDVDFLITSPGSTEDEEQQLLPKVINLWERKGLLLYYDLVESTFEKFKLPSRQVDALDHFQKCFLILKLHHQRVGSGKSSQQEGKTWKAIRVDLVMCPYEQRAFALLGWTGSAFFNRDLRRYATHERKMMLDNHALYDKTKRIFLKAESEEEIFAHLGLDYIEPRERNA

SEQ ID NO: 78 - protein, 379 aa, Artificial Sequence
  N35  trunc new [89.18 percent identity to wt (Panthera tigris altaica) calc by NCBI BLASTP]
SPSPVPGSQNVPAPVVKKISQYACQRRTTLNNYNEIFTRAFEVLAENYEFRENEESYVTFRRAASVLKSLPFTIISMKDTEGIPCLGDKVKRVIEEIIEDGESSEVKAVLNDERYQAFKLFTSVFGVGRKTSEKWFRMGFRTLEKIRSDKTLKFTRMQKAGFLYYEDLAERVTRAEAEAVSVLVKEAVWAFLPDAFVTMTGGFRLGKKIGHDVDFLITSPGSTEEEEQQLLPKVINLWERKGLLLYYDLVESTFEKLKLPSRKVDALDHFQKCFLILKLHHQRVGSGKSSQQEGKTWKAIRVDLVMCPYERRAFALLGWTGSAFFNRDLRRYATHERKMILDNHALYDKTKRIFLKAESEEEIFAHLGLDYIEPWERNA

SEQ ID NO: 79 - protein, 379 aa, Artificial Sequence
  N27 trunc new  [88.13 percent identity to wt(Sus scrofa) calc by NCBI BLASTP]
SPSPVPGSQNVPAPVVKKISQYACQRRTTLNNYNEIFTRAFEILAENYEFRENEESYVTFRRAASVLKSLPFTIISMKDTEGIPCLGDKVKRVIEEIIEDGESSEVKAVLNDERYKAFKLFTSVFGVGRKTSEKWFRMGFRTLEKIRSDKTLKFTRMQKAGFLYYEDLVSRVTRAEAEAVSVLVKEAVWAFLPDAFVTMTGGFRLGKKIGHDVDFLITSPGSTEEEEQQLLHKVINLWEKKGLLLYYDLVESTFEKLKLPSRTVDALDHFQKCFLILKLHHQRVGSGKSSQQEGKTWKAIRVDLVMCPYERRAFALLGWTGSPQFNRDLRRYATHERKMMLDNHALYDKTKRIFLKAESEEEIFAHLGLDYIEPWERNA

SEQ ID NO: 80 - protein, 379 aa, Artificial Sequence
  N14 trunc new  [92.08 percent identity to wt(Piliocolobus tephrosceles) calc by NCBI BLASTP]
SPSPVPGSQNVPAPVVQKISQYACQRRTTLNNYNEIFTRAFDILAENSEFRENEDSYVTFRRAASVLKSLPFTIISMKDTEGIPCLGSKVKRIIEEIIEDGESSEVKAVLNDERYQAFKLFTSVFGVGRKTSEKWFRMGFRTLEKVRSDESLKFTRMQRAGFLYYEDLVSRVTRAEAEAVSVLVKEAVWAFLPDAFVTMTGGFRLGKKMGHDVDFLITSPGSTEDEEQQLLQKVMNLWEKKGLLLYYDLVESTFEKLRLPSRTVDALDHFQKCFLIFKLPLQRVGSDQSSWQEGKTWKAIRVDLVMCPYERRAFALLGWTGSAFFNRDLRRYATHERKMILDNHALYDKTKRIFLKAESEEEIFAHLGLDYIEPWERNA

SEQ ID NO: 81 - protein, 379 aa, Artificial Sequence
  N100 or trunc new  [93.14 percent identity to wt(long Rattus norvegicus) calc by NCBI BLASTP]
SPSPVPGSQNVPAPVVQKISEYACQRRTTLNNHNELFTRAFDILAENYEFRENEESRDAFRRAASVLKSLSFPIVSMKDIEGIPCLGDKVKRIIEEIIEDGESSEVKAVLNDERYKAFKLFTSVFGVGRKTAEKWFRMGFRTLEKIRSDKSLRFTHMQKAGFLYYEDLVSRVNRAEAEAVSMLVKEAVVAFLPDALVTMTGGFRLGKMTGHDVDFLITSPEATEEEEQQLLHKVTNFWRQQGLLLYCDIIESTFEKFKLPSRTVDALDHFQKCFLILKLHRGLVRSEESGQQEGKDWKAIRVDLVMCPYERRAFALLGWTGSPFFNRDLRRYATHERKMMLDNHALYDKTKRVFLEAESEEEIFAHLGLDYIEPWERNA

SEQ ID NO: 82 - protein, 366 aa, Artificial Sequence; identical to SEQ ID NO: 68
  N100 trunc new  [89.86 percent identity to wt short Rattus norvegicus calc by NCBI BLASTP]

SEQ ID NO: 83 - protein, 383 aa, Artificial Sequence
  F57 trunc new (Poecilia reticulata)  [56.66 percent identity to wt calc by NCBI BLASTP]
SPSPVPGSQNVPAPSVDKISQYACQRRTTLNNYNKKFTDAFEILAENYEFNENKGFCLAFRRAASVLKFLPFTIVRMNDIQGLPWMGDQVKRVIEEILEEGESSKVKEVLNDEKYKAFKLFTSVFGVGRKTSEKWYRMGLRTLEEVKADKTLKLTKMQKAGFLYYEDLISRVSKAEADAVSLIVKDTVWKFLPDALVTLTGGFRLGKKMGHDVDFLITTPGPEEEEELLHKVIDLWKKQGLLLYYDIIESTFEKTKLPSRTVDALDHFQKCFAILKLHKQRVDNGNSNQSKEFDKEETKDWKAIRVDLVITPFEQYAYALLGWTGSAFFNRDLRRYATHEKKMMLDNHALYDKTKRIFLKAASEEEIFAHLGLDYLEPWERNA

SEQ ID NO: 84 - protein, 378 aa, Artificial Sequence
  F32 trunc new  [89.95 percent identity to wt Anabas testudineus calc by NCBI BLASTP]
SPSPVPGSQNVPAPSVATVSQYACQRRTTTENNNKIFTDAFEVLAESYEFNEMEESRDAFRRAASVLKSLPWAVQSLGATQDLPCLGEHTKRVMEEILQYGRSFEVEKILSDERYQTLKLFTSVFGVGPKTAEKWYRRGLRSFEDIRAEPSIHLNRMQQSGFLHYGDISRAVSKAEARALGNIIDEAVHAITPDAILALTGGFRLGKEFGHDVDFIVTTPELGKEESLLPNIIDRLKKQGILLYSDYQASTFDMSKLPSHRFEAMDHFAKCFLILRLEGSQVEGGLNSAEGDSRGWRAVRVDLVSPPMDRYAFALLGWTGSAFFNRDLRRFARMERRMLLDNHALYDKTKKEFLAATTEKDIFAHLGLEYIEPWQRNA

SEQ ID NO: 85 - protein, 377 aa, Artificial Sequence
  F24 trunc new  [83.55 percent identity to wt Electric eel calc by NCBI BLASTP]
SPSPVPGSQNVPAPSLPTVSQYACQRRTTLDNHNKIFTDALEVLAENYEFSDSKESRDAFRRAASVLKSLPTPLRSLQDTEGLPCLGEETKRVIEEIFEEGSSSRVEDILSDERYQTLKLFTSVFGVGPKTAEKWYRRGLRSLEQVRSDPSIHLNRMQTAGFLYYEDISKPVSKAEAKALGNIIEETASAFSPSVTITLTGGFRLGKEFGHDVDFLLTVPEPGKEDGLLPAVIDRLRSQGILLYSDFQESTFDLSKLPSRRFEAMDHFQKCFLIVKLKAGLVEGEGADPGDRRDWRAVRVDLVAPPVDRYAFALLGWSGSAFFNRDLRRFARLERGMLLDNHALYDKTTNTFLQAKTEEDIFAHLGLDYIEPWQRNA

SEQ ID NO: 86 - protein, 367 aa, Artificial Sequence
  F15 trunc new  [80.38 percent identity to wt Salmo truttal calc by NCBI BLASTP]
SSVATVSQYACQRRTTMENHNKIFTDAFEVLAENSEFNESKESRDAFRRAASVLKSLPSAVRSLGDTEGLPCLGEHTKRVIEEILEYGRSSKVEDILSDERYQTMKLFTSVFGVGPKTAEKWYRRGLRSLEEVRAEPSIHLNRMQRAGFLYYRDISKRVSKAEAKALGNIIEETVHAIAPDAIVTLTGGFRLGKEFGHDVDFILTTPEMGKEEGLLPRVIDRLRNQGILLYCDYQESTFDMSKLPSRTFEAMDHFQKCFLIIKLKEGLVEGEGGLQSDPGDRRGWRAVRVDLVAPPVDRYAFALLGWTGSPQFNRDLRRFARLERRMLLDNHALYDKTKKIFLPAKTEEDIFAHLGLEYIEPWQRNA

SEQ ID NO: 87 - protein, 384 aa, Artificial Sequence
  B63 trunc new  [85.68 percent identity to wt Notechis scutatus calc by NCBI BLASTP]
SPSPVPGSQNVPAPSVDSISQYACQRRTTLNNYNKKFTDAFEILAENYEFNENKGFCLAFRRAASVLKFLPFTIVRVNDIEGLPWMGEQVKRIIEDILEEGESSKVKAVLNNENYRAFKLFTSVFGVGRKTSEKWYRMGLRTLEEVKADKNLKLTRMQKAGFLHYEDLISRVSKAEADAASLIVKDTVWKFLPNALVTLTGGFRLGKKMGHDVDFLITVPGSRQEEEELLHTVIDLWKKQGLLLYYDLIESTFEKTKLPSRTVDALDHFQKCFAILKLHKERVDKGNSIQSKAFAEEETKDWKAIRVDLVVTPFEQYAFALLGWTGSPFFNRDLRRYATHEKKMMLDNHALYDKTKKIFLKAASEEEIFAHLGLDYLEPWERNA

SEQ ID NO: 88 - protein, 380 aa, Artificial Sequence
  B59 trunc new [84.74 percent identity to wt xenopus calc by NCBI BLASTP]
SPSPVPGSQNVPAPSEVKVSQYACQRRTTLQDTNRIFTDAFDILAENYEFNENKGPCVAFRRASSVLKSLPFPIVAMKDLEGLPWLGDQMKRIIEEILEEGKSSKVVEVMNDERYKAFKLFTSVFGVGRKTSEKWFRMGFRTLEEIKSDKELKLTKMQKAGLLYYEDITSAVSKAEAEAVEQLIKSIVWKFVPDAIVTLTGGFRLGKKMGHDVDILITTPRKGEKEEILHKTINVLKKRGLLLFYNIIESTFDETKLPSRHVDALDHFQKCFTILKLQKQQLDNGNSNEPFETEKKNWKAIRVDLVITPYEQYAYALLGWTGSPQFNRDLRRYATHEKRMMLDNHALYDKTKNIFLKANSEEDIFTHLGLDYLEPWERNA

SEQ ID NO: 89 - protein, 383 aa, Artificial Sequence; identical to SEQ ID NO: 83
  B57 trunc new [83.03 percent identity to wt alligator calc by NCBI BLASTP; 84.07 % id to purple]

SEQ ID NO: 90 - protein, 370 aa, Artificial Sequence
  B19 trunc new [92.35 percent identity to wt neopelma calc by NCBI BLASTP]
SSVSKVSQYSCQRKTTLNNYNKKFTDAFEIMAENYEFKENEIFCLEFRRAASVLKFLPFPVTRMKDIQGLPCMGDRVRDVIEEIIEEGESSRVKEVLNDERYKAFKQFTSVFGVGRKTSEKWYRMGLRTLEEVKADKTLKLSKMQKAGFLYYEDLAERVSKAEADAVSLIVKNTVVTFLPDALVTITGGFRLGKKIGHDIDFLITNPGPREDEELLHKVVDLWKKQGLLLYCDIIESTFVKEQLPSRKVDAMDNFQKCFAILKLYQPRVDNSSYNTSKKFDMAEVKDWKAIRVDLVITPFEQYAYALLGWTGSPQFNRDLRRYATHERKMILDNHALYDRRKRIFLKAGSEEEIFAHLGLDYVEPRERNA

SEQ ID NO: 91 - protein, 369 aa, Artificial Sequence
  specific Bos taurus from Misha
KTPPLVVKKISQYACQRKTTLNNYNEIFTRAFEILAENSEFKENEESYVTFRRAASVLKSLPFTIISMKDTEGIPCLGDKVKRIIEEIIEDGESSEVKAVLNDERYQAFKLFTSVFGVGRKTSEKWFRMGFRSLEKIRSDKTLKFTKMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSAFFNRDIRRYATHERKMMLDNHALYDKTKRVFLKAESEEEIFAHLGLDYIEPRERNA

SEQ ID NO: 92 - protein, 374 aa, Artificial Sequence
  specific latmeria from Misha
NVPAPSVVAISQYACQRRTTLNNHNKIFTDAFEILAENYEFNENEGPCLAFRRAASLLKSLPYAISSMKDLEGLPCLGDQTKAVIEEILEEGQSSKVQDVLSDERYKSIKLFTSVFGVGLKTAEKWYRKGFRTLEEVQADKEIKLTKMQKAGFLYYEDISSAVTKAEAEAIGQIIEDTVRLFAPDAIVTLTGGFRLGKKIGHDVDFLITTPETGNENGLLHKVINVLQNQGILLYYDIVESTFDKTRLPSRKVDALDHFQKCFAILKLHKQKVNTSNSEEAEEPSNTETKDWKAIRVDLVITPFDQYAFALLGWTGSAFFNRDLRRFATHERKMMLDNHALYDKTKKIFLPAKTEEDIFAHLGLDYIEPWERNA

SEQ ID NO: 93 - protein, 370 aa, Artificial Sequence
  specific Puma concolor from Misha
KTESAVVKKIPLYACQRRTTLNNFNEIFTRAFEVLAENYEFKENEISSATFRRAASVLKSLPFTIISMKDTEGIPCLGDKVKRVIEEIIEDGESSEVKAVLNDERYQAFKLFTSVFGVGLKTSEKWFRMGFRTLEKIKSDKTLKFTQMQKAGFLYYEDLVSGVTRAEAEAVGVLVKEAVWAFLPDAFVTMTGGFRLGKKIGHDVDFLITIPGSTDEEEEQLLPKVINLWQRKELLLYYDLVESTFEKLKLPSRKVDALDHFQKCFLILKLHHQRVDSGKCSQQEGKTWKAIRVDLVMCPYERRAFALLGWTGSAFFNRDLRRYATHERKMILDNHALYDKTKKIFLKAESEEEIFAHLGLDYIEPWERNA

SEQ ID NO: 94 - protein, 370 aa, Artificial Sequence; identical to SEQ ID NO: 93
  specific N139 from Misha

SEQ ID NO: 95 - protein, 370 aa, Artificial Sequence
  specific Elephantulus edwardii from Misha
NVPAPVVQKISQYACQRRTTLNNHNHIFTDAFEILAENDEFRENEESRDAFRRAASVLKSLPFSIISMKDTEGIPCLADKVKRVIEEIIEDGESSEVKAVLNDERYKAFKLFTSVFGVGRKTAEKWFRLGFRTLEGIRNDKTLKLTHMQKAGFLYYEDLVSGVTRAEAEAVGVLVKEAVWAFLPDAIVTMTGGFRLGKKVGHDVDFLITSPEATEEQEQQLLHKVITFWEKEGLLLYCDLYESTFEKLKMPSRTVDALDHFQKCFLILKLHRESVDDGTSSQLQGKTWKAIRVDLVVCPYECRAFALLGWTGSPFFNRDLRRYATHERKMMLDNHALYDKTKRKFLSADSEEDIFAHLGLDYIEPRERNA

SEQ ID NO: 96 - protein, 381 aa, Artificial Sequence
  M27
NSSPSPVPGSQNVPAPVVKKISQYACQRRTTLNNYNQLFTDALDILAENDEFRENEESCLAFRRASSVLKSLPFPITSMKDTEGIPCLGDKVKSIIEGIIEDGESSEVKAVLNDERYKSFKLFTSVFGVGLKTAEKWFRMGFRTLSKIQSDKSLRFTQMQKAGFLYYEDLVSGVNRPEAEAVSMLVKEAVVTFLPDALVTMTGGFRLGKMTGHDVDFLITSPEATEDEEQQLLHKVTDFWKQQGLLLYCDILESTFEKFKQPSRTVDALDHFQKCFLILKLDHPRVHSVKSGQQEGKGWKAIRVDLVMCPYDRRAFALLGWTGSPQFNRDLRRYATHERKMMLDNHALYDKTKRVFLEAESEEEIFAHLGLDYIEPWERNA

SEQ ID NO: 97 - protein, 383 aa, Artificial Sequence
  B10 trunc new [91.15 percent identity to wt canary calc by NCBI BLASTP]
SPSPVPGSQNVPAPSVTKVSQYSCQRKTTLNNYNKKFTDAFEVMAENYEFKENEESRDAFRRAASLLKSLPFPVTRMKDIQGLPCMGDQVRRIIEEIIEEGESSRVKEVLNDERYKAFKQFTSVFGVGRKTSEKWYRMGLRTVEEVKADKTLKLSKMQKAGFLYYEDLVSRVSKAEADAVSLIVKNTVVTFLPDALVTITGGFRLGKKIGHDIDFLITNPGPREDEELLHKVIDLWKKQGLLLYCDIIESTFVKEQLPSRKVDAMDHFQKCFAILKLYQPRVDNSTSNTSKKLEMAEVKDWKAIRVDLVITPFEQYAYALLGWTGSAFFNRDLRRYASHERKMILDNHALYDRRKRIFLKAGSEEEIFAHLGLDYVEPWERNA

SEQ ID NO: 98 - protein, 381 aa, Artificial Sequence
  Mus musculus TdT mutant  >TH M27
SGSPSPVPGSQNVPAPVVKKISQYACQRRTTLNNYNQLFTDALDILAENDEFRENEESCLAFRRASSVLKSLPFPITSMKDTEGIPCLGDKVKSIIEGIIEDGESSEVKAVLNDERYKSFKLFTSVFGVGLKTAEKWFRMGFRTLSKIQSDKSLRFTQMQKAGFLYYEDLVSGVNRPEAEAVSMLVKEAVVTFLPDALVTMTGGFRLGKMTGHDVDFLITSPEATEDEEQQLLHKVTDFWKQQGLLLYCDILESTFEKFKQPSRTVDALDHFQKCFLILKLDHPRVHSVKSGQQEGKGWKAIRVDLVMCPYDRRAFALLGWTGSPQFNRDLRRYATHERKMMLDNHALYDKTKRVFLEAESEEEIFAHLGLDYIEPWERNA

SEQ ID NO: 99 - protein, 381 aa, Artificial Sequence
  Mus musculus TdT mutant >MS 13-34
SGSPSPVPGSQNVPAPVVKKISQYACQRRTTLNNYNELFTRALDILAENDEFRENEESRDAFRRASSVLKSLPFPITSMKDTEGIPCLGDKVKRIIEEIIEDGESSEVKAVLNDERYQAFKLFTSVFGVGRKTAEKWFRMGFRTLEKIRSDKSLRFTQMQKAGFLYYEDLVSGVNRPEAEAVSMLVKEAVVTFLPDALVTMTGGFRLGKMTGHDVDFLITSPEATEDEEQQLLHKVTDFWKQQGLLLYCDILESTFEKFKQPSRTVDALDHFQKCFLILKLDHPRVHSVKSGQQEGKGWKAIRVDLVMCPYDRRAFALLGWTGSAFFNRDLRRYATHERKMMLDNHALYDKTKRVFLEAESEEEIFAHLGLDYIEPRERNA

SEQ ID NO: 100 - protein, 381 aa, Artificial Sequence
  Mus musculus TdT mutant >MS 34-1
SGSPSPVPGSQNVPAPVVKKISQYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLVSGVNRPEAEAVSMLVKEAVVTFLPDALVTMTGGFRLGKMTGHDVDFLITSPEATEDEEQQLLHKVTDFWKQQGLLLYCDILESTFEKFKQPSRTVDALDHFQKCFLILKLDHPRVHSVKSGQQEGKGWKAIRVDLVMCPYDRRAFALLGWTGSAFFNRDLRRYATHERKMMLDNHALYDKTKRVFLEAESEEEIFAHLGLDYIEPWERNA

SEQ ID NO: 101 - protein, 381 aa, Artificial Sequence
  Mus musculus TdT mutant >TH c2_5
SGSPSPVPGSQNVPAPVVKKISQYACQRRTTLNNYNELFTRALDILAENDEFRENEESCLAFRRASSVLKSLPFPITSMKDTEGIPCLGDKVKRIIEEIIEDGESSEVKAVLNDERYKSFKLFTSVFGVGLKTAEKWFRMGFRTLEKIRSDKSLRFTQMQKAGFLYYEDLVSGVNRPEAEAVSTLVKEAVVTFLPDALVTMTGGFRLGHMTGHDVDFLITSPEATEDEEQQLLHKVTDFWKQQGLLLYCDILESTFEKFKQPSRKVDALDHFQKCFLILKLDHLRVHSAKSGQQEGKGWKAIRVDLVMCPYDRRAFALLGWTGSVQFKRDLRRYATHERKMMLDEHALYDKTKRVFLEAESEEEIFAHLGLDYIEPWERNA

SEQ ID NO: 102 - protein, 361 aa, Artificial Sequence
  (M57 without His tag & linker & with Q4E mutation) Bos taurus-Mus musculus chimera
KISEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 103 - protein, 361 aa, Artificial Sequence
  (M59 without His Tag and linker & with Q4E mutation) Bos taurus-Mus musculus chimera
KISEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLAERVNRPEAEAIRMLVKEAVVTFLPDALVTMTGGFRLGKMTGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 104 - protein, 362 aa, Artificial Sequence
  (M27 with maximal N-terminal deletion plus Q4E mutation) Mus musculus
KISEYACQRRTTLNNYNQLFTDALDILAENDEFRENEESCLAFRRASSVLKSLPFPITSMKDTEGIPCLGDKVKSIIEGIIEDGESSEVKAVLNDERYKSFKLFTSVFGVGLKTAEKWFRMGFRTLSKIQSDKSLRFTQMQKAGFLYYEDLVSGVNRPEAEAVSMLVKEAVVTFLPDALVTMTGGFRLGKMTGHDVDFLITSPEATEDEEQQLLHKVTDFWKQQGLLLYCDILESTFEKFKQPSRTVDALDHFQKCFLILKLDHPRVHSVKSGQQEGKGWKAIRVDLVMCPYDRRAFALLGWTGSPQFNRDLRRYATHERKMMLDNHALYDKTKRVFLEAESEEEIFAHLGLDYIEPWERNA

SEQ ID NO: 105 - protein, 361 aa, Artificial Sequence
  M54 with maximal N-terminal deletion plus Q4E mutation
KISEYACQRKTTLNNYNEIFTRAFEILAENSEFKENEESYVTFRRAASVLKSLPFTIISMKDTEGIPCLGDKVKRIIEEIIEDGESSEVKAVLNDERYQAFKLFTSVFGVGRKTSEKWFRMGFRSLEKIRSDKTLKFTKMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSAFFNRDIRRYATHERKMMLDNHALYDKTKRVFLKAESEEEIFAHLGLDYIEPRERNA

SEQ ID NO: 106 - protein, 362 aa, Artificial Sequence
  M77 without His tag
KKISEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 107 - protein, 361 aa, Artificial Sequence; identical to SEQ ID NO: 102
  C1-40 without His tag

SEQ ID NO: 108 - protein, 363 aa, Artificial Sequence
  A2-20 without His tag
VTTVSEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 109 - protein, 363 aa, Artificial Sequence
  ii-ell-40 without His tag
VATVSEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 110 - protein, 363 aa, Artificial Sequence; identical to SEQ ID NO: 109
  9-40 without His tag

SEQ ID NO: 111 - protein, 365 aa, Artificial Sequence
  (45-751)(internal His tag)
MAEKKISEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEEIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTLEKFKLPSRQVDTLDHFQKCFLILKLHRQRVDSSKHHHHHHKTWKAIRVDLVMCPYENRAFALLGWTGSPQFNRDLRRYATHERKMMLDNHALYDKTKRVFLEAESEEEIFAHLGLDYIEPWERNA

SEQ ID NO: 112 - protein, 369 aa, Artificial Sequence
  (46-737) (internal His tag)
MAHHGSEKKISEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEEIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTLEKFKRPSRQVDTLDHFQKCFLILKLHRQRVDSSKHHHHHHETWKAIRVDLVMCPYENRAFALLGWTGSPQFNRDLRRYATHERKMMLDNHALYDKTKRVFLEAESEEEIFAHLGLDYIEPWERNA

SEQ ID NO: 113 - protein, 552 aa, Saccharomyces cerevisiae
MASQKVFGITGPVSTVGATAAENKLNDSLIQELKKEGSFETEQETANRVQVLKILQELAQRFVYEVSKKKNMSDGMARDAGGKIFTYGSYRLGVHGPGSDIDTLVVVPKHVTREDFFTVFDSLLRERKELDEIAPVPDAFVPIIKIKFSGISIDLICARLDQPQVPLSLTLSDKNLLRNLDEKDLRALNGTRVTDEILELVPKPNVFRIALRAIKLWAQRRAVYANIFGFPGGVAWAMLVARICQLYPNACSAVILNRFFIILSEWNWPQPVILKPIEDGPLQVRVWNPKIYAQDRSHRMPVITPAYPSMCATHNITESTKKVILQEFVRGVQITNDIFSNKKSWANLFEKNDFFFRYKFYLEITAYTRGSDEQHLKWSGLVESKVRLLVMKLEVLAGIKIAHPFTKPFESSYCCPTEDDYEMIQDKYGSHKTETALNALKLVTDENKEEESIKDAPKAYLSTMYIGLDFNIENKKEKVDIHIPCTEFVNLCRSFNEDYGDHKVFNLALRFVKGYDLPDEVFDENEKRPSKKSKRKNLSSGENLYFQGSSGS

SEQ ID NO: 114 - protein, 674 aa, Myceliophthora thermophila
MAERIYGVTPPISTALPTEEEKRLNNALHQELRAQGTFESPAETEKRKEVLRQLEKITNVFVQRAAAEKEPKNTILIRDAIGRVFTYGSYRLGVYGPGSDMDTLVVAPKYVTVEQYFRIFPEVLVEMAPPGAITDLTPVPEAFVPIIKFEFSGISIDLIFCSIQTLKQLPADKNWSLADNNLLRGLSENEVRSLNGTRVTDDILNLVPEPATFKLALRAIKLWAQRKAIYANIMGYPGGVAWAMLVARVCQLYPKATSAVIVNKFFHIMLKWPWPLPVLLKDIEYGCPVTRVAVWNPKIYASDRNHRMPIITPSYPSMCATHNVGRSSMAVIKDELEKGVQVTEDIMRGKRPWKDLFTKHTFFTSGFRYYLTVISSSRTKKAQNVWSGFVESRVRLLVNKLEMHPSISLARPFNKGYDREHRCKNNAQLEEVVSLGSLAYMYKPATGEAKAEAKKEVKTEVKHESGASVKNEAPEAKGEDGVRAKRENGDEAQLPPATGIKPEPTDGAGADVKLEDIPVKKDEPEEMTIYTTNHYIGLQLVEGAKTLDLSREVNDWKAMCTSNELYEEGTMFLSIQHVRNTALPDDVFEPGETRPRPAKKSLKRVASEDPGNKQTQPPAKKQVQDKAPPAAQQQQQQQHQQQQQPASTAAAAAGSSGENLYFQGSSGSHHHHHH

SEQ ID NO: 115 - protein, 672 aa, Thermothielavioides sp.
MAERTYGVTPPISTALPTEQEKALNKALHDELRAQGTFESRAETEKRKEVLAQLEKITNAFVQRAAREKHGKNAILIRDAIGRVFTYGSYRLGVYGPGSDMDTLVVAPKYVTVEQYFRIFPEVLVEMAPPAAITDLTPVPEAFVPIIKFEFSGISIDLIFCSIQTLIQLPADKSWSLADNNLLRGLSENAVRSLNGTRVTDEILHLVPEPATFKLALRAIKLWAQRKAIYANIMGYPGGVAWAMLVARVCQLYPKATSAVIVNKFFNIMLKWPWPLPVLLKDIEYNGPVTRVPVWNPKLYASDRNHKMPIITPAYPSMCATHNVGRSSMVVIQQELKKGAEVTEEIMLGRRPWKDLFTKHTFFTSGFKYYLTVISSSRTKKAQNVWSGFIESRVRLLVNKIEMHPSIALARPFNKGYDRMHRCKNDAQVEEVVSAGSLAYVYTPPAFGDEKVKSETKSEVKQEVKQEVRQDDVIQDGVPVKQEKAEVRAEDGVRIKRELSEEVQLPPPTNVKPEPHDDEAKVKLEDIPEKEAQPEDMEIYTTNHYIGLQLVEGAKSLDLSREVNDWKAMCMSNELYEEGLMFLSIQHLKNTALPDDVFEPGEVKPRPGKKVLKRGASEEPSKQQPPAKRQAHVQPRAPAAQQPSSTATAAAGSSGENLYFQGSSGSHHHHHH

SEQ ID NO: 116 - protein, 566 aa, Pyronema omphalodes
MSAPPKQYGTTPPINTNPPTKEDMERNSELVQELKNQNCFEEKAESDKRVKVLEILQTLGEAFVKKACAAKGLPDHLVNNSGGKIFTFGSYRLGAYGPGSDIDTLLVVPAHIERADFFQHVPKMLSELNPPAEEVSPVPDAFVPIIKFELQSISIDLIFAKVPSVNSIPRDMTLENKDILKGCDEANLRGLNGVRLTDELLGLVPHPVNFRMALRAIKLWAKSRAIYANVMGFPGGIAWAMLVAKICQLYPMAVSAVIVCKFFTIYTKWKWPQPVLLKDILDEGGQGAGLRVWNPKIYASDRGHLMPVITPNYPCMCSTHNITKSTKAVILREMDRSLNIIHEIMDGKSPWSKLFAPHTFFTQDYRYYLRVISAARTKDEQLLWSGLVESKLRHLVSKLEMLDNIQLAHPFNKGFEYEVECNNEDEVMRVIRGENIDQSEANGTAVDSNGKSSKVKAYTTSFYVGLVLDTSKSKQFDISWACHEFYDICKTWNLYNDDMHSIHVVNTRNFELPDNVFKPGETKPTKKTKTIKRKIPNAAGKKRGADEISAPDSKRAHINEVPPNAN

SEQ ID NO: 117 - protein, 598 aa, Tilletia indica
MTKHLGVTPIVSDASPTAHEIALETQLVTELTTQNCFESPQDSRLREVVLGKADKLVKEFVYRAAIARGLPESAARELGGKIFTFGSYRLGVHGPGSDIDTLCVVPKHVQREDFFTIFEQMLRQREDVTDVAPVPEAYVPLIAVNFMGIAIDFLFARLSLSRIDDSLDLSDNTILKNMDDQDIRSVGGSRVTDEILRLVPNVETFHTALRAIKLWAQRRAIYKNMVGFPGGVAWAMLVARICQLYPNGNAAVIIERFFIIMFQWNWPQPVMLKHHIYEADPILKVWNPRLYAADRSHRMPIITPAYPGMCATHNITASTQAVLTLEFKRGIEIMSAMSASITNPALPQTTWATLFESRRFFEEYKYYLQIIASSGSADLQLKWAGTVESKLRHLVLSTEAQPGVKIAHPYTREIDLVSECHTDEEVRRVATGDVPPEVAARARTVEGEGVLPESKEVDKLKEIESKEEEARKKGRRTIWTTTFYIGLALHDGSDDKTGQSAPKRLDLVGPIQDFKGRCTEWATYDESTMGVVVRHVKRSQLPDSVFPDGKRPAAPVKKVVKKRKRNAGMAEAAAETKEEEGADEQEAKRARTNGDSAA

SEQ ID NO: 118 - protein, 574 aa, Clathrospora elynae
MNGPPKTQYGVTSAISEAAPTEEDRLLNDKLIETLKRENVFETPEGNAKREEVILHLQKVVEEFVRRVGKQKGVPQSTIDAAGGKVFTFGSYALGVHGPSSDIDTLVVAPKFVTIDEFFQTFPPTFKEMSRVEDIKEFVPVEDAFVPIIKMEYRGVSIDLLFASLPRMASIPKDMDTIDKKNLEGLGESATRSVNGTRVTKELLAAVPQNNSFRHALRAIKLWSSRRGIYGAVFGYPGGVAWAIMVARICQLYPFANGATIVSKFFSLMYKWTWPRPVMLKHIEEGAMGLRVWNPQIYGGDRAHLMPIITPAFPSMCATHTVMPSTLRIMKEEFGRADKILQHVFAGTKKWDALFERHSFFTKDHKYYLSVVAASRTKEANSTFSGLVQSKIRHIVKGIDDGQTGVDIARPYIDYFERYHRCKDEDQIFQVCQGKLDHMIPASELPAEGTAPANGDSHIMYTTTFYIGLTLPLGTATEGTKSLDISYPVSQFKNFITDADKFDKETMSVKVVHTRSSALPDDVFVQGETRPKKPVKEKKKKEAKSAKRQFADTGLDVRDSRLTKPSQRSSASRE

SEQ ID NO: 119 - protein, 546 aa, Drechslerella brochopaga
MPASVDVLKRLQKITEEFVRQVYTSKNQNELTTNSAGGKVFTYGSYRLGVVGPGSDIDTLVVAPKLVTREDFFKFYPPLLKALNKANEPPVIEELAPVPDAFVPIIKFVMSGISIDLIFCRLGVAQVPADMTLEDKNLLRGLDEKELRSLNGTRVTDEILTLVPVPAVFKHALRAIKIWAKCRAIYGNVYGFPGGVAWAMLVARICQLYPSAVSAVIVSKFFRILGQWNWPQPVLLKPIEDGPLNVRVWNPKLYPSDRNHLMPIITPAYPSMCSTHNITPSTKAVILGEMAKAADIVDRIITGNGNWNQLFQRHTFFTKDYKYYLTVKASARDQEQSVKWSGLVESKIRHLVMKLEMLSDVIASARPYVKPFEKVQYCRNQEEAHKIAMGQHVPDSPPAEAANAATGETAGEVSEQEKEGGVPVWITTFHIGIELTPGMVGQVNISWPAQEFYIMCHNWEGYEENMHCVKLELLRNWNLPDECFDFSKGDAKPSRPIVKRKVIRKVVSSSEVSQSGMKRTHAEENPNADEGDVKRTKVNAPVTVQG

SEQ ID NO: 120 - protein, 632 aa, Magnaporthiopsis poae
MAERQLGVTPPVSVSLPSEFELQATDALLAELRAQNSFESPLETQKRHRVLASLQTIADAFVKVVAQEREPHNAVLIKDARAKVFAYGSLRLGVWGPGSDIDTLVVGPRYVTREDYFKHFPDLLVKMAPPGAITDLAVVQEAFVPIIKFEYHGISIDLIFSRIATLKQLPSDPAWDLKDNNLLRGLDEKELRSVNGTRVTDEILSLVPEQNIFRTALRAIKLWAQRRAVYGNIMGFPGGVAWAMLVARVCQLYPKATSSVIVNKFYSILLQWPWPQPVLLKPIGDGPLQVRVWNPRLYKGDSYHLMPVITPAYPSMCATFNVTRSSMTVIYRELQLAAEVTNNIMCSTKPWKDLFTKHTFFTKDFKYYMQVISASRDKEAHKIWSGFVESKVRMLVQSLERHDSIAVARPFIKGFERFHRYRNEEQFAQILDGNLQHMVKAGGDASPSQDGIQAKKEDNSSVKAEEDAKVKQENGETPVKQENGVKVKGEESEEAPLASIPEHSPVDTKGDSKAGDEALEMYTTTHYIGLELNPAAKSLDLSFQVNDFRALCHNWEKYKDELSDRCYVNIKDLRNFALPEDLFEPGEVKPVKSVKKRPAAGANATGDKKRPANEDNSNPAKRRQAASVAAAG

SEQ ID NO: 121 - protein, 631 aa, Cryptococcus depauperatus
MLGVTPPISIEAPKPGDIHSSEALMTDLIAMNQFESDSERKIREKLLSNIAQLVVKFVHDVSLKIGMSEKMASESGGRIYTSGSYRLGVHGPGSDIDTICVCPRHIYREHFFGEFQEMLRAWPEVTEITAVTGAFVPVMKTVISGVEVDLLFARVNLPEAGDSLDIEKDEILRGVDDASQRSLNGPRVTDMILNLVPDVTTFRTALRSIRLWARRRGIYSNVLGFPGGVAWALLTARICQLYPNATPSIIVGKFFPIYYQWNWPQPVILKKIENGPPNMQHAVWNPKLDRRDMAHRMPVITPAYPSMCSTHNITASTMSIISKEMLRAMQITDDILREPGASWAPLFEKVDFFSMYKTYVQVVASASTAEGIKDWSGTVEARIRTLVQDLENTDSILTAHPLVGGVSRVFYCTTEEEQAAASQGELTSEMIDRTEEEVTDKEHRNIFTKSFFIGLEIEKKTKEGGGRVLNLFYPSKKFCSMCQSWDRYNEMEMSVILRPAKRSDLPSFVFPDGIPISKKKAKRQQQNGSADASLSDSVEGQGPSKRTKAAPPSPNQASNPRCNGLPVQNGSDPVSSKIGEEVDIKPPPGIENLPPLSNAAMSSFATAAKGVATTQDFNKEGLVVLNQQSTL

SEQ ID NO: 122 - protein, 571 aa, Golovinomyces cichoracearum
MAEKSFGVTAPLSVTPPTESENQASSALIEELKRQNNYENAIETANRQKVLNSLQLITEEFVRQVSRAQGLPANLIKSAGGMVVTFGSYKLGVIGPGSDIDTLIVAPQNVTKEDFFSRFPDLLRSMATEGNITELTAKPDAFAPCITLKYAGIDIDLLFGRVKLSQVPRNLSLLNQNMLRGLNNEEVRSLNGVRVADEILNLVPEPAIFRTALRTIKLWGTRRAIAGNIYGFPGGVTWAILVARICQLYPKATSSTIVFKFFRIMEKWRWPMPVLLKEIDNLNALGLKVWNPKIYGSDKNHIMPIITPAYPEMCTTHNFSLSTKAILEKELKRGGDITDRVMSGKASWKDLFAKHTFFTNGYKYYLSVVSASRNKESQLFWSGFVESKVRLLVNKLEYHPSIALAHPFNKGFTRVHRCRTEEEVDLVKNGSLKFHATDIATSTTGHGLSVETTSKEKLNETKNVEQDENLIMVYTATHYIGLELAKGAKSLDLSYEVEEFKSICTSSEAYNQEENSLGVAHTKNCDLPDDVFTEGELKPTRPLKQKKKRPATEDSNPVPSKRQQTSLIAVG

SEQ ID NO: 123 - protein, 600 aa, Hortaea werneckii
MAEKQYGVTPAFSLEPPSPKDLKLNDALLAEFKAQNNFAPQSDTEKREAILKKLEGLLQRMVQEVGRKKGLPQSILEVAGGKVFTYGSYRLGVYGPNSDVDTLMVGPKHVTREDFFEHMPPLIRSAWAEDQIGGLVPVPGIGTPIIKLELEGVDIDLIYSSLQLSSIPKDIELKDDNLLRGLDDTDRRCVNGTRVTNRILELVPQTKTFRLALRAIKLWSSQRAIYGNIVGFPGGVAWAILVARVCQLYPKAAAPLLISKFFFIMKRWNWPKPVFLQHKEETSLQLREWDPIQYRGDGFHLMPILTPAYPSMNTAHTVGPSTKMIIMRELERGENIVNDIYANKRPWKDLFQRHTFFTNAYKHYICVVVAAKNKDAHDNWSGLVNSKLKFLVKGIEDSGGSSVELVQPFNKGFSRVHECQTNEQVEKVLDGSLECQVKETKTTEEGNDTAIANGVAQTDTEGLEVPKADSEARAEGSGTTKLWTTTFYLGIGLKKGATDLDISVPVRNLQGDCTAWADYNPDLHSIKIKHIRNFNLPDDVFKEGEVKPQRPKKKTNAAKAAEATSNKRSFSDTGLDPNADPAKRRQSGNVPTPVANGSVG

SEQ ID NO: 124 - protein, 589 aa, Valsa sordida
MAEIQYGMTPPLSTELPKDPEKRANDALFAELKAQNTYEHATETQKREKVLKSLQSIADVFVQKVAQRVHKESPAIQKSARGRVFTYGSYRLGVFGPGSDIDTLVVAPKYVTREDFFELFPGLLKELAPAGSITDLTAVTDAFVPIIKFEYSDISIDLIFSRIATLTEIPALTTKWDLLDNNLLRGLDDAELRSLNGTRVTDDILKLVPEQTSFRLALRAIKLWAQRRAIYANIMGFPGGVAWAMLVARICQLYPKANGAVIVDKFFHIIRRWPWPQPVLLKNPEDGPLQVRVWNPKVYKGDSYHLMPVITPAYPSMCSTFNVTHSNKAVIQKELDHFADVVQQIMMGKLPWKSLFVKHTFFTKDYKYYIAVIAASTTKENSKIWGGFVESKIRLLVQGLERHSSIRLARPFNKGYERKHRTVSGMNEWHDILDGSLDYVVKDEQVADEKAKTEPGNDAVKTEPTSPAKVKQEEGIESLEIKDDAKETDGKNEQDEEDAEVKPETEDPDSPKDVYTSTHYIGIELHDEHYSWSLPDDVFEAGETKPTRPLKKKAAQPPNGAKRKTPTEGALNSAIDAAKRQKTSTVSAG

SEQ ID NO: 125 - protein, 588 aa, Wallemia mellicola
MAEKQWGITPPISLASPTDLEKQVDAGLIQELKDQNTIESEQESLNREKVLALVQSLVKEFVKRVSIRNGMSESLADQAGGKIFTFGSYRLGVNQPGADIDTLCVVPKHVSREDFFDVFEPLLKTREEVSVCAGVPDAYVPIIKTTISGIEIDFLVARLALATIPDDLELADDNLLKNLDERCVRSLNGSRVTDEILRVVPNVQVFRESLRCIKLWAQRRAIYSNVNGFLGGVAWAMLVARICQLYPNENAGAVIAKFFTILYQWKWPHPVLLKEIEDGPLKVRVWNPKVGSSTLLIYPSDRAHRMPIITPAYPSMCSTHNVGPSTQEVMRQEFKRGMDILDGIHKGTTTWSQLFNKHEFFSKYKHYLQIIASGQTAESQKKWSGAVESKVRQLVSKLELVDGIELAHPFVKGFSEVFICLNDEEVQHAAEYNPSEEVKERSKKPEEYENKEAEDGIHRVYISTFYIGLAIQPRNPETNEKRTLNLTYPTNEFMKLTKLWDQFVEGEMKIFVKNIKASALPDVVFEGGIRTSSSKSSKKKSKRPSNVMDESNGENEQNKRLKPDNTSNNEQKSEATALAPSAVPTEST

SEQ ID NO: 126 - protein, 587 aa, Xylaria flabelliformis
MAEKTWGITKPISSAPPTPAESHATALLLEELRRQNTFETASEIKKREKVVNDLQRIADEFVRKVARAKEPHNEVLIRDARGEVFTYGSYCLGVYGPGSDIDTLVTAPRYVTRDDYFKYFPDLLTEMAPPGAITNLTAVEDAFVPIIKFEYWGISIDLIFSRIATLTQFPPHKQLELTSNEHLRGLDDRELRSLNGTRVTKEILNLVPEQSTFRTALRAIKLWAQRRAIYANIIGFPGGVVWAMMVARVCQLYPKATSATIVGKFFLVMKGWPWPIPVQLKHMEDGPLNVRVWNPKIYKSDSFHLMPVITPAYPQMCATFNITKSTKTIIQRELERGVELTDKILGSERPWKDLFVKHTFFTQDHKYYLAVIATSTTKEAHKIWSGFVESKVRILVGELERHSSIALARPFNKGFERQHLTKTDQQAAEVQSGSLAYVTEDEDNSDDATVTKAENGTDGNADSSDQKVTAENTTQPRRTYTTTHYIGLELAEGSKSLDLSYQVNAFKQLCSEWEKYSAEFNSLSVQHVKNINLPEDVFEPGETKPARPLKKPTANGTAPTTQRKRNAPTEGHPPPAKRQQSSAAAAG

SEQ ID NO: 127 - protein, 592 aa, Chaetomium globosum
MSEIIYGVTPPISTTLPTEPEKRLNHALHQELRAQGTFESPQETEKRKEVLRQLEKITTVFVQRAAAQKEPKNTFMIRDAIGRVFTYGSYRLGVYGPGSDMDTLVVAPKYVTVEQYFEIFPEVLVEMAPPGAITDLTPVPEAFVPIIKFEFSGISIDLIFCSIQTLKQLPDEKNWSLADNNLLRGLSENEVRSLNGTRVTDDILHLVPEPATFKLALRAIKLWAQRKAIYANIMGYPGGVAWAMLVARVCQLYPKATSAVVVNKFFHIMFKWPWPLPVLLKDIEYGSPVTRVPVWNPKSSRRAGPKRHRTSMRASSNRGCGCWLTDLRGTILLPWPGPSTRVTTENTDARTIGSWEEVVSVGSLAYVYKPPADSEEKAKAEPKVEPKREVKQEIKAENREPAAAEEKLETRGEDGMRIKQEHSESTQPPPPSNVKPEPHHDTNSEVKLEDIPQKKKDEPDEMEIYTTNHYIGLQLVEGAKSLDLSREVNDWKSMCTSNELFDEGLMFLSIQHLKNTNLPDDVFEPGETKPRPVKKNLKRAASEGPGNMQPQPPVKRHAPGKPSSHSAQQQQQQQQKQRQQRPASTAAAAVAG

SEQ ID NO: 128 - protein, 591 aa, Lachancea thermotolerans
MNQQKTYGVTGPISTAGPTAAENQLNDALIQELKKEKSFESEEDTKKRVEVLRILQNLAQEFVYQVSKKRNMSDGMAKDAGGKIFTYGSYRLGVHGPGSDIDTLVVVPKHVTREDFFTVFDQILRTRSELEEIAPVPDAFVPIIKIKFSGISIDLICARLDIAQVPVNLTLADKNLLRNLDEKDLRALNGTRVTDEILQLVPKPTSFKIALRAIKLWAQRRAVYANIFGFPGGVAWAMLVARICQLYPNACSAVILTRFFHILTKWNWPQPVLLKPIEDGPLQVRVWNPRIYAQDRSHKMPVITPAYPSMCATHNISESTKKVILAELERGAQISSEIFSNKKTWSDLFQKHDFFYKYKFYLTVMASTSGSSEQHLKWSGLVESKLRLLVQKLETLGGINLAHPFTKPFEASYVYDNEAQCKDIIDNYGTHKAQDILAQYTEVTDDNKDTDGVKDKAQVHITTMYIGLDVALDGKKQVDIHVPCSDFFNLCRSFSEYDDTDMFSLMIKYVKLYDLPDNVYVEGEERPVKHSKRKKLGKDSKKSKRPKSNSLKAEEPTSGTTNSEKNNSQGPNKPVKHEPPMQTSPNPLAGS

SEQ ID NO: 129 - protein, 558 aa, Schizosaccharomyces octosporus
MSVKQWGITPPISTAPATEQENALNTALIDELKRQNLFESSAESERRIKVLDDLQRIATEFVKKVSLAKHMNEKMANEAGGKIFTYGSYRLGVYGPGSDIDTLLVVPKHVSRENFFQDLEPMLREREEITELASVPDAYVPIIKFKYFDISIDLIFARLSVPKVPRTLELSDNNLLKGVEERCILSLNGTRVTDQILQLVPNRAVFKHALRAVKFWAQRRAIYANVIGFPGGVAWAMMVARICQLYPNAVSSVIVAKFFRILHQWNWPQPILLKPIEDGPLQVRIWNPKLYPSDKAHRMPIITPAYPSMCATHNITPSTQAIILSEMVRAGEIADQIMVGSVGWSALFQKHDFFHRYKHYLMITAASKTAEGQLKWSGLVESKLRHLVTRLELVEAIALAHPFNKGFDKIHNCKSEEEAHQLASGISLDVASQTADLERLAADANGQNENEDEKEEKKIYPVYTTTFYIGLELEKKKGQPIKKLDISWPAQEFYELCKKWDRYDETQVNVFIKNTRNVNLPDEVFEPGEEKPKSSKKRSYNDAGNNNETMKRQKISTA

SEQ ID NO: 130 - protein, 608 aa, Exophiala spinifera
MAEKQWGMTPPMSTALPEPIDTEKTADLIEELKKENNYEPLEATQKRMATLGLLNRVTQEFVREVSRRRRMPPSQIEQFGGKIFPYGSYRLGVFGPGSDIDTLAVAPRHVTREDFFEYFPTVLKRMTAEGDISSLTPVPDSYVPIIKLVLNDIEIDLIFASIASLQTIPKNLTLNDNNLLTGLDQATIRAVTGPRVTDEILSLVPEQKTFRTALRAIKLWAQRRAVYANIVGYPGGVAWAMLVARVCQLYPHAVGATLVDKFFFVMKNWDWPTPVMLKDIEQPKPSQANEFKVWNPALYKGDKKMLMPIITPAFPSMSATYNISKSGKTVILRELERASQITNNIFAGKARWSDLFKKHSFFTADHKYYLGVTASTLNADSAKQWAGLVESKVRIFVMLLEGIPDITLARPFTKGFKRVHKCADETQIREVQKGSMKYKFEETKTVETTDPELVTSNGDGAAVPAANGAKPETDQGAHTVYTYTFYIGIDTTAKGSLNIAPSFQNFKDICEGWASFNRDSHFLTLASVKCWDLPDDVFDAKAGEVKPSRPVKKVAKQVKAEAKSGVRRSINEVEATETNGDAAKRQKLMTPTPTPTPTPAPKPTAAPA

SEQ ID NO: 131 - protein, 626 aa, Scedosporium apiospermum
MADRQLGVTPPISMALPTPAEIEANNAMVEELRKQGIFESKEETDKRNLVLESLQKMCDEFVTRVAQEKDQGLARDARGKIFTYGGFRLGVFGPGSDIDTLVVAPKYVTRDDYFAIFPDLLLEMAPKGAITGLAVVTDAFVPIIKFEYWGISIDMIFSRIAMLKKLPTNTSQFNLTDTALLRGLDETEIRSLNGTRVAHEILDLVPEQSTFQMALRAIKLWAQRRAIYANVMGYPGGVAWAMLVARVCQLYPRATAATIVSKFFCIMRQWPWPQPVLLKHIERAPLGYRIWNPVVYPSDKHHLMPIITPAYPSMNAAYNINRSSMSIIQTELSRADGITDGIVVGQRPWSDLFEKHTFFTADYKYYLAIVSSGITKDAHKKWSGFVESKVRMLVLALDRHDTIALAQAFVKGYDRVHKCNADSEIQKVQQGDLAYMIKEEDAPKEEQTSPVKSETKPDPGANEKNSAGGSNSNGDVAGSADAVKNEEGANGVKAGDENVYIYTTTHYIGLALRPGQKSINLANEVGEFKKLCKGWEKFDHQLNAITVQHLRNSDLPDDVFAEGEVKPRKRVVRKANGTGPSPPVDAKQATNGTTSQVDSKKRAAAETGAAAPAAKRRQPASVVAAG

SEQ ID NO: 132 - protein, 613 aa, Trichoderma citrinoviride
MAEQNYGVTPPISVTLPTEAENRASDALLEELRRQKTFESPSDTEKRHEICNEFVRKVAREREPKNEILIKNARGKVFTYGSFRLGVFGPGSDIDTLIVAPKYVTREDYFNYFPDLLVSMAPPGAITDLTVVKDAFVPIIKFEYSGISIDLIFSRIIQKQIAPDFHSLKDSSLLRGLDEAELRSLNGTRVTDEILELVPEKSTFKLALRAIKLWAQRRAVYANIMGFPGGVAWAMLVARVCQLYPKATMSVIVNKFFLVIGQWRWPQPVLLKPIEGGPLPVRVWNPKVYKGDSFHLMPVITPAYPSMCATFNITRSSMTIIQRELRRGLEISEQIMVGKRPWSDLFVKHTFFTSGYRYYISVVSASKDKEAHKVWSGYVESKIRMLVQKLEQHPSIALAHAFNKGYDRRHLCRNEQEIGQVQEGSLEFMIKDWDDNKLNGAAIKPEKVEGDSKPNGADIIQPEKVEGESKLSDIPVKPEMDEAESKLSEVAIKTETKDGSTESVFPVEVYTTTHYIGLELEEGAKSLDLSYQVDEFKVLCTSWKKYQEDLEHLVSLGVQHVRNFNLPDDVFEPGEQKPQKKSSAKSLANKKRGAPEDNTPPAKRQQASVAAAG

SEQ ID NO: 133 - protein, 598 aa, Aspergillus thermomutatus
MSAPPVRQWGVTPPISTALPTPDELAANDDLISELKAQNNFESPAETERRKQVLQLIQRVTHEFVKVVSRKKGLSPAAVEAAGGKIFTYGSYRLGVYGPGSDIDTLVVGPKHVLIDDFFSDFPPVLEKMAPPGAIEKMTPVPDAFVPIIKLELSGISIDLIFARLIVSSVPLNLDLKNNDYLRGLDEKEVRSLNGTRVTDEILELVPQQKTFRLALRAIKLWAQRRAIYSNIVGFPGGVAWAMLVARVCQLYPHATGSVIVGKFFRIMNKWAWPQPVLLKPIEDGPLQIKVWNPKIYHGDRFHLMPIITPAYPSMCATHNISMSTKAVILRELQRGGDIVDKIFLKQLTWNDLFARHSFFTHDYKYYLSITASSRTKEAESVWSGLVESKIRHLVGALDRKPTIAVAHPFPKGFERVHIISNEEEAEAVKNGSTKYQDKGTKTETTDETKDAAHQAAAQSGVENAEVEPVGENANGDSRIIYTTTYYIGLELKPLEPGASRSLDISTDAQIFKSTCTSWAGYQPGINDLSITHVRNFDLPEDVFQPGEARPTRPKKKVIKKPEAGAQKRGIDSLDDASLPAAKRQVTSNGISSTPTPA

SEQ ID NO: 134 - protein, 579 aa, Sodiomyces alkalinus
MQDRPLGVTPPISTSLPTDAEHAVNESLLEELKRQNTFESPAETAKREKVLEQLQLICNEFVRRVAREREAGSEALIKEARGRIFTYGSFRLGVYGPGSDIDALVVAPKYVTRDDYFKLFPGLLQEIPPTGAITDLAVVSDAFVPIIKFDFLGISIDLIFSRIASLKQLPKDKDWNLKDSNILRGLDEAELRSLNGTRVTDEIISLVPEPSTFRLALRAIKLWAQRRAVYANIMGFPGGVAWAMLVARVCQLYPKATSSVVVNKFFHIMRRWPWPQPVLLKAVESGPLQVRVWNPKLYKGDQFHLMPIITPAYPSMCATYNITKSAMTVIQRELQRGCEITDSVMMSKQPWSDLFVKHAFFTSDYKHYISVITTSTTKEAHKIWSGYVESKVRVLVQGLEQHPSIALAHAFNKGYDRRHKCSSEQEINQVQEGSLDYLLKAGDSSAGDPAEGNKSPRRPADGEHPEITPEATETIVFTTTHYIGLELVEGAKSLDLSYQVDSFKQLCGQWEKFNSNLNYLSVQHVRNVKLPDDVFEPCETRPQKKSATNGSLQKKKRGASEVDMHPPAKRQQSSVTAAG

SEQ ID NO: 135 - protein, 606 aa, Neohortaea acidophila
MDPKLGVSGTMSMEPPSPKDIKLDDALLQELKSRNEFEAPEETQRRHSVLDRLESVLKRLVAMVGKQQGLPPGILNEAGGKIFTFGSFELGVYGPKSDMDTLMAAPKHVSREDFFQYMPDLLRKEFKPEEIAELTPVPGISVPIIKLELCGVSVDLIFCRLHLQSVPKSQELSNLDLLRGLDDTDLKCVNGTRVTRRILELVPQTKVFRMALRAVKLWAKQRALYGNIVGYPGGVAYAMMVARICQLYPRAAAPLVIWKFFYLMRKWNWPSPVLLQNHEEGSINLREWDPSIYPGDKRHLMPIITPAFPRMNACHTIGPSTKKVLLQEMERAEGIVRSIYESGRPWRDLFQRHSFFTDAYRHYICVITAGRTKEAQQAWSGLVESKVKWLIVGIEHSDAKSVELVQPYNKGFNRVHECKGDADIDKTLDGNLDCQVKEIKTVTTEQAGDVQFQAAAQTDTDGQEVPAPNGEAEVPPQTDGPQTIWTTTFYLGIGLTKGANSLDISSPIRDFTQQCKEWQNYDENLHSIRVKHMRNYDLPADVFAEGETRPTRTKKKSAPKTTDPTAMDAANKKRSFNNSGLDTLDDPAKRRASANGTATPNGVPPR

SEQ ID NO: 136 - protein, 405 aa, Schizosaccharomyces pombe
MNISSAQFIPGVHTVEEIEAEIHKNLHISKSCSYQKVPNSHKEFTKFCYEVYNEIKISDKEFKEKRAALDTLRLCLKRISPDAELVAFGSLESGLALKNSDMDLCVLMDSRVQSDTIALQFYEELIAEGFEGKFLQRARIPIIKLTSDTKNGFGASFQCDIGFNNRLAIHNTLLLSSYTKLDARLKPMVLLVKHWAKRKQINSPYFGTLSSYGYVLMVLYYLIHVIKPPVFPNLLLSPLKQEKIVDGFDVGFDDKLEDIPPSQNYSSLGSLLHGFFRFYAYKFEPREKVVTFRRPDGYLTKQEKGWTSATEHTGSADQIIKDRYILAIEDPFEISHNVGRTVSSSGLYRIRGEFMAASRLLNSRSYPIPYDSLFEEAPIPPRRQKKTDEQSNKKLLNETDGDNSE

SEQ ID NO: 137 - protein, 333 aa, Trypanosoma brucei gambiense
MPPSPAVVGRSLVNSFKQFVSKDLHTRHVDATYRLVLDCVAAVDPLMRLYTFGSTVVYGVHEKGSDVDFVVLNKTDVEDGKGGDAATQVAKGLQADILAKLARVIRQKHLSWNVEEVRRTRVPVVRVKGGGAVDFDITAYRRNGVRNSALLRAYFEQNPPCRWLSMSIKRWSKQTGLNASVIGGSITSYGFNLMVVYYLLQRNHLQFVPPSTIDVSRVEPLPPHLPLEEPADEGLELGTQVLDFLHFFLHEFDSDKQVISLNRPGITTKEELDWTKSAEDFARMNGEKVHYQWCIEDPYELNLNVGRNVTPLKRDFLRRHLEKARDTALLTIV

SEQ ID NO: 138 - protein, 349 aa, Schizosaccharomyces pombe
GSHMSYQKVPNSHKEFTKFCYEVYNEIKISDKEFKEKRAALDTLRLCLKRISPDAELVAFGSLESGLALKNSDMDLCVLMDSRVQSDTIALQFYEELIAEGFEGKFLQRARIPIIKLTSDTKNGFGASFQCDIGFNNRLAIHNTLLLSSYTKLDARLKPMVLLVKHWAKRKQINSPYFGTLSSYGYVLMVLYYLIHVIKPPVFPNLLLSPLKQEKIVDGFDVGFDDKLEDIPPSQNYSSLGSLLHGFFRFYAYKFEPREKVVTFRRPDGYLTKQEKGWTSATEHTGSADQIIKDRYILAIEDPFEISHNVGRTVSSSGLYRIRGEFMAASRLLNSRSYPIPYDSLFEEA

SEQ ID NO: 139 - protein, 480 aa, Terfezia boudieri
MSGPPSGPPSGPRYRGGHRNFGPRHLQSQERQQQQQQQQQQQQQPYQPSPQELAVLAEVVIEQIALATPSAEELERKTKLRERLRKLCTNVSPTAELEAFGSLVSGFATVGSDLDLVLVDKGGSTNRPFFEVPLLLEQELKDNGLEAQLLAKTRVPILKIKQPATEEYPKEVAADIGFANPLAIRNTDMLSMYSKCDPRVIDMVRFIKRWAKRRKINNPYKGTLSSYGYVLMVLHYLINIVEPPVLPNLQLYPIPAETPKDEITTEEGHNVWYYKDVAEIQRRVAEGTMTTNTMDLPLLLLGFFEFYAYRFGWVKDIISIRTKGGLISKVDKGWTVVAVRVGKCEAEYKDRYLFAIEDPFETNHNVSRTCNIYGVRKIRDEFKRANRIMRMREGVGALRYKLFEEAPEDVRPQHHQKQSIAGEEHVNGAPESGREDREGNQLVEELEGHSNGNGIHPSEGYPGPGLAAVVEGVQRLSVDA

SEQ ID NO: 140 - protein, 761 aa, Drechslerella stenobrocha
MELTTGDFGGPPGASNATGPGQKPEDPRTMDPVGEQTLESRLRGMLLHGGPPPVDPRTQLRHPSGLAQQAQQAQQQPIGIDPAVPGHLHQLQTAAAPPQQSRLPPSPNIHHQHLQPRLLQRSNTLPPPGGLTQPPPGLERAGTFHGPVPVAQFGYHGSYPTAHSQRGRYGNRRNGFQNAPVPQMNNNLHFPPLGTVPSQPPPPLSERSSLSNMNNPRRHSGPGHYHTDNNHSPPYKRYEAFHGAPGSPQGYSGSPPQNRQGRPYHSSRGSHGGRNNRNFWPLDYDGLTNYAKYIVESVSPTPEEIAMKDNMLRRISEICDKLVPGSRIIPFGSLVSGFATKGADMDVIFAHDTIDPAPSSHESNIPVRLANEFLKRGFEVDLLIKTRVPILKLKTPGEPVSRPGSPVSEGDGDSSEEPWPENVSCDIGFKAHLGITNSHFFRTYSHCDHRFREMVLFVKQWSKNRDLNSPYFGTLSSYGYVLMVAHFLINVVQPPVLPNLQLIPPAAETAESELMQEGFNIWYFKDLERIASGEFLPGGRNEMSLGQLVHEFFQYYTTNFNFVSECVTIRTPGGVMYKQEKGWTSARERVGEMNNTYQDRYLLALEDPFEVSHNVGRTCGGAGVRRIRAEMQRAAHIIRKVSAKEGGTPRSAGWEMPLVVEDLMTTVRETNRGFRNRRVNQKNLMEWIRNDWTVGKCLVQIDAAAEELRKKRELGLPDDAELPPESEEDSEGDEDASEEEEESSSDDGAVRVVGGGSRGGV

SEQ ID NO: 141 - protein, 337 aa, Phytomonas sp.
MTSLSAVNVGKLVCDSFGKLLSKNLDIKPIVQTHEIVSQSMQTVDPSMALYVFGSTAVYGFHESGCDVDFVALNQKDVSDGKAADPSSEIAKGLQVDFLSRLEASLREMHNLAWKMDLVRRTRVPVLRVKGDPCGIDFDVTARRRNGVRNSALLSAYFKQKPETRWLSMAIKQWSKRAGFNMSVDGGCLTSYGCNLMVVYYLLQRQLVKFVDPERCDVAHIPSLPSYLPLEHPAQNGSELGDMVLDFLNYYLHEFNPETEVISLSRADKTTKEMIYWTKQAEDMARISGEKVSYRWCIEDPFEHNLNVGRYVTPFKLTLLRKHMERAKETALLLSIS

SEQ ID NO: 142 - protein, 355 aa, Bodo saltans
MLSPAAIGKAILDHHASLIGADLSVKRVHDAHSLVKNSLDSVAPDLKLYTFGSSTVFGFHEPKSDVDFVALRQEDIVDGKGGDSTSQLAKGLQTQVLAKLAASVRQKNVQWAVEEVRRARVPVVKVKAPHIDFDITAHRRNGVRNSALLRHYLTQVPENRWLSIAIKSWSKRVGMNGPVGGYLTSYGFNILVVYYLLHRRRHEQPEVEQKEGQDGAASASVDGKQQKELTFIDKDTLDVSLIPPIPEYLALEPPNPETLGEQVLDFFDFYLSRFPMESHVISLSHKEPITKQSLNWTKTAEDMKNNTSMEKVFYRLCIEDPYEVNLNVGRNVSPFKFDLMKKHFVKGRATALGLL

SEQ ID NO: 143 - protein, 425 aa, Angomonas deanei
MPPNPRQLGAAMIEQFRPLLQAPQQSQLTVLSEAPLFQRIQREVHELDPGARVYIFGSTRVYGFHDSGAYQQQSAQVPPSDVDMAVLRREDLLDGSGVDPSSELTRNVQAEFLEKLLNRLQQSEGSASIRSNCSSLVKRTRVPVLRVKHLVSNHSPPHDKEDNISVQVPFDFDITCGRRCGIRNSALFFHYVQQMPMIRFLLLSVKKWSKQTGLNSAIKAPYSNNNNQNHNLTSVMGGSLTSYGFHVLLLYYLLRRGVVQYIPMTVSSAAGSTDRIPNPQLDVNAIPPVPTFLPLLDPALEPHGPQGDPVRYLGELALDWCHFYLHEFDVEKEVVSLSRFDDYFADSPPSGPFPVITKDVLGWTKQREDQGRLRGEKVHYTLCIEDPYEVNLNVGRNVTSLKWMLFRKHLEKAIQTGLLLCPPSK

SEQ ID NO: 144 - protein, 432 aa, Protomyces lactucae-debilis
MPSSSPLGATLPNSSSLAVSAAAAVVPDPLFAFTQFAVDAVRNNTVSHAELVEKERLRQELEAVLQALQPTARLVVYGSLANNLAIANCDMDLMDALHDVSVDLPALYTEALNSQGYSVKLLSKTRTPIVKVSLEHMAVPYCVDISFDNALALHNTKLIATYAACDSRVPILLMFVKLWTAVRRINDPHSGTLSSYGYALLLIFYLQNRCNSPPVLPNLQLISAMGTRSADELECSGYDIWFFKDVEKIQQAQVVKNTSSIGTLLEGFFSYFAYEFDFRDLCISIRTPGGIVTKTAKTWTQMVEHLNDRGDAKVKDRYILSIEDPFEIVHNVGRSVTRAGLYEIRGEFMAATRYVRGNKLDEILNPHRREIVAPTSSTAMHSPQQQHGTQQVSPGEGSSPTPPAVHAEARPSTGTATSTKTSQQAETVVKAE

SEQ ID NO: 145 - protein, 437 aa, Strigomonas culicis
MPPPTPMQLGRAILRHYAPSIVVPTAATSGPISSAAASTASLASIHPLFEFIQGCARRVDPQTHLYVFGSTRVYGFSEVSAPTSVGPPAALARPVKNDVDIAALSAADVATTPSPHSADAVVDKGSELAKSLQIDFLEKLKMQLQTQRQHQGDGAPPAASLSWEMEVVKRARVPVLRLQPRPAYTTSAAGGVPTYNVDVTYGRRCGVLNSALLRRYMDQQPDLRYLCLAVKRWSKLTGLNTATSPDGGGLTSYGFHLLLVYYALRRRLVAYVAPENIQWGDIAPVPAALPLRFPGDADGADAWRGRSLGERIAQDDAAAARVGEWALDFVRFYLHEFDYERDVASLSRGAAAAGLVTTEALQWTRQEEYAARGRGEYLFYRFCIEDPYEINLNVGRHMSSVKLMIFKKHLEKALETGLAFIPADEKAKQRVKPKGKE

SEQ ID NO: 146 - protein, 467 aa, Basidiobolus meristosporus
MSLETDRIVSEFKDLAISQTSETNQPVPDVKVSSVEDSQHHSKAHPPKNENGNNQGHRHGKKPFHRGSTFGEQGKSDRSYGARSFHPNRGTGQYEGKRVLDKSDPRYLKARARFISVLTEQVSELYLKLTPSVEEVSRREQLYFRIHSICEELFPDAQLFQFGSTANGFGLKNADVDFCLCTAENSSLTAVAFVEQLGSVLKERGMENVMLLTRTRIPIIKLKDPVSELNCDIGFNNRLAVYNTRLLRMYAEVDSRVKEIVAIVKHWAKSRQINEPYLGTLSSYAYVLMIIFVLQQRGVLPCLQQICEGEKKEVLVDNYDAYFFDNITDLPKYWKSQNNESLGELLYEFFRFYASDFSYYGSVVSVRTGQVMSKEEKGWTAEATRESGASHVQDRYWVCIEDPFEVTHNLGRPVNKNSLYTIRGEFMRAANILGGTRTSTILERLCQVYVPEEEPPRAPPAPHERSN

SEQ ID NO: 147 - protein, 354 aa, Neocallimastix californiae
MKTTERTCTFEFYDELTEEINKIVNKIKAKPNDLKKRENLFNYIKLVAKKIFPNSKAYKYGSIENGFSLTNGDIDICILNNKEFNNTPAECVEILGEKLKEDGINDIKLLIRARVPIIKMKDNKSGISCDIGFQNKLAIQNTKLINAYSKIDDRFKQMVFIIKYWSKMKNINEPYMGTLSSYCFILMIIHFLQIKEPPVLPNLQKIYLDNFIEYEYIDDFNVSFFENIDELHKYWNSKNNESLGELLVEFFKYYANDFPYITGVASIRVGNIITKEEKKWTREHQFEINKSNSVKDRYWFCVEDPFEITHNLGRPVDRKSLFRIRGEFLKAVKIINNKNLTALQVLEKLLEKNI

SEQ ID NO: 148 - protein, 344 aa, Perkinsela sp.
MLDPSETCMGRIKNIHAIITSKLMGLLVEKNAQIYCFGSSMVNGVVEKKSDVDVVYLTREDLQKPLSIGDVCNPQSRSEQTSILSAISKILMKDSELFSTVQEKPRARVPYVRGVLKNGLEIDISAHRRNGVRNSLLLRSYFSQIPPTRPSLPNATTSVYRMLSLALKFWGKRTGLVDPVQTFLTSYAFNVLICYYLQQRGGMDFIHPESILIPKGHPTVPDYREIALAGTQGHTCLGWYMRDFLKFYNHEFDYNNTVVSLSRKGITTKEYLGWGLRDEERMHGTDGNAFFYRFCVEDPYENRLNLGRFVTPLRYSMFRMALHQAQLNGFGYLNLKNYGAKIVD

SEQ ID NO: 149 - protein, 352 aa, Saitoella complicata
MMAHPSQGGAHPLSAGPTNPQPYQDAYSGFTEYVLSINRPQYPSANEMYLKEQLRSLIDVEIVQRIQPSARVIPFGSLVNGFATANSDLDLCILDDSPNPRYKIKTELPEVLAAEFERYGFEVKLLTKTRIPIIKLVQGPTAQFPLGLAMDIGFENRLALHNTRLLATYSRIDSRLREMVLFVKHWAKVRGINSSYHGTLSSYGYVLTILHFLINVASPSCLPNLQHIGAQVPVPFEELECEGYNIWFFKDLTNIPPSLNRRSIGELMAEYFSYYAQFDFRNMVISIRTPGGLLTKTSKGWTQALDRVGPEDQKVRSRNFLCIEDPFEITHNVGRTVGKNGLYDVWPFVSFA

SEQ ID NO: 150 - protein, 388 aa, Steccherinum ochraceum
MATVFAQSHSLLNTRQPHQHVQQPAQQPPQQQQQQQSTRPKQLSKPRFYAEFSQCLFDFVIQLLPTPEELAIKEDVRKLLERLIRTLEPDSRLLSFGSTANGFSLRNSDMDLCCLIDSDDRLAASDLVTMLGDLLERETKFHVKPLPYARIPIVKLSLDPSPGLPFGIACDIGFENRLALENTRLLMCYAMIDPARVRTMVLFLKVWCKRRKINSPYKGTLSSYGYVLLVIFFLVHVKNPPVLPNLQSMPPLRPMDKEESTLNGHNVWFFDDINLLRQRWQSTNTESVAELLVDFFKFYSREFPYNSGVVSIRAGGIKKDSKGWFSEAERGSARERNRLCIEDPFETDFNVARCVTRDGLYTIRGELMRASRILAARPERPIVALAQL

SEQ ID NO: 151 - protein, 421 aa, Gymnopus androsaceus
MAAYSPTMSSNQHYHELNSASPPARRKWLADFSECLFSFVIQLLPTQEELAVKEDVRKLLERLIRTIEPESRLLSFGSTANGYGLRNSDMDLCCLIDSEERLAASDLVTMLGDLLERETKFHVKPLPHARIPIVKLSLDPSPGLPLGIACDIGFENRLALENTRLLMCYAMIDPTRVRTMVLFLKVWSKRRKINSPYKGTLSSYGYVLLVIYFLVHVKNPPVLPNLQQMPPLRPIPKEETYLGGFNTWFFDDIELLRQRWHSENTETVAELLIDFFRYFSRDFPYGVGVASIRAGLLKKDSKGWQNDLSASRYNDARERNRFCIEDPFETDYNVARCVTKDGLYTIRGEFMRASRVLAARPERAILALADLCDERKDEDPCRRASLYSFSSSAPTNTVFGGKSINAFQRNTDYSLSFDQTP

SEQ ID NO: 152 - protein, 512 aa, Trypanosoma equiperdum
MGVRLYSCDACPHAVFTTHAALLAHAEEHHADLLPDHARLRRIAQKLNPVWNRALNARRNTITSWGKKIFHVAAQRDAGESKMQEAHRARAQLECVVRRWHDKARVFIFGSSVAMGVWDGTADIDFAVVDVDAMERGSWPPLEKNAVRSITELLRRVGFSFVNLEPISHARVPIIKHHASSPILTVARRDAEDVVARSIRFILNGPATREDRLLLEGSVRDAVGPTGVQQVWWNRTSDMMSATLESTTAAVRAAMCSPALASASLRTKVQPAHDECRPELYNIDFALSFRAFGIRNSTLLRKYLLSHPCARPGAIVLKDWSKTSGVNNSVNGYFTSYAINIMWIYYLVQKGYVPYVDPLEIPESLVNYTDFDPRYTPMIDPEITNTEREELYKAAGDMLVGFFYFYSFEFDWGHNVISLNRPGITTKRMLGWHVEDVVPVASTSVSSGGGGSNVKRHPTRYELCIEDPYEENLNLGRHIGVTKSLRVRTELYRGLLSLLKEGETRSCVFAAA

SEQ ID NO: 153 - protein, 531 aa, Morchella conica
MTAGRDIRSPPRGQRGQGFNKSQNSPRALPLNALDHFPPLGTQPTKAKTPPPNYSQQQQSTTAKPQQRTQYQPLPIDTRNTNPHHARGEVHGRRNFRGDYNQRQLRRDIGPEVCQYLSELSKKVIAEAAPPESEILVKRALLERLEAISKTIVPDAKLIAFGSLVTGFATANSDLDVIFTGGDRVDILNESSDDPSSNFRIPMLLESKLQEEGFGTTLLTKTRVPILKLVQKATEQSPYELQCDIGFSNHLALYNTQMLLTYSKCDPRVKEMMIFIKWWAKRRHINNPYRGTLSSYGYALIVLHYLINVVKPPVLPNLQTFPVPDSAPTNEIIFEGDSDDTFEIWFYKDIEKLPKSDNAMDIGELLKGFFEYYAHNFQWGREVVSIRTKGGLMTKQEKGWVAAVIKPGRTENSEVKNRYLFAVEDPFETEHNVSRTCNGPGVNRIKDEFKRAVWLIRVRDGGKTLFQNLCMEAPPERVWVRREDRDRRGIEEAGGHGSKDHQGAEGEAGDGVKHESVVLSEDGRNALESLV

SEQ ID NO: 154 - protein, 474 aa, Pneumocystis murina
MDSVKGFEGVESVVFQKEMKKLTKISDFSGKNYGSGNEIEDLKKSLEKMMLESVNIQEKGVFLREKGSEGSYEANLNDKDVLKNNKKDSNFRNIKYYEPKDYGLYVNGKAVWRKNYDEYFVLSTFISHTLKHIMPTNEEISQKERFRFFLSEILNTCRPSAKLVLFGSVASGLAIVNSDMDFCVMDDSLDLHTDEFLKIFSEEIKKYGMETTLLFRTRVSIIKVNSKGSFNFPQGISCDIGFNNKLAIYNTKLLATYSKCDHRVRKIILFVKYWAKRRKINDPYHGTLSSYGYVLLILHYLINIVPIPLLPNLQQMKVASWRSIPQSEIECDGYNVWFYKDIDISCSFSNNTDSLGKLVYGFFYYYAYQFNWKDHVVSIRTQTGLLTKQEKGWTQAMERVFVSDKIFKDRYILAIEDPFEITHNVGRTVNKQSSHIIRGEFFRASKLSSSKLRKKIFNELCEERIIINNFSNVI

SEQ ID NO: 155 - protein, 445 aa, Schizosaccharomyces japonicus
MSGNGNGHFIPGVHTVEEIERQFVNLALQKRNQAAAAAAAAAERELDPVTCFLLSTYDDVRVSDDELREKDAIMNLLKHVVHSVRPEADIVAFGSIQSGLALKNSDIDACILLPDIGEEMEEFASECFERFTALGFEGKYLRKARIPIIKLLSDTKNRYYYGFQCDIGFNNQLAIYNTSLLHQYSLIDPRCKQLAILVKYWAKQKRINSPYYGTLSSYGYVLMVLFYLIHVVRPAVLPNLQDSPHKQDLYVEGFNVGFVRGTTVARRNTESLPQLLAGFYGFFAHEFNYRESVISIRQPGGLLKKVDKDWTLAKEHTGSADQVIKDRYVLAIEDPFEITHNVGRTVSKAGLFEIRGEFMQATRLLNAATLHSTSAIRKLRASLFKERLEPKSHLKYQKALRQKRMGTEGKAAAEGKTNNPTAASGSDSAPSESASRTATVESREP

SEQ ID NO: 156 - protein, 620 aa, Ascodesmis nigricans
MNPPQQTTGLEDHLRSLIINNQTPPNHSISPAAKIPHKHHPRTSTDSRGFVRSQTIHRPGPAASQPPSNGPNRRFPGEAQRPDHRPPHLRGPSQNRNYWHGAPPPPRQVVPQGLSPPAATNLHHFPPLGAKVAPPPPSLPSYHSVPTAPCHRPENQRPVYQNDNRDSYHYKQRGLYDGYLAEQWRELDKMAKEVLLTATPVDGEKEAKEKFMQDLEAVCQKVEPTAKLIPFGSIVTGFATRGSDIDCVFTSNTDSALQNDKITEPETPFDRHDELVTQLQESGYNAQLLTRTRVPIIKLVRPATPDVPDSVSCDIGFNNFLAIHNTRLLRTYAACDERLVQMVLFIKWWAKRRHINSPYRGTLSSYGYALLIVHYLINIAQPPVLPNLQLFHPPATQTSTELQYEHNGNVCNIWYLKDTSTLPRSANKASIGELLRGFFEYYAFTFRWQNDVVSIRTAGGILTKMEKNWCAARSRPGGREEGQVWEVKDRVDVQHRYLLAIEDPFETTHNVARTCTFNGVGRIKSELKRAMYLIKSRDPGHHRLRTELFEEAPPERPFVRNGKEVVDKLEKVEIVEERAGVAVEIVEERAGVAVEIVEERAGVALEVVEERAGAAVEVDV

SEQ ID NO: 157 - DNA, 18 bp, Artificial Sequence
  primer
  misc_feature (17)..(17): n is an inosine
ccccccccccccccctnt

SEQ ID NO: 158 - DNA, 12 bp, Artificial Sequence
  primer
  misc_feature (11)..(11): n is an inosine
ttttttttttnt

SEQ ID NO: 159 - DNA, 12 bp, Artificial Sequence
  premier
ccctccctccct

SEQ ID NO: 160 - DNA, 15 bp, Artificial Sequence
  primer
ccctcccctccccct

(19) United States
(12) Patent Application Publication
(10) Pub. No.: US 2023/0313255 A1
(43) Pub. Date: Oct. 5, 2023

(54) Massively Parallel Enzymatic Synthesis of Polynucleotides

(71) Applicant: DNA Script, Le Kremlin-Bicêtre (FR)

(72) Inventors: Adrian Horgan, Le Kremlin-Bicêtre (FR); Henri Lachaize, Le Kremlin-Bicêtre (FR); Domiano Verardo, Le Kremlin-Bicêtre (FR); Xavier Godron, Le Kremlin-Bicêtre (FR)

(21) Appl. No.: 18/014,993

(22) PCT Filed: Jul. 9, 2021
(86) PCT No.: PCT/EP2021/069162

§ 371 Date: Jan. 6, 2023

(30) Foreign Application Priority Data
Jul. 15, 2020 (EP) 20185871.9
Nov. 13, 2020 (EP) 20306378.9
Jun. 10, 2021 (EP) 21305795.3

(51) Int. Cl. C12P 19/34; C12N 9/12; C09D 11/04

(52) U.S. Cl. CPC C12P 19/34; C12N 9/1264; C12Y 207/07031; C09D 11/04

(57) ABSTRACT

The invention is directed to methods and compositions for inkjet assisted synthesis of a plurality of polynucleotides at reaction sites on a substrate using template-free polymerases, such as, terminal deoxynucleotidyl transferases (TdTs). Compositions of the invention include formulations of synthesis reagents for inkjet delivery including, but not limited to, TdT coupling reaction buffers and 3′-O-protected dNTP monomers.

### CROSS REFERENCE TO RELATED APPLICATIONS

This application is a 371 of International Application Serial No. PCT/EP2021/069162, filed on Jul. 9, 2021, which application claims priority to EP21305795.3, filed on Jun. 10, 2021, EP20306378.9, filed on Nov. 13, 2020, and EP20185871.9, filed on Jul. 15, 2020, the disclosures of which are incorporated herein by reference in their entirety.

### INCORPORATION BY REFERENCE OF SEQUENCE LISTING PROVIDED AS A TEXT FILE

A Sequence Listing is provided herewith in a text file “DNAS-025_SEQ_LIST_June_2023_ST25” created on Jun. 20, 2023, and having a size of 552.047 bytes. The contents of the text file are incorporated herein by reference in their entirety.

### BACKGROUND

Inkjet printing is a low-cost versatile technology for non-contact delivery of defined quantities of liquids to precise locations with minimal wastage, e.g. Le. Recent Progress in Ink Jet Technologies II, chapter 1 (1999); Derby. Annu. Rev. Mater. Res., 40: 395-414 (2010); Singh et al, Advanced Materials, 22: 673-685 (2010); Tekin et al, Soft Matter, 4: 703-713 (2008); Calvert, Chem Mater., 13: 3299-3305 (2001). The technology has been applied to synthesis of oligonucleotide microarrays using phosphoramidite chemistry (e.g. Brennan, U.S. Pat. No. 5,474,796; Blanchard et al. Biosensors & Bioelectronics, 11(6/7): 687-690 (1996); Hughes et al. Nature Biotechnology, 19:342-347 (2001)) and has been employed to directly print enzymes onto substrates in the production of enzyme-based biosensors, e.g. Derby, J. Mater. Chem. 18:5717-5721 (2008) Setti et al, Biosensors & Bioelectronics, 20: 2019-2026 (2005). In regard to the latter applications of inkjet printing, it has been observed that not only is enzyme activity affected by shear forces and the rheological requirements for droplet formation, but also by the changing enzyme concentration and buffer conditions from evaporative loss when, for example, enzyme-containing fluids are printed to microarrays, e.g. Derby (cited above, 2008); Di Risio et al, Macromolecular Rapid Comm., 28(18-19): (2007); Nishioka et al, J. Amer. Chem. Soc., 126(50): 16320-16321 (2004).

Recently there has been an interest in applying enzyme-based polynucleotide synthesis to problems which are ill-suited for conventional chemically based DNA synthesis, largely because of the mild aqueous reaction conditions of the enzymatic process. e.g. Church et al. Science, 10.1126/science.1226355 (2012); Lee et al. bioRxiv, http://dx.doi.org/10.1101/348987 (2018); Godron et al. International patent publication WO2020/120442. However, in addition to the above-mentioned difficulties of inkjet-delivery of enzymes, the use of enzymes presents a host of additional problems for any automated multi-step synthesis process including, enzyme adhesion to surfaces, the need for stringent temperature and pH control to maintain enzyme activity, aggregation of enzymes resulting in loss of activity and/or clogging of tubing, reaction sites or nozzles, variations in enzyme activity in or near synthesis supports, batch to batch differences in enzyme specific activity, the formation of foams or bubbles that inhibit reagent transfer and separation, loss of efficiency from reaction of certain protection groups with environmental contaminants, such as formaldehyde, and the like.

If the above challenges could be overcome, the ability to carry out inkjet reagent delivery for enzyme-based synthesis of dense arrays of polynucleotides would provide not only a convenient desk top synthesis method using aqueous reagents without the need of extensive environmental controls, but also significant advances in several diverse fields, including DNA data storage and cell and tissue analysis, such as, by direct labeling of viable biological cells, direct synthesis of spatial barcodes on tissues, and the like, e.g. Horgan et al, International patent publication WO2020/020608; Godron et al. International patent publication WO2020/120442.

### SUMMARY OF THE INVENTION

The present invention is directed to methods and compositions for inkjet-based template-free enzymatic synthesis of polynucleotides.

In some embodiments, the invention is directed to a method of enzymatically synthesizing a plurality of polynucleotides each having a predetermined sequence at distinct reaction sites on a planar substrate comprising the steps of: (a) providing a planar substrate having initiators at a plurality of distinct reaction sites, wherein each initiator has a free 3′-hydroxyl and wherein each polynucleotide of the plurality is assigned to a different reaction site for synthesis; (b) dispensing through one or more inkjet pumps at least one droplet of at least one synthesis reagent to each reaction site of the plurality to perform a reaction cycle comprising the steps of (i) reacting under elongation conditions the initiator or elongated fragments having free 3′-O-hydroxy is with a 3′-O-protected nucleoside triphosphate and a template-free polymerase so that the initiator or elongated fragments are elongated by incorporation of a 3′-O-protected nucleoside triphosphate to form 3′-O-protected elongated fragments, and (ii) deprotecting the elongated fragments to form elongated fragments having free 3′-hydroxyls, wherein the synthesis reagent comprises a template-free polymerase, a 3′-O-protected nucleoside triphosphate, a mixture of a template-free polymerase and a 3′-O-protected nucleoside triphosphate, or a deprotection solution; and (c) repeating step (b) until the plurality of polynucleotides is synthesized. In some embodiments, the polynucleotides comprise poly-2′-deoxyribonucleotides and the template-free polymerase is a template-free DNA polymerase, such as, terminal deoxynucleotidyl transferase (TdT).

In some embodiments, the invention is directed to printable template-free polymerase inks comprising: an aqueous template-free polymerase solution having a concentration of template-free polymerase in a range of from 2.0 μM to 20 μM, or from 2.0 μM to 100 μM; wherein whenever the ink is printed to a substrate, printed droplets each have a volume in the range of from 0.1 μL to 5 nL of the aqueous template-free polymerase solution and is characterized by a viscosity of about 1 centipoise to about 20 centipoise when viscosity is measured at room temperature; and a surface tension of about 15 dynes/cm and about 50 dynes/cm when measured at room temperature. In particular, such template-free polymerase inks comprise at least one terminal deoxynucleotidyltransferase (TdT) variant, a polyA polymerase (PAP) variant, or a polyU polymerase (PUP) variant. More particularly, such template-free polymerase inks comprise at least one TdT variant. In some such embodiments, the TdT inks further include a viscosity modifier. In still further embodiments, such TdT inks include a viscosity modifier and a surface tension modifier. In some embodiments, the TdT inks comprise a viscosity modifier that is also a humectant. In further embodiments, the template-free polymerase inks of the invention comprise one or more dyes. Such dyes may be fluorescent or non-fluorescent, or a mixture thereof. In some embodiments, the one or more dyes are fluorescent dyes. In some embodiments, at least one of such dyes is a separate component of an ink (which may be either a template-free polymerase ink or a non-template-free polymerase ink) for delivering a synthesis reagent, such as, a template-free polymerase, such that the dye permits optical detection to provide information as to the size, spacing and possible overlap of reaction sites, quantity of synthesis reagent delivered, and the like.

The present invention further includes the use of dye-labeled 3′-O-reversibly protected nucleoside triphosphates as monomers, wherein a coupling reaction cycle comprises the steps of (i) reacting under elongation conditions an initiator or elongated fragments having free 3′-O-hydroxyls with a base-labeled-3′-O-protected nucleoside triphosphate and a template-free DNA polymerase so that the initiator or elongated fragments are elongated by incorporation of a base-labeled 3′-O-protected nucleoside triphosphate to form 3′-O-protected elongated fragments each having a labeled base. (ii) measuring a quantity of labeled bases at each reaction site, (iii) removing labels of the labeled bases, and (iv) deprotecting the elongated fragments to form elongated fragments having free 3′-hydroxyls.

In embodiments employing 3′-O-amino-nucleoside triphosphate monomers, template, template-free polymerase inks of the invention further comprise an effective amount of an aldehyde scavenger to minimize or eliminate the generation of spuriously capped elongated fragments when adventitious aldehydes, ketones, and like compounds, react with 3′-O-amines to form oximes.

### BRIEF DESCRIPTION OF THE DRAWINGS

FIG. 1A contains a schematic representation of an enzymatic synthesis cycle wherein a 3′-O-protected dNTP is added to a nucleic acid strand followed by de-protection.

FIG. 1B illustrates a droplet microarray with a hydrophobic-hydrophilic patterned surface which may be used as a synthesis support.

FIGS. 2A-2D illustrate synthesis cycles of four different embodiments of the invention.

FIGS. 3A-3B illustrate synthesis cycles of embodiments of the invention wherein deprotection solution is dispensed by an inkjet.

FIG. 4 illustrates one embodiment of the invention where the inkjet instrument is used to define reaction sites on an array so to eliminate the problem of aligning the inkjet delivery with pre-formed reaction sites.

FIG. 5 illustrates an embodiment for synthesizing oligonucleotides, such as location tags, on a spatial cDNA library captured on a planar substrate.

FIG. 6A illustrates components of an exemplary inkjet system for use with the invention and 6B illustrates an inkjet instrument for practicing several embodiments of the invention.

FIGS. 7A-7E illustrate embodiments where a plurality of arrays of reaction sites are overlapping (or “over-written”) so that oligonucleotide barcodes may be synthesized on a substrate with a higher density than could be achieved with non-overlapping (or “non-over-written”) deposition.

FIGS. 8A-8B show formulas of exemplary O-substituted mono- and polyhydroxylamine aldehyde scavengers which may be used in methods of the invention.

FIGS. 9A-9F show data from various experiments described more fully below.

### DETAILED DESCRIPTION OF THE INVENTION

While the invention is amenable to various modifications and alternative forms, specifics thereof have been shown by way of example in the drawings and will be described in detail. It should be understood, however, that the intention is not to limit the invention to the particular embodiments described. The intention is to cover all modifications, equivalents, and alternatives falling within the spirit and scope of the invention. Guidance for selecting materials and components to carry out particular functions may be found in available treatises and references on scientific instrumentation including, but not limited to, Moore et al, Building Scientific Apparatus, Third Edition (Perseus Books, Cambridge. MA); Hiermanson, Bioconjugate Techniques, 3rd Edition (Academic Press, 2013); and like references.

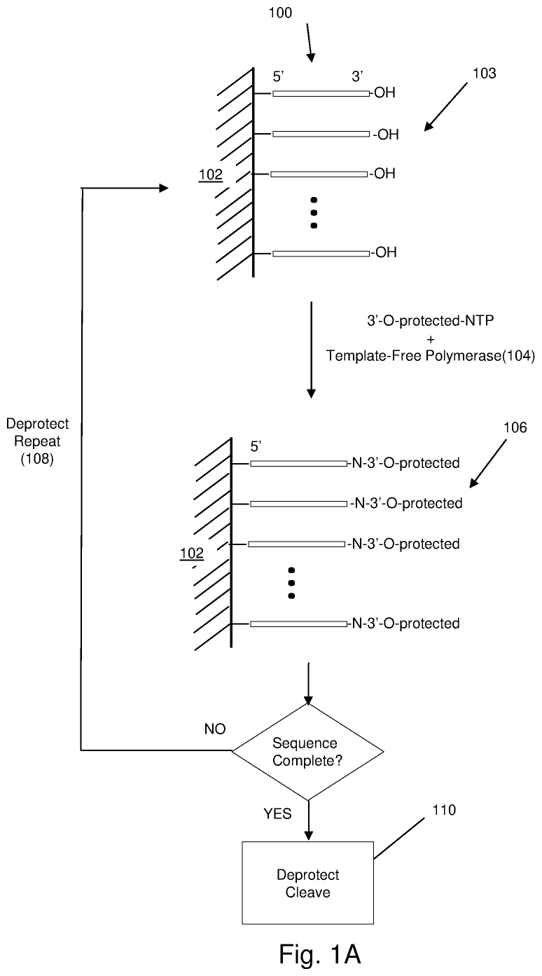

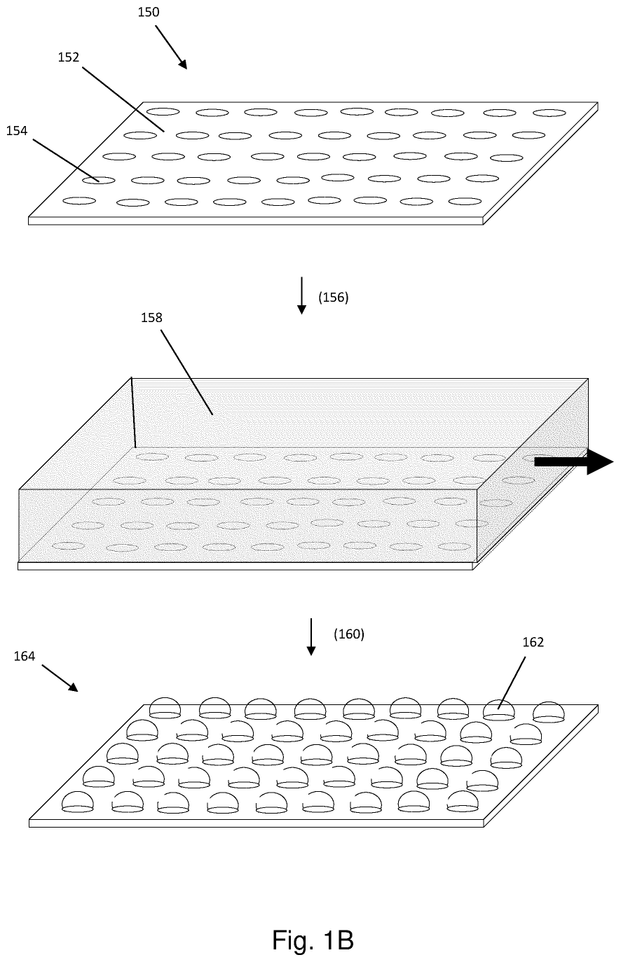

In some embodiments, the invention is directed to methods and compositions for inkjet assisted synthesis of a plurality of polynucleotides each at a distinct reaction site on a substrate using template-free polymerases, such as, terminal deoxynucleotidyl transferases (TdTs). Typically, such synthesis takes place on substrates comprising a planar surface, such as, glass, silica, silicon oxide, plastic, or like surfaces, but it may also take place on other surfaces, such as, for example, biological tissues, or surface-immobilized cDNAs extracted from tissues. As used herein, “inkjet assisted synthesis” means that one or more synthesis reagents are delivered to reaction sites in droplets generated by one or more inkjet pumps. “Synthesis reagents” include any reagent used in a synthesis cycle to couple a monomer, particularly a 3′-O-protected-nucleoside triphosphate, to an initiator or elongated fragment, such as, buffers comprising a template-free polymerase, buffers comprising 3′-O-protected-nucleotide monomers, buffers comprising a mixture of a template-free polymerase and one or more 3′-O-protected nucleoside triphosphates, deprotection (or deblocking) buffers, and the like. The terms “deprotection” agent, buffer, solution, or the like, are used synonymously herein to the terms “deblocking” agent, buffer, solution, or the like, respectively. Likewise, the term “protected” in reference to compounds, such as, dNTPs, is used synonymously with the term “blocked” in reference to compounds. As used herein, the term “deprotection solution” (or its equivalent terms) means a reagent that brings about or promotes the removal of a protection group, for example, a 3′-O-protecting group of a nucleotide. As described more fully below, the composition of a deprotection solution (and deprotection reaction conditions) depends on the nature of the protecting group (or blocking group) which is to be removed. In various embodiments, a deprotection solution may contain specific reagents that chemically react with a protection group and/or protected moiety (such as, a reducing agent like TCEP (tris(2-carboxyethyl)phosphine)), enzymes for enzymatic cleavage, scavengers, co-factors, or the like. In some embodiments, a deprotection solution may not contain specific reagents that react with a protection group, but may contain components. e.g. pH buffers, that are compatible with or promote physical cleavage of a protecting group, such as in the case of a photocleavable protecting group. Typically, in a reaction cycle for elongating a polynucleotide fragment, in a deprotecting step a deprotection solution is incubated with 3′-O-protected elongated fragments for a predetermined incubation time. Typical incubation times (i.e. durations of incubating steps) are in the range of from 1 minute to 30 minutes; or in the range of from 3 minutes to 30 minutes; or in the range of from 3 minutes to 15 minutes. Typical elongation reaction temperatures are in the range of from room temperature (RT) to 80° C.; or from 200′C to 80° C.; or from 20° C. to 60° C. “Synthesis reagents” also include reagents for preparing a substrate for polynucleotide synthesis, such as, reagents for defining reaction sites, initiators, capping reagents, and the like. Usually, a “distinct reaction site” on a substrate is a discrete site in that it is separated from other reaction sites; that is, a discrete site does not have a border with, or overlap with, another reaction site, such as exemplified in FIG. 1B. In other words, a discrete or different reaction site is not contiguous with, or overlapping, other reaction sites. Exceptions to this usual arrangement include “overwriting” embodiments described below for generating high density barcodes on surfaces.

In some embodiments, the plurality of polynucleotides may be in the range of from 2 to 500,000; or in the range of from 100 to 400,000; or in the range of from 100 to 200.000; or in the range of from 100 to 100.000. The plurality of polynucleotides may be the same or different than the plurality of reaction sites. In some embodiments, the plurality of reaction sites may be greater than the plurality of polynucleotides. In some embodiments, the above pluralities of reaction sites each have a density equivalent to that if uniformly deposited on an area equivalent to that of a standard 25 mm×75 mm microscope slide. In some embodiments, an array of reaction sites formed by uniform deposition may be a rectilinear array; and in other embodiments, an array of reaction sites formed by uniform deposition may be a hexagonal array.

The basic steps of template-free enzymatic synthesis of polynucleotides is illustrated in FIG. 1A and is described more fully below. Briefly, the synthesis process comprises cycles of steps most involving the delivery to a reaction site at least one of the following reagents: a buffer comprising a template-free polymerase, one or more buffers each comprising one or more 3′-O-protected-dNTPs (i.e. monomers), deprotection buffer, and wash solutions. In various embodiments of the invention, the template-free polymerase buffer, buffers comprising the 3′-O-protected-dNTP monomers, or deprotection buffer may be conveyed to reaction sites by droplets created and delivered by inkjet pumps. To be delivered by inkjet-generated droplets, these reagents must be formulated to meet the rheological requirements for droplet formation. These formulations are referred to as “inks.” The key rheological parameters affecting droplet formation are viscosity, density and surface tension. e.g. Derby. Annu. Rev. Mater. Sci., 40: 395-414 (2010); Derby. J. Mater. Chem., 18: 5717-57-21 (2008); Calvert. Chem. Mater., 13: 3299-3305 (2001); Tekin et al. Soft Matter, 4: 703-713 (2008); and like references. Another key parameter relating to droplet volume is the nozzle diameters of the inkjet pumps. In some embodiments, nozzle diameters for use in the present invention may be in the range of from 10 μm to 100 μm. Thus, as described more fully below, one aspect the invention includes reagent inks for template-free enzymatic synthesis of polynucleotides, and in particular, inks comprising a template-free polymerase, particularly, inks comprising a terminal deoxynucleotidyltransferase (TdT) or inks comprising a TdT and one or more 3′-O-protected-dNTP monomers. In some embodiments, inks of the invention may include more than one 3′-O-protected nucleoside triphosphates, in some cases, all four monomer types, for the purpose of synthesizing random sequence segments of polynucleotides, for example, for the creation of oligonucleotide tags or barcodes.

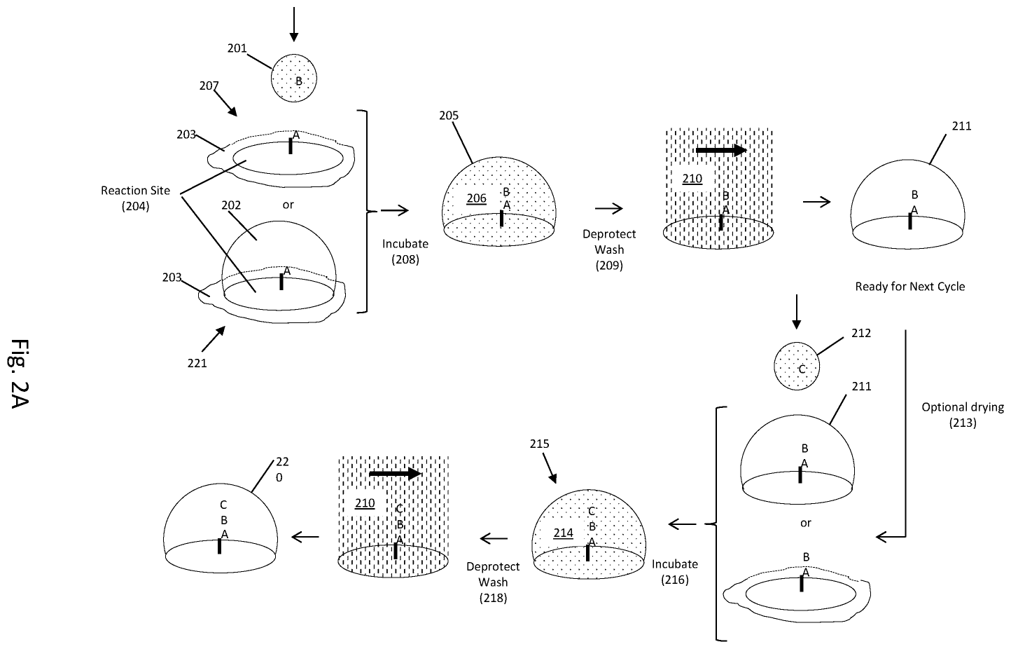

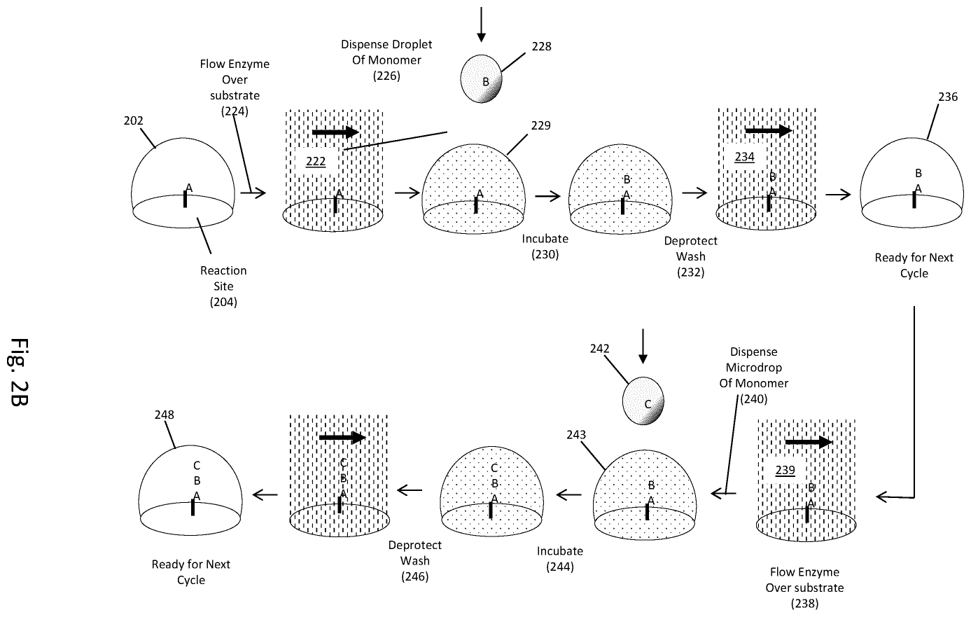

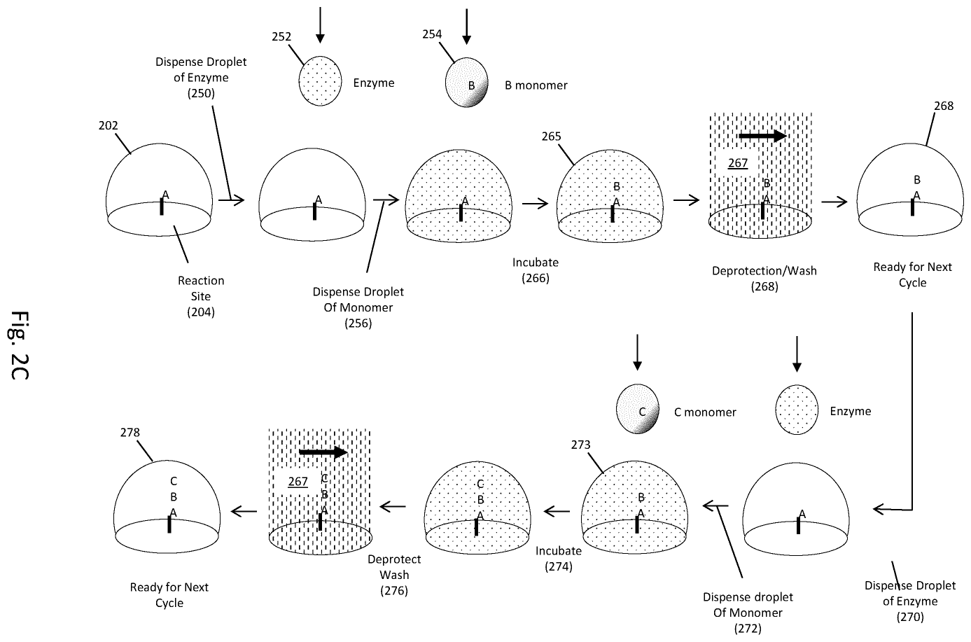

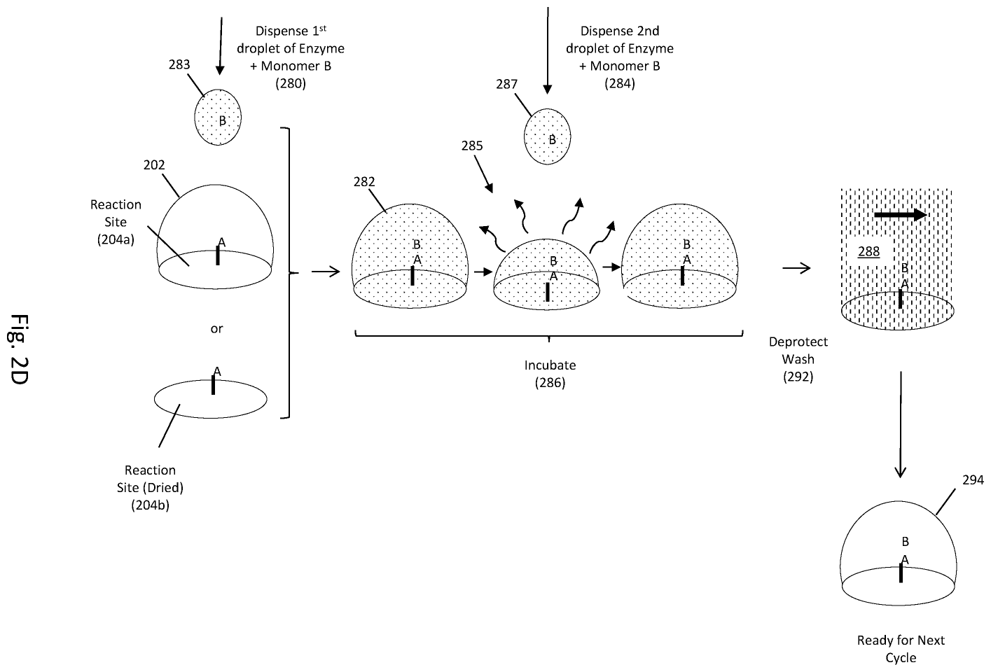

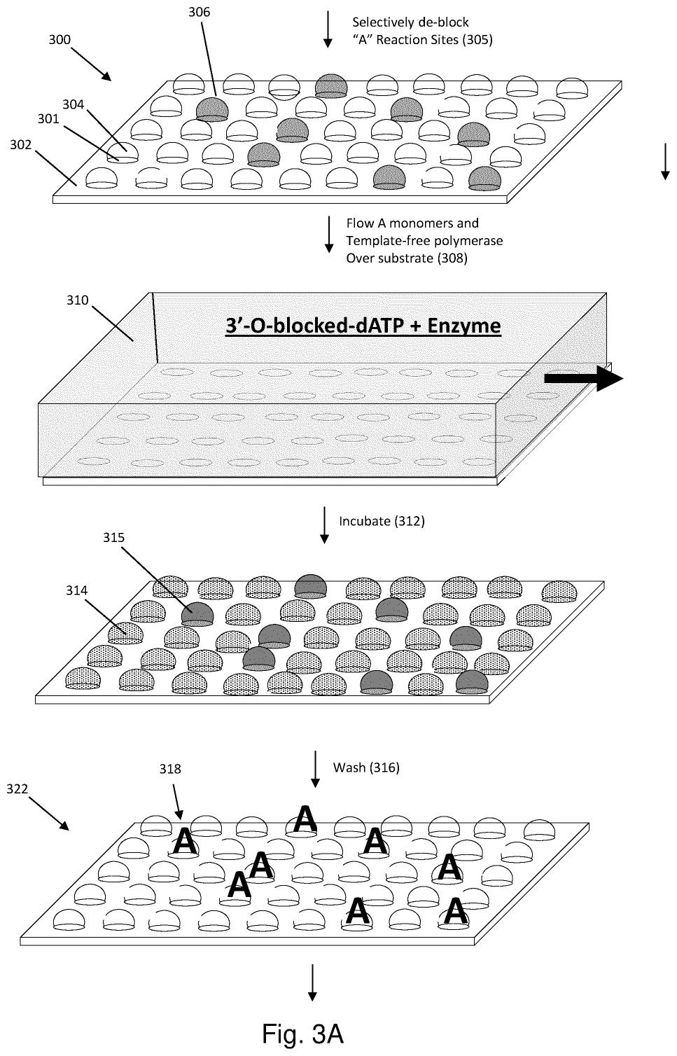

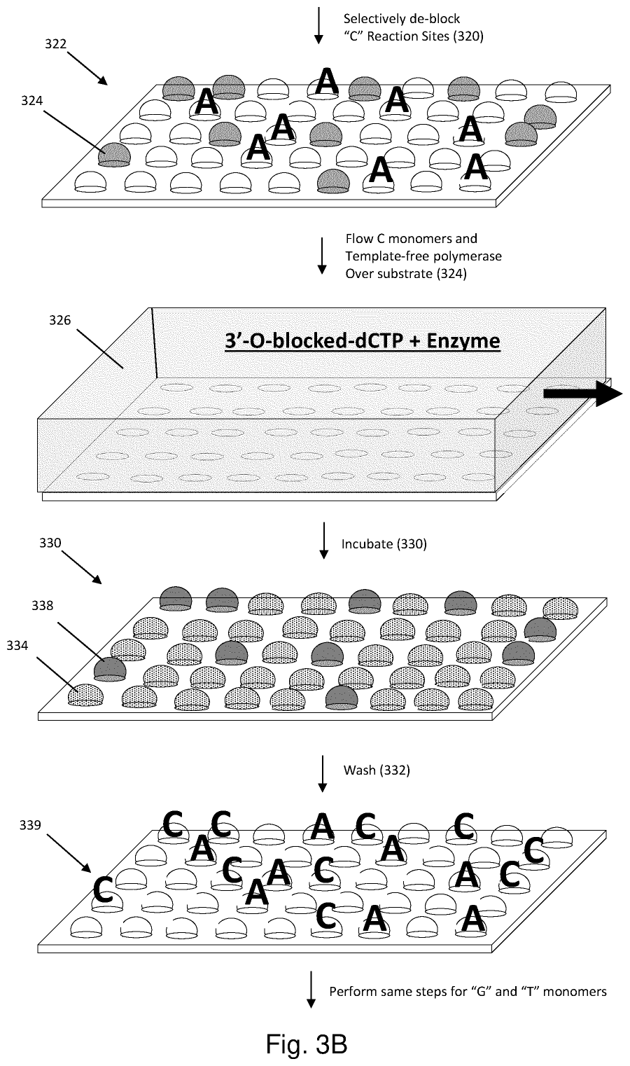

In accordance with the invention, inkjet assisted enzymatic synthesis of polynucleotides may be implemented in a variety of embodiments in which different reagents are delivered by inkjet pumps. Some of these embodiments are described in FIGS. 2A-2D and FIGS. 3A-3B. FIG. 2A illustrates two cycles in which an ink droplet (or microdrop) comprising a template-free polymerase and a single kind of monomer is delivered to a reaction site enveloped by a buffer droplet on a substrate. The surface of reaction site (204) comprises a layer of initiator oligonucleotides (not shown) and is surrounded by hydrophobic surface of substrate (203), which allows reaction site (204) to be enveloped by a volume (202) of aqueous liquid on surface (203) without spreading or coalescing with liquid from another reaction site. FIG. 2A depicts the initiators as having a 3′-terminal monomer “A” with a free 3′-hydroxyl (not shown). In FIGS. 2A-2D, monomers A, B and C are each meant to represent any 3′-O-protected-dNTP monomer. To volume (202) at reaction site (204) (or directly to reaction site (204) if a drying step is implemented), ink droplet (201) is dispensed via an inkjet pump (not shown). Ink droplet (201) comprises a predetermined concentration of a template-free polymerase and a predetermined concentration of monomer B, in addition to salts and buffer components for polymerase activity and viscosity modifiers and surface tension modifiers as needed to meet the rheological requirements for droplet formation. Droplet (201) may also include humectants to minimize evaporation loss. In some embodiments, droplet (201) may further include an aldehyde scavenger whenever 3′-O-amino-NTPs are employed. Droplet (201) is deposited on dried reaction site (207) or coalesces with volume (202) on an undried reaction site (221) to form reaction mixture (206) in droplet (205) which is allowed to incubate (208) for a predetermined time to permit coupling of B monomers to the 3′ ends of the initiators (or previously extended or elongated strands after the initial cycle). In some embodiments, such incubation takes place at a higher than ambient humidity to prevent drying during the incubation step. (In some embodiments, a separate step of drying reaction sites is implemented to prevent fluid accumulation and/or coalescence with reaction mixtures at adjacent reaction sites.) After the incubation time for the coupling reaction has elapsed, the entire substrate surface is immersed or sprayed (209) in deprotection buffer (210) for a predetermined time to permit removal of a protection group, which regenerates free 3′-hydroxyls at the ends of the elongated strands. After the predetermined deprotection time has elapsed, the entire substrate surface is immersed one or more times in one or more wash buffers for predetermined times to give reaction sites (211) with extended or elongated strands or fragments, shown as “−AB”, which are ready for the next coupling cycle. In some embodiments, as mentioned above, a drying step may be implemented after deprotection and washing (213) in order to minimize the chance of droplet (211) spreading or coalescing with adjacent droplets. Conventional drying techniques in inkjet printing may be used, warm air or gas, radiative drying, or the like, e.g. Hoynant et al, U.S. Pat. No. 8,485,096. The steps of the next cycle are the same as that for attaching the B monomer, except that in the next cycle a C monomer is coupled. Thus, droplet (212) having the same composition as droplet (201), except possibly for the identity of the C monomer, is dispensed to volume (211). As above, reaction mixture (214) in droplet (215) is incubated for a predetermined amount of time, after which the entire substrate is treated (218) with deprotection buffer and wash solution (210) to give reaction sites (220) having extended strands, illustrated as “−ABC”. Depending on the nature of substrate (203) in this and other embodiments, a coupling cycle may also include a drying steps so as to prevent droplet spreading and coalescence between adjacent reaction sites. If the surface of substrate (203) between reaction sites (204) is sufficiently hydrophobic then the possibility of such coalescence is minimized.

An embodiment of FIG. 2A may be carried out by the following steps: (a) providing a planar substrate having initiators at a plurality of distinct reaction sites, wherein each initiator has a free 3′-hydroxyl and wherein each polynucleotide of the plurality is assigned to a different reaction site for synthesis: (b) dispensing to each reaction site through one or more inkjet pumps at least one droplet a buffer solution comprising a mixture of a template-free polymerase and a 3′-O-blocked-dATP, a 3′-O-blocked-dCTP, a 3′-O-blocked-dGTP, or a 3′-O-blocked-dTTP, wherein the kind of 3′-O-blocked dNTP dispensed to a reaction site depends on the predetermined sequence of the polynucleotide assigned to the reaction site; (c) incubating the template-free polymerase and 3′-O-blocked-dNTPs at each reaction site so that initiators or elongated fragments at the reaction site are elongated by incorporation of a 3′-O-blocked dNTP to form 3′-O-blocked elongated fragments; (d) deblocking the elongated fragments at each reaction site to form elongated fragments having free 3′-hydroxyls by treating the planar support with a deblocking agent; (e) repeating steps (b). (c), and (d) until the plurality of polynucleotides is synthesized. In some embodiments, a plurality of droplet are delivered to each reaction site during each cycle of steps (b), (c) and (d). In some embodiments, the plurality of droplets is in the range of from 2 to 10, or in the range of from 2 to 5, or in the range of from 2 to 3. In other embodiments, the plurality of droplets may be in the range of from 2 to 150, or in the range of from 10 to 120. In some embodiments, a further step is implemented of cleaving the plurality of polynucleotides from the planar substrate. In some embodiments, a drying step may be included after step (d), or after step (d) and a washing step, to minimize spreading or coalescence of droplets when the next droplet is dispensed.

FIG. 2B illustrates an embodiment in which only the monomers are delivered by droplets generated by inkjet pumps. In this embodiment, the template-free polymerase is delivered by flowing a buffer containing the template-free polymerase as a bulk solution across the entire substrate. As above, the surface of reaction site (204) comprises a layer of initiators terminating with an A monomer illustrated as “−A.” Volume (202) (assuming that no drying step has been implemented) is replaced by flowing (224) enzyme buffer (222) over the entire substrate surface to form droplet (229) containing a template-free polymerase. To droplet (229) at reaction site (204), droplet (228) comprising a buffer containing B monomer is dispensed to volume (229), after which the resulting reaction mixture is allowed to incubate (230) for a predetermined amount of time. After the incubation time has elapsed, the entire surface of the substrate is immersed (232) in deprotection buffer (234) followed by one or more wash solutions to give reaction site (236) with strands extended by B monomer. The next cycle adding a C monomer operates in the same manner: flow in template-free polymerase buffer (238), dispense microdrop (242) of C monomer (240) to give reaction mixture (243), incubate (244), deprotect and wash (246) to give reaction site (248) with extended strands “−ABC.” As above, in some embodiments, a drying step may be included prior to dispensing droplets to a reaction sites, e.g. prior to (226) and (240).

An embodiment of FIG. 2B may be carried out by the following steps: (a) providing a planar support substrate having initiators at a plurality of distinct reaction sites, wherein each initiator has a free 3′-hydroxyl and wherein each polynucleotide of the plurality is assigned to a different reaction site for synthesis; (b) dispensing to each reaction site a buffer solution comprising a template-free polymerase; (c) dispensing to each reaction site through one or more inkjet pumps at least one droplet a buffer solution comprising a 3′-O-blocked-dATP, a 3′-O-blocked-dCTP, a 3′-O-blocked-dGTP, or a 3′-O-blocked-dTTP, wherein the kind of 3′-O-blocked dNTP dispensed to a reaction site depends on the predetermined sequence of the polynucleotide assigned to the reaction site; (d) incubating the template-free polymerase and 3′-O-blocked-dNTPs at each reaction site so that initiators or elongated fragments at the reaction site are elongated by incorporation of a 3′-O-blocked dNTP to form 3′-O-blocked elongated fragments; (e) deblocking the elongated fragments at each reaction site to form elongated fragments having free 3′-hydroxyls by treating the planar support with a deblocking agent; (f) repeating steps (b), (c), (d) and (e) until the plurality of polynucleotides is synthesized. As above, in some embodiments, a plurality of droplets are delivered to each reaction site during each cycle of steps (b)-(e). In some embodiments, the plurality of droplets is in the range of from 2 to 10, or in the range of from 2 to 5, or in the range of from 2 to 3. In other embodiments, the plurality of droplets may be in the range of from 2 to 150, or in the range of from 10 to 120. In some embodiments, a further step is implemented of cleaving the plurality of polynucleotides from the planar substrate. Dispensing the template-free polymerase in this embodiment can be carried out by flowing a “bulk” buffer solution containing the template-free polymerase over the entire planar substrate so that polymerase is delivered to all reaction sites in one step. As above, in some embodiments, prior to droplet dispensing steps (e.g. (250) and (256), or (270) and (272)) drying steps may be included.

FIG. 2C illustrates an embodiment in which monomer and template-free polymerase are delivered to reaction sites in separate inkjet-delivered droplets. As above, the surface of reaction site (204) comprises a layer of initiators terminating with an A monomer illustrated as “−A.” To volume (202) is dispensed (250) droplet (252) comprising a template-free polymerase, after which droplet (254) comprising B monomer is dispensed (256). In some embodiments, the order of dispensing template-free polymerase and monomer may be reversed, so that within a cycle the monomer is dispensed before the template-free polymerase is dispensed. After incubation of reaction volume (265) for a predetermined time to permit the coupling of B monomer to the initiators or extended strands at reaction site (204), the entire surface of the substrate is exposed (268) to deprotection buffer (267) and subsequently to one or more wash buffers to give reaction site (268) with extended strands “−AB.” The next cycle follows in the same manner, except C monomer is added, namely: droplet comprising template-free polymerase is dispensed (270), droplet comprising C monomer is dispensed (272) to form reaction mixture (273), incubate reaction mixture (273), deprotect and wash (276) to give reaction site (278) with extended strands “ABC.”

An embodiment of FIG. 2C may be carried out by the following steps: (a) providing a planar substrate having initiators at a plurality of distinct reaction sites, wherein each initiator has a free 3′-hydroxyl and wherein each polynucleotide of the plurality is assigned to a different reaction site for synthesis; (b) dispensing to each reaction site through one or more inkjet pumps at least one droplet a buffer solution comprising a template-free polymerase; (c) dispensing to each reaction site through one or more inkjet pumps at least one droplet a buffer solution comprising a 3′-O-blocked-dATP, a 3′-O-blocked-dCTP, a 3′-O-blocked-dGTP, or a 3′-O-blocked-dTTP, wherein the kind of 3′-O-blocked dNTP dispensed to a reaction site depends on the predetermined sequence of the polynucleotide assigned to the reaction site; (d) incubating the template-free polymerase and 3′-O-blocked-dNTPs at each reaction site so that initiators or elongated fragments at the reaction site are elongated by incorporation of a 3′-O-blocked dNTP to form 3′-O-blocked elongated fragments: (e) deblocking the elongated fragments at each reaction site to form elongated fragments having free 3′-hydroxyls by treating the planar support with a deblocking agent; (f) repeating steps (b), (c), (d) and (e) until the plurality of polynucleotides is synthesized. In some embodiments, a plurality of droplets are delivered to each reaction site during each cycle of steps (b) and (c). In some embodiments, such plurality of droplet may comprise only buffer comprising template-free polymerase, or only buffer comprising a 3′-O-blocked nucleoside triphosphate, or a combination of both. In some embodiments, the plurality of droplets is in the range of from 2 to 10, or in the range of from 2 to 5, or in the range of from 2 to 3. In other embodiments, the plurality of droplets may be in the range of from 2 to 150, or in the range of from 10 to 120. In some embodiments, a further step is implemented of cleaving the plurality of polynucleotides from the planar substrate. In some embodiments, prior to step (b) a step of drying each reaction site may be implemented, and prior to step (c) a step of drying each reaction site may be implemented.

FIG. 2D illustrates an embodiment in which multiple droplets of buffer containing template-free polymerase and monomer is delivered to a reaction site while a coupling reaction is taking place in a droplet at the reaction site. A significant problem related to carrying out reactions in droplets at a reaction site is fluid loss from evaporation. Such loss changes the concentrations of enzyme, salts, as well as components such as viscosity modifiers, surfactants, and the like, all of which may affect enzyme activity. Furthermore, in some embodiments, the concentration of template-free polymerase in a droplet delivered by an inkjet may have to be lower than that optimal for coupling reactions because at higher concentrations the polymerase increases the viscosity of the ink such that it negatively impacts droplet formation; thus, to be able to deliver by inkjet a sufficient concentration of polymerase two or more droplets of low concentration polymerase are delivered to a reaction site so that the concentration at the reaction site increases in the course of a coupling reaction by such repeated additions while at the same time buffer at the reaction site is continuously evaporating. FIG. 2D illustrates the steps of a single reaction cycle in which two droplets of a buffer comprising a template-free polymerase and a monomer are delivered to a reaction site. In some embodiments, a plurality of droplet containing such reagents may be delivered to each reaction site during synthesis. The number of such droplet deliveries is a design choice depending on factors, such as, reaction site droplet size, delivery droplet size, relative humidity, whether humectants are components of the inks, the duration of the coupling reaction, whether different print heads are used for different reagents, and the like. In some embodiments, reagents, such as, surfactants, viscosity modifiers, detergents, humectants, and the like, to alter droplets on the substrate can be delivered in separate droplets generated by separate inkjet pumps in the inkjet print head. The operation of this embodiment is similar to that of FIG. 2A, except that two or more droplets are delivered to the reaction mixture. As above, the surface of reaction site (204a, containing droplet (202), or 204b, containing no droplet) comprises a layer of initiators terminating with an A monomer illustrated as “−A.” To droplet (202) is delivered a first droplet of a buffer comprising a template-free polymerase and B monomer to form droplet (282) of reaction mixture at reaction site 1204). Again, in some embodiments employing a drying step, instead of droplet (202) at reaction site (204), droplet (283) would be delivered directly to reaction site (204h) to form reaction droplet (282). Reaction droplet (282) continuously loses water by evaporation (285) during incubation (286), which reduces its volume and increases the concentration of all the non-volatile components of the droplet, particularly that of the template-free polymerase, thereby improving coupling activity in reaction droplet (282). Coupling activity is further improved by delivering a second droplet of a buffer comprising template-free polymerase and B monomer, either separately or as a mixture. Although at the initial coalescence of delivery droplet (287) and reaction droplet (282) the concentration of polymerase is reduced, the on-going evaporation rapidly increases the concentration of polymerase in reaction droplet (282) so that it approaches a desired value. After a plurality of droplets are delivered containing buffer with polymerase and monomer, the entire surface of the substrate is immersed with deprotection buffer (288) and one or more wash solutions to give reaction site (294) with extended strands “AB”. The same procedure of delivering pluralities of droplets of reagent solutions to address problems raised by evaporation and the rheological constraints of droplet formation also can be applied to the embodiments of FIGS. 2B and 2C.

In some embodiments of FIGS. 2A-2D, coupling cycles may further include a washing step after the deprotecting step. In some such embodiments, coupling cycles may further include a drying step after a washing step. As described above, a drying step prior to a successive coupling cycle would prevent the spreading and possible coalescence of reaction droplets at adjacent reaction sites. In some embodiments, washing and drying can be combined by using a volatile wash solution, such as, acetonitrile, methanol, or the like, which is readily evaporated between coupling cycles.

In some embodiments, the droplet evaporation problem may be addressed by using a droplet-in-oil array as described by Sun et al (LabChip, 11: 2429-2436(2015)), in which aqueous reagents are delivered onto silicone oil droplets: (1) Mineral oil is first printed in a pattern on a silanized silicon dioxide surface with excellent hydrophobic and oleophobic properties using a 50 μm nozzle. (2) A first round printing of aqueous reagent A is performed on top of the preformed oil drops in the same pattern but with a 30 μm nozzle. The ejected droplets carrying reagent A penetrate the oil droplets at high velocity, overcoming their surface tension and viscosity. Subsequently, the aqueous droplets sink to the bottom of the less-dense mineral oil droplets, forming stable droplet-in-oil structures. (3) During the second-pass printing of aqueous reagents, reagent B is inkjet-printed on the just formed droplet-in-oil array of reagent A. Thus, the dispensed reagent B droplet also penetrates the oil drop due to its high velocity and immediately merges with the preformed droplet A inside the same oil drop.

FIGS. 3A and 3B illustrate an embodiment where a deprotection buffer is the synthesis reagent delivered by an inkjet generated droplet to predetermined reaction sites. Droplet microarray (300) comprises substrate (302) having a hydrophobic-hydrophilic patterned surface on which a plurality of reaction sites (e.g. 301) correspond to hydrophilic locations each of which is capable of hosting an aqueous droplet (e.g. 304). The aqueous droplets may be, for example, wash solution from a previous cycle. Alternatively, as noted above, droplets of droplet microarray (300) may be dried after exposure to such a wash solution, so that at the beginning of a cycle no droplets may be present on the “droplet” microarray. A layer of initiators or elongated fragments having protected 3′-hydroxyls is attached to the surface of each reaction site. To a number of predetermined reaction sites (e.g. 306, and others represented by darkly shaded droplets), deprotection buffer is delivered (305) by inkjet generated droplets. The predetermined reaction sites are those in which initiators or elongated fragments are to receive a 3-O-protected-dATP monomer in accordance with the predetermined polynucleotides sequences assigned to the selected reaction sites. As one of ordinary skill would appreciate, in this embodiment as well as those of FIGS. 2A-2D, monomers may have different orthogonal 3′-O-protection groups that may be removed by different deprotection buffers that permit the synthesis of multiple different polynucleotides at the same reaction site or the synthesis of DNA-RNA chimeric molecules, e.g. as described in International patent publication Godron et al. WO2020/141143. After an incubation time necessary to complete the deprotection reaction, buffer (310) comprising a template-free polymerase and 3′-O-protected-dATP is flowed across the surface of substrate (302) forming droplet microarray (320) having two types of reaction sites: those (e.g. 314) with buffer comprising template-free polymerase and monomer but without deprotected initiators or elongated fragments, and those (e.g. 315) with buffer comprising template-free polymerase and monomer and with deprotected initiators or elongated fragments. In the latter droplets only a coupling reaction takes place so that a 3′-O-protected-dATP is added to the initiators or elongated fragments at those reaction sites. After a predetermined incubation period, substrate (302) is washed (316), and optionally, dried leaving droplet microarray (322) with the initiators or elongated fragments at the selected reaction sites (e.g. 318) elongated by a 3′-O-protected-dATP.

In FIG. 3B the process is continued by delivering (320) deprotection buffer to predetermined reaction sites (e.g. 324 and others represented by darkly shaded droplets) of droplet microarray (322) so that initiators or elongated fragments at those sites are selectively deprotected. After an incubation time necessary to complete the deprotection reaction, buffer (326) comprising a template-free polymerase and 3′-O-protected-dCTP is flowed across the surface of substrate (302) forming droplet microarray (330) having two types of reaction sites: those (e.g. 334) with buffer comprising template-free polymerase and monomer but without deprotected initiators or elongated fragments, and those (e.g. 338) with buffer comprising template-free polymerase and monomer and with deprotected initiators or elongated fragments. In the latter droplets only a coupling reaction takes place so that a 3′-O-protected-dCTP is added to the initiators or elongated fragments at those reaction sites. After a predetermined incubation period, substrate (302) is washed (332), and optionally dried, leaving droplet microarray (330), or dried reaction sites, with the initiators or elongated fragments at the selected reaction sites (e.g. 339) elongated by a 3′-O-protected-dCTP. A reaction cycle is completed when similar steps are performed for coupling 3′-O-protected-dGTP and 3′-O-protected-dTTP monomers to their respective sets of reaction sites. The advantage of this synthesis approach is fewer print heads, less problems due to enzyme and higher precision since enzymatic reactions will be perfectly registered with the DNA spots.

An embodiment of FIGS. 3A-3B may be carried out by the following steps: (a) providing a planar substrate having initiators at a plurality of distinct reaction sites, wherein each initiator has a protected 3′-hydroxyl and wherein each polynucleotide of the plurality is assigned to a different reaction site for synthesis: (b) dispensing through one or more inkjet pumps to each reaction site at which a 3′-O-protected-dATP is to be coupled in accordance with the polynucleotide assigned to such reaction site at least one droplet of a buffer solution comprising a deprotection agent; (c) dispensing to the planar substrate a buffer solution comprising a template-free polymerase and a 3′-O-protected-dATP; (d) incubating the template-free polymerase and 3′-O-protected-dATP so that initiators or elongated fragments at the reaction site are elongated by incorporation of a 3′-O-protected-dATP to form 3′-O-protected elongated fragments; (e) repeating steps (b). (c) and (d) for 3′-O-protected-dCTP, 3′-O-protected-dGTP and 3′-O-protected-dTTP at their respective reaction sites; and (f) repeating steps (b). (c), (d) and (e) until the plurality of polynucleotides is synthesized. As above, in some embodiments, prior to dispensing step (b) dispensing through one or more inkjet pumps, a step of drying the reaction sites may be included.

As noted above, embodiments of the method of the invention may include one or more washing steps wherein a wash solution is flowed or sprayed on a substrate comprising an array of reaction sites. Wash solution may comprise a variety of solvents including, but not limited to, water, acetonitrile, methanol, PBS or other buffered salt solutions, or the like. In some embodiments, a wash solution may include one or more proteases. e.g. proteinase K, for the purpose of removing any polymerases that may adhere to the reaction site. That is, the embodiments of FIGS. 2A-2D and 3A-3B may further include a step of treating reaction sites with one or more proteases to remove or deactivate polymerase that may accumulate at the reaction sites.

Although FIGS. 2A-2D and 3A-3B show substrates with reaction sites continuously enveloped by, or occupied by, a droplet, this is not a requirement of all embodiments of the invention. In some embodiments substrates with reaction sites may be dried between cycles of steps so that, strictly speaking, the substrate is not always, or not continuously, a droplet microarray throughout a synthesis.

In some embodiments, including those described above, the plurality of polynucleotides enzymatically synthesized (that is, the number of reaction sites) on a substrate with inkjet delivery of reagents is in the range of from 100 to 2 million, or in the range of from 100 to 1 million, or in the range of from 100 to 100 thousand, or in the range of from 100 to 500 thousand, or in the range of from 1000 to 1 million. In some embodiments, such pluralities are synthesized on a substrate having a surface area in the range of from 1 to 500 cm², or from 1 to 256 cm², 1 to 30 cm², or having a surface area in the range of from 1 to 15 cm², or having a surface area in the range of from 1 to 7 cm², or having a surface area in the range of from 7 to 20 cm². In some embodiments, substrates may be prepare and undergo surface treatment after which it is cut, or diced, into smaller pieces for use. In some embodiments, the lengths of the polynucleotides synthesized in accordance with the invention are in the range of from 10 to 500 nucleotides, or in the range of from 50 to 500 nucleotides, or in the range of from 100 to 400 nucleotides, or in the range of from 100 to 500 nucleotides. In some embodiments, the per cycle coupling efficiency in the synthesis of polynucleotides in these length ranges is at least 98%, or is at least 99%, or is at least 99.5%, or is at least 99.8%, or is at least 99.9%. In some embodiments, the coupling cycle time in the synthesis of polynucleotides in these length ranges is less than 15 min per cycle, or less than 10 min per cycle, or less than 7 min per cycle, or less than 5 min per cycle.

In some embodiments, inkjet delivery of droplets may be directed to features on a substrate which have a dimension directly related to its size or area, such as a width of a square reaction site or a diameter of a round reaction site. Thus, in some embodiments, reaction sites have a width or diameter in the range from about 10 μm to about 1.0 cm. In some embodiments droplets can be deposited to reaction sites whose widths, or diameters, are in the range of from about 1.0 μm to about 1.0 mm, usually about 5.0 m to 500 μm, more usually about 10 μm to 200 μm, and still more usually from about 20 μm to about 100 μm.

In some embodiments, the volume of reagent ink delivered to a reaction site is in the range of 0.1 to 1000 μL, or in the range of from 0.5 to 500 μL. or in the range of from 1.0 to 250 pL, or in the range of from 1.0 to 100 μL. or in the range of from 2 to 50 pL. In some embodiments, reagent ink is delivered to each reaction site in a predetermined number of droplets, or “pulses,” generated by a print head wherein, for example, each pulse has about a 2.4 picoliter volume.

### Apparatus for Inkjet Synthesis

Delivering fluids by inkjets is a mature technology that has been available for several decades so that extensive literature is available describing it and providing guidance for adapting it to novel applications, as in the present invention. Exemplary references providing guidance for constructing inkjet delivery systems: Lausted et al, Genome Biology, 5: R58 (2004); Le, Recent Progress in Ink Jet Technologies II, chapter 1, pgs, 1-14 (1999); Derby (2010, cited above); Zapka, editor, “Handbook of Industrial Inkjet Printing.” (Wiley-VCH. Weinheim, Germany); U.S. Pat. Nos. 5,474,796; 10384189; 10669304; 6306; 6323043; 5847105; and the like. As noted by Le (1999) inkjet pumps may be classified as “continuous” and “drop-on-demand” (DOD). In some embodiments. DOD inkjet pumps are employed with apparatus of the invention, and in particular, of the various DOD inkjets, piezoelectric inkjet pumps are of interest. For example, droplet formation in DOD inkjets is described in Dong et al. Physics of Fluids, 18: 072102 (2006). Such varieties of inkjet pumps are available banks or assemblies of large numbers of inkjets (e.g. from 10's to 100's) that may be individually programmed for actuation and delivery of droplets. Such inkjets and inkjet assemblies (referred to herein as “inkjet heads”) are commercially available from many manufacturers including Epson. Xaar. Fujifilm, and the like. As used herein, “inkjet pump” means a device capable of generating and ejecting droplets of a fluid. In some embodiments, an inkjet pump is a device capable of generating and ejecting droplets of a fluid at a predetermined rate and of a predetermined uniform size. In some embodiments, an inkjet pump is capable of ejecting droplets each having approximately the same size in the range of from 0.1 μL to 5.0 nL, or the same size in the range of from 0.5 μL to 1.0 nL. In some embodiments, an inkjet pump is capable of ejecting droplets at a rate in the range of from 1 to 100 kilohertz.

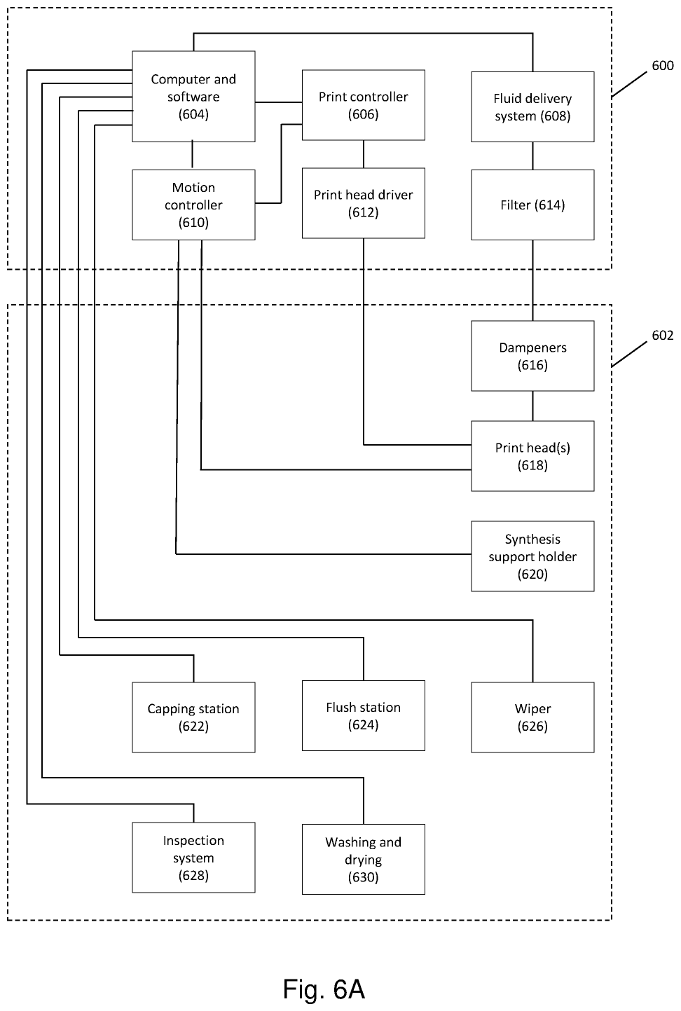

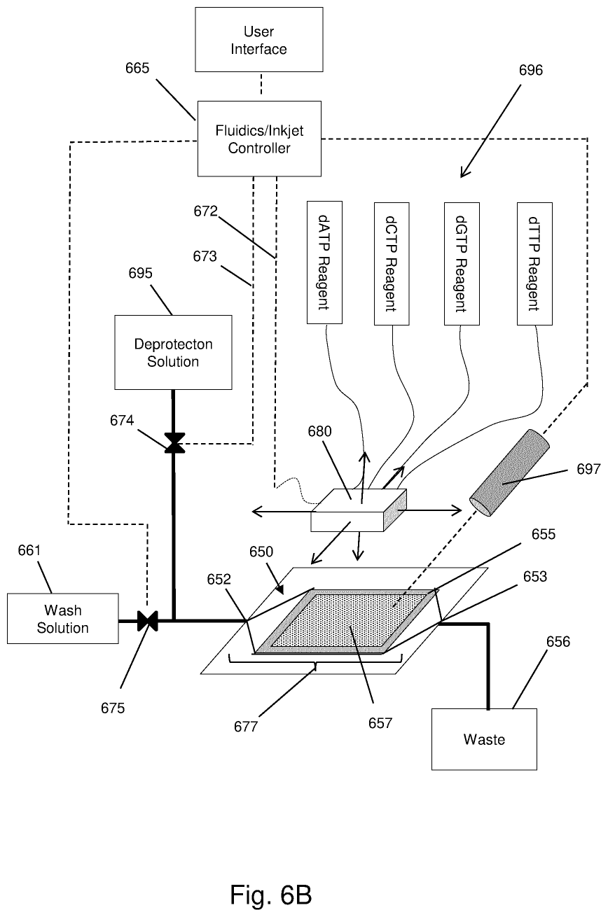

In some embodiments, components of an inkjet apparatus of the invention may be arranged according to whether they may be moved relative to one another or whether they are fixed, as illustrated in FIG. 6A by components (602) and (600), respectively. Computer and software (604) provide overall control of the system components, either directly or indirectly via controllers. For example, software may provide for single pass reagent deposition in which print head (618) is stationary and synthesis support holder (620) moves to deliver reagents to reaction sites. Alternatively, different software may provide for one or more moving print heads (618) and/or moving synthesis support holder (620) via a variety of components, such as, print controller (606), print head driver (612) and motion controller (610). Typically, computer and software (604) control capping station (622), flush station (624), wiper (626), inspection system (628) and washing and drying functions (630). Capping station (622) keeps the print head moist and stops drying of ink. Flush station (624) primes and flushes the print head, which helps remove trapped air and debris as well as dried ink. Wiper (626) is used to remove excess ink and prevent cross-contamination. It may be part of the flush station. Inspection system (628) records the presence, absence or size of spots of deposited reagents or incorrectly placed spots of reagents. Inspection system (628) may comprise a camera that takes images of the synthesis support and an image analysis software that extracts process information from the images. Such information may be used in real-time to optimize synthesis or to implement corrective measures. Washing and drying functions (630) are carried out by a fluid delivery system separate from that used for droplet delivery. Washing may include deprotection steps wherein a deprotection reagent is flowed across a synthesis substrate, optionally followed by a drying step. Drying may be accomplished by blowing air or an inert gas, such as argon, over the synthesis support, or by using a volatile solvent, such as methanol, in the washing step.

In some embodiments, cameras or microscopes may be used to capture images of the spots (i.e. reaction sites) and identify missing spots, determine spot size and spot placement. Lighting for image capture may be from above, from the side, from below or integrated into a substrate holder, whichever gives the best contrast in the absence or presence of dye in the inks. Where a dye is used (as described below), it is selected so that it would not interfere with the enzymatic reaction, would not react with the protecting group of the nucleotide and would be compatible with the enzyme and deprotection buffers. In some embodiments, each monomer would have a different distinguishable dye, covering a different part of the visible spectrum. In some embodiments, imaging of an array of reaction site is carried out during incubation (30 s-10 min) of the elongation reactions and using high enough magnification to see individual spots but not so high that an inordinate length of time would be needed to scan the array. The number of images taken in an imaging step may be 20 to 100 for a standard microscope slide. Images may be captured seamlessly in a video stream by scanning the substrate or captured in a move-stop process. The images captured may be stitched using algorithms and aided by the presence of fiducial markings on the slide. Fiducial markings also help determine whether the slide has moved in the slide holder and help determine spot positions. In some embodiments, real time image analysis allowing the identification of missing spots or poor spot placement could be accompanied by the automatic generation of a new image and an additional print or prints.

An exemplary inkjet apparatus for implementing various embodiments of the invention (e.g. those of FIGS. 2A-2D) is diagrammatically illustrated in FIG. 6B. A plurality of DOD inkjets are housed in print head (680) which is capable of x-y and z movement relative to droplet microarray (657). In some embodiments both print head (680) and droplet microarray (657) are capable of x-y movement. In some embodiments, print head (680) is held in a fixed position and droplet array (657) undergoes x-y movement. In this example. “dATP Reagent,” “dCTP Reagent,” “dGTP Reagent.” and “dTTP Reagent,” (696) are each buffer formulations, or inks, comprising template-free polymerase, respective 3′-O-protected-dNTPs, salts and cofactors necessary or useful for polymerase activity, as well as viscosity and surface tension modifiers, humectants, and the like, as needed to meet the requirements for desired droplet formation and/or to reduce evaporation loss. Print head (680) includes temperature regulation to maintain the inks at a temperature optimized for delivery and activity. In this embodiment, reagents flowed or delivered to the droplet microarray in bulk are deprotection solution (or buffer) (695) and wash solution (661). Droplet microarray (657), which is formed on substrate (655), sits or is mounted in flow chamber (677) which comprises inlet (652) and outlet (653). Flow chamber (677) defines the flow path of reagents (not delivered by print head (680)) over droplet microarray (657). Such reagents may flow continuously over droplet microarray (657) or reagents may be delivered to flow chamber (677) where they remain for a predetermined incubation time, and then are removed or recycled. Such reagents may be moved by conventional pumps or by pressure heads over reagent reservoirs. Flow chamber (677) includes temperature control elements (not shown) and humidity control elements (not shown) to maintain, or optimize, coupling reaction activity. After exiting, reagents are discarded into a waste container (656) or recycled. Timing of inkjet discharges, positioning of print head (680), actuation of valves (675) and (674) are controlled by fluidics/inkjet controller (665), which may include imaging software that performs analysis of array images obtained by camera (697) and that causes alterations of reagent deposition, for example, when coalescing reaction sites are detected. In some embodiments, print head (680) may be driven by electronics available from Meteor (Meteor Inkjet Ltd. (Cambridge. UK). For example, a Print Controller Card (PCC) synchronizes to the encoder signal from a Thorlabs motion controller. A Head Driver Card (HDC) provides power and a waveform to the printhead. The drive electronics are controlled by Meteor's digital printing front end, which includes MetDrop and MetWave software for optimization of spotting parameters, with printing initiated by the Thorlabs Kinesis software. Overall instrument control can be performed by instrument software, such as LabView.

Typically, the distance between the inkjet nozzles and the substrate surface may be in the range of from about 10 μm to 10 mm, or in the range of from about 100 μm to 2 mm, or in the range of from about 200 μm to 1 mm, or in the range of from 500 μm to 3 mm. Droplet velocities may be in the range 1-10 meters/sec. Print head movement may be in the range of from 1-30 cm/sec, or 5-30 cm/sec. or 20-30 cm/sec. As described more fully below, print heads may have different droplet delivery modes, for example, single-pass mode, multiple pass mode, and move-stop mode.

As mentioned above, in some embodiments, nozzle diameters for use with the invention may be in the range of from 10 μm to 100 μm. In other embodiments, inkjet nozzle size may be in the range of from 20-30 μm for generating droplet sizes in the range of from 10-20 μL. In some embodiments, nozzle diameter, synthesis reagent density, surface tension and viscosity are selected to dispense droplets to reaction sites having a volume in the range of from 2 μL to 5 nL, or in the range of from 2 μL to 1 nL, or in the range from 2 μL to 500 μL, or in the range from 2 μL to 100 μL. In some embodiments, inkjet pumps are DOD inkjet pump and have a droplet generation rate in the range of from 1 to 100 kHz.

In some embodiments, inkjet-based synthesizers include droplet detection components to monitor and record any anomalies in droplet formation and delivery by the inkjet nozzles. In some embodiments, such droplet monitoring may comprise a laser diode mounted orthogonally to the direction of print-head motion such that the droplet stream of each bank of nozzles intersects the beam, causing the light to scatter if a droplet is present. Before each round of printing, nozzles may be fired in series through the beam and the forward scattering of each droplet is detected by a photodiode. Nozzles failing to fire may be taken off-line during synthesis. Apparatus of the invention may also be equipped with commercially available droplet monitors, such as, a Meteor dropwatcher, available from Meteor Inkjet Ltd, (Cambridge. UK) as well as a camera to image the solid support and array of reaction sites. The latter permits the array of reaction sites to be monitored to detect accuracy in droplet deposition, size and geometry of reaction sites, coalescence of reaction sites, and the like. In some embodiments, software may be provided to provide a full image of an array on a slide or solid support by patching together tiles comprising smaller images, e.g. S. Preibisch, S. Saalfeld, P. Tomancak, Bioinformatics, 2009, 25(11), 1463-1465.

In certain embodiments, it may be desirable to prevent evaporation of the synthesis reagents and reaction mixtures following deposition. Evaporation may be prevented in a number of different ways. In some embodiments, synthesis cycles may be carried out in a high humidity environment, such as a relative humidity in the range of from 75-85%. Alternatively or in addition to, one may employ reagents with an evaporation retarding agent or humectant. e.g. glycerol, polyethylene glycol, carboxymethyl cellulose, hydroxyethyl cellulose, and the like.

In some embodiments, recirculating ink print heads are employed because problems of drying and/or clogging of nozzles by enzymes is reduced. Recirculating ink print heads are commercially available, for example, from Fujifilm and are described in U.S. Pat. No. 8,820,899; 8534807; 8752946; 9144993; 9511598; 9457579, which are incorporated herein by reference.

### Synthesis Substrates

In some embodiments, substrates for synthesis comprise surfaces that have been patterned with hydrophobic and hydrophilic regions wherein discrete hydrophilic reaction sites are formed. These allow the formation of droplets on hydrophilic reaction sites, for example, after flowing aqueous reagents or reactants of the entire surface. That is, in some embodiments, substrates for synthesis comprise so-called “droplet microarrays,” e.g. as disclosed in the following exemplary references, which are incorporated by reference: Brennan, U.S. Pat. No. 5,474,796: Chrisey et al. Nucleic Acids Research, 24(15): 3040-3047 (1996); Fixe et al, Materials Research Society Symposium Proceedings. Volume 723, Molecularly Imprinted Materials—Sensors and Other Devices. Symposia (San Francisco. California on Apr. 2-5, 2002); Goldfarb. U.S. patent publication 2008/0166667; Gopinath et al, ACS Nano, 8(12): 12030-12040 (2014); Hong et al, Microfluid. Nanofluid., 10: 991-997 (2011); Kumar et al, Nucleic Acids Research, 28(14): e71 (2000); Peck et al, U.S. patent Ser. No. 10/384,189; Indermuhle et al, U.S. patent Ser. No. 10/669,304; Wu et al, Thin Solid Films, 515: 4203-4208 (2007); Zhang et al, J. Phys. Chem., 111: 14521-14529 (2007): and like references. As used herein, the term “droplet microarray” refers to a planar substrate whose surface has been treated to create a plurality of discrete hydrophilic regions, which may serve as reaction sites either directly or with further treatment. e.g. attaching initiators. In some embodiments, each of the plurality of discrete hydrophilic regions are surrounded by hydrophobic regions. The discrete hydrophilic regions may have a variety of shapes, but are usually circular or rectangular or square for manufacturing convenience. In some embodiments, reaction sites have areas and capacities to hold an aqueous reaction mixture as described above. Although synthesis substrates of some embodiments may comprise droplet microarrays, in a synthesis process such arrays may undergo a drying step which removes liquid from reaction sites. That is, in some embodiments, a synthesis substrate comprising a droplet microarray may be devoid of droplets from time to time, for example, after an elongation cycle ending in a drying step. The hydrophilic-hydrophobic configurations permit the formation of droplets on the surface of a droplet microarray either after inkjet delivery of a synthesis reagent to the hydrophilic regions or by flowing a “bulk” aqueous solution, such as a synthesis reagent or wash solution, over the substrate. As disclosed in the above references, the droplets retained by the hydrophilic regions may serve as reaction chambers or vessels. Such a process is illustrated in FIG. 1B. Planar substrate (150) has a surface with hydrophobic region (152) and discrete hydrophilic regions (154) which may serve as reaction sites. When planar substrate (150) is flooded (156) with aqueous solution (158) both hydrophobic regions (152) and hydrophilic regions (154) are immersed. When aqueous solution (158) drains off (160) some of the aqueous solution is retained by hydrophilic regions (154) to form droplets (162) of droplet microarray (164). Individual droplets, such as (162), may be referred to as “microarray droplet” to distinguish them from droplets formed by an inkjet pump prior to its delivery to a reaction site, such as (162).

Preparation of substrates with discrete reaction sites can be accomplished by known methods. For example, such methods can involve the creation of hydrophilic reaction sites by first applying a protectant, or resist, over selected areas over the surface of a substrate, such as a silicon oxide, or like material. The unprotected areas are then coated with a hydrophobic agent to yield an unreactive surface. For example, a hydrophobic coating can be created by chemical vapor deposition of (tridecafluorotetrahydrooctyl)-triethoxysilane onto the exposed oxide surrounding the protected circles. Finally, the protectant, or resist, is removed exposing the well regions of the array for further modification and nucleoside synthesis using the high surface tension solvents described herein and procedures known in the art such as those described by Maskos & Southern, Nucl. Acids Res. 20:1679-1684 (1992). Alternatively, the entire surface of a glass plate substrate can be coated with hydrophobic material, such as 3-(1,1-dihydroperfluoroctyloxy)propyltriethoxysilane, which is ablated at desired loci to expose the underlying silicon dioxide glass. The substrate is then coated with glycidyloxypropyl trimethoxysilane, which reacts only with the glass, and which is subsequently “treated” with hexaethylene glycol and sulfuric acid to form an hydroxyl group-bearing linker upon which chemical species can be synthesized (Brennan, U.S. Pat. No. 5,474,796). Arrays produced in such a manner can localize small volumes of solvent within the reaction site by virtue of surface tension effects (Lopez et al., Science 260:647-649 (1993)).

In some embodiments, reaction sites may be formed on a substrate following the photolithographic methods of Brennan, U.S. Pat. No. 5,474,796; Peck et al, U.S. patent Ser. No. 10/384,189; Indermuhle et al, U.S. patent Ser. No. 10/669,304; Fixe et al (cited above); or like references cited above. In accordance with these methods, a set of hydrophilic molecules comprising an aminosilane is attached to the surface of a substrate to form reaction sites. Such hydrophilic molecules may comprise N-(3-triethoxysilylpropyl)-4-hydroxybutyramide (HAPS), 11-acetoxyundecyltriethoxysilane, n-decyltriethoxysilane, (3-aminopropyl)trimethoxysilane, (3-aminopropyl)triethoxysilane, 3-glycidoxypropyltrimethoxysilane (GOPS), or 3-iodo-propyltrimethoxysilane. A set of hydrophobic molecules comprising a fluorosilane is attached to the surface of the substrate in regions outside of the reaction sites. Such hydrophobic molecules may comprise perfluorooctyltrichlorosilane octylchlorosilane, octadecyltrichlorosilane. (tridecafluoro-1,1,2,2-tetrahydrooctyl)trichlorosilane, or tridecafluoro-1,1,2,2-tetrahydrooctyl)trimethoxysilane. After such attachment, a substrate is prepared for polynucleotide synthesis by coupling initiators to the aminosilanes at the reaction sites. Such coupling may be accomplished using any number of available homo- or heterobifunctional linkers to form covalent bonds between amino groups on the substrate and 5′-thiol groups or 5′-amino groups on the initiators. Such linkers are, for example, available from Sigma-Aldrich (St. Louis, MO) and are described in treatises such as, Hermanson. Bioconjugate Techniques, 3^{rd} Edition (Academic Press, 2013). Synthesis of oligonucleotides having 5′-thiol or 5′-amino groups is well-know and is described in Kupihar et al, Nucleosides Nucleotides & Nucleic Acids, 22(5-8): 1297-1299 (2003); Fung et al, U.S. Pat. No. 4,757,141; and like references.

In some embodiments, an array of reaction sites may be formed using click chemistry by depositing under coupling conditions droplets of 5′-DBCO (dibenzocyclooctyl) labeled initiators (e.g. Glen Reseach) on a planar substrate comprising an azide layer (e.g. PolyAn 2D azide glass slide). In some embodiments, such reactions may be carried out as a copper-free click reaction which is less damaging to the DNA. e.g. Donnerholt et al. Top. Curr. Chem. (Z) 374: 16 (2016).

A wide variety of substrates may be employed for creating arrays of reaction sites for enzymatic synthesis of polynucleotides. Substrates may be a rigid material including, without limitation, glass; fused silica; silicon such as silicon dioxide or silicon nitride; metals such as gold or platinum; plastics such as polytetrafluoroethylene, polypropylene, polystyrene, polycarbonate, and any combination thereof. A rigid surface can be fabricated from a material selected from the group consisting of silicon, polystyrene, agarose, dextran, cellulosic polymers, polyacrylamides, polydimethylsiloxane (PDMS), and glass. Substrates may also comprise flexible materials, which is capable of being bent, folded or similarly manipulated without breakage. Exemplary flexible materials include, without limitation, nylon (unmodified nylon, modified nylon, clear nylon), nitrocellulose, polypropylene, polycarbonate, polyethylene, polyurethane, polystyrene, acetal, acrylic, acrylonitrile, butadiene styrene (ABS), polyester films such as polyethylene terephthalate, polymethyl methacrylate or other acrylics, polyvinyl chloride or other vinyl resin, transparent PVC foil, transparent foil for printers. Poly(methyl methacrylate) (PMMA), methacrylate copolymers, styrenic polymers, high refractive index polymers, fluorine-containing polymers, polyethersulfone, polyimides containing an alicyclic structure, rubber, fabric, metal foils, and any combination thereof.

In some embodiments, patterned surfaces of superhydrophobic and superhydrophilic regions may be formed on a substrate. Guidance for forming droplet microarrays with such patterned surfaces are described in the following references, which are incorporated by reference: Feng et al, Adv. Mater. Interfaces, 1400269 (2014); Zhan et al. Trends Anal. Chem., 108: 183-194 (2018); Neto et al, Adv. Functional Mater., 201400503 (2014);

Achieving accurate alignment of droplet delivery to reaction sites of a prefabricated droplet microarray is an important aspect of inkjet-assisted synthesis of polynucleotides. In some embodiments, such alignment tasks may be minimized or avoided by creating immediately prior to synthesis an array of reaction sites by depositing droplets of synthesis reagents onto a layer of initiator oligonucleotides on a substrate in order to define the locations of reaction sites. Following this initial deposit of droplets, the initiator layer outside of the droplet-defined sites are treated to render them inert to subsequent extension or to render them inert to extension as well as hydrophobic. After such an initial surface treatment to create reaction sites, further or subsequent inkjet delivery of droplets to the same reaction sites will be accurate because the same inkjet head and pumps that were used to define the locations of the reaction sites will be used to deliver subsequent droplets during synthesis of the polynucleotides. In some embodiments, the synthesis reagents delivered to the initiator layer comprise a mixture of a template-free polymerase and a 3′-O-protected-dNTP. These reagents extend the initiators to define reaction sites or regions on the oligonucleotide layer which is populated by extended fragments having 3′-O-protected ends. The areas outside of these regions are then treated to render them inert to extensions. In some embodiments, after the initial coupling step defining reaction sites, the entire substrate is exposed to a template-free polymerase and a terminator, such as a dideoxynucleoside triphosphate (ddNTP), or like reagent. In some embodiments, such ddNTP could be, for example, a ddNTP conjugated to a hydrophobic moiety, thereby rendering the coating outside of the reaction sites hydrophobic. Such a hydrophobic moiety may be, for example, a dye or quencher molecule, such as, a Black Hole Quencher® molecule. A variety of terminators may be employed for this purpose. In particular, terminators include nucleoside triphosphates that lack a 3′-hydroxyl substituent and include 2′,3′-dideoxyribose, 2′,3′-didehydroribose, and 2′,3′-dideoxy-3′-haloribose, e.g. 3′-deoxy-3′-fluoro-ribose or 2′,3′-dideoxy-3′-fluororibose nucleosides. Alternatively, a ribofuranose analog can be used in terminators, such as 2′,3′-dideoxy-p-D-ribofuranosyl, β-D-arabinofuranosyl, 3′-deoxy-β-D-arabinofuranosyl, or the like. Further terminators are disclosed in the following references: Chidgcavadze et al., Nucleic Acids Res., 12: 1671-1686 (1984); Chidgcavadze et al., FEBS Lett., 183: 275-278 (1985); Izuta et al, Nucleosides & Nucleotides, 15: 683-692 (1996); and Krayevsky et al, Nucleosides & Nucleotides, 7: 613-617 (1988). Nucleotide terminators also include reversible nucleotide terminators, e.g. Metzker et al. Nucleic Acids Res., 22(20):4259 (1994).

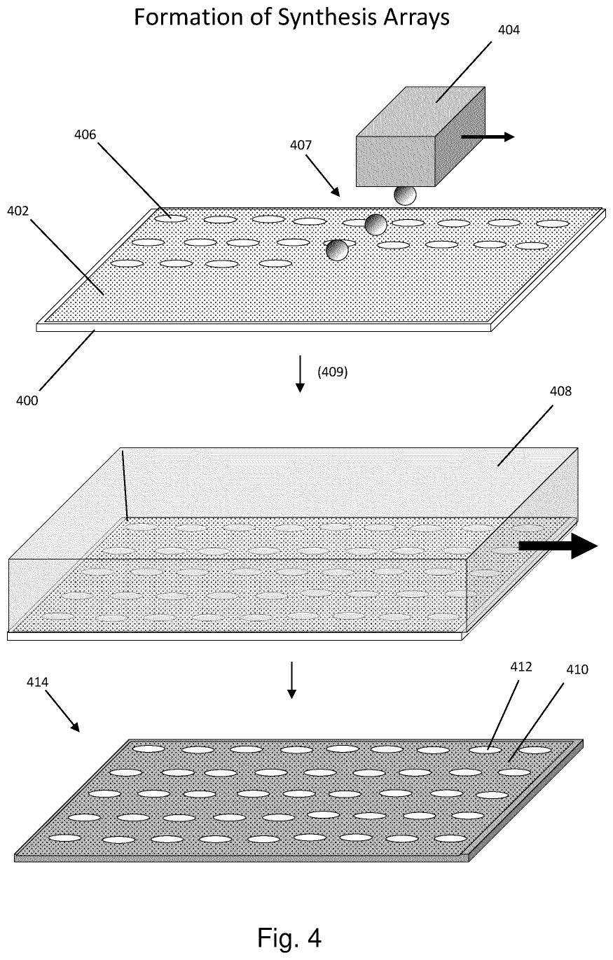

Thus, in such embodiments, a starting material for a synthesis operation is a surface coated with a layer of initiator oligonucleotides. An exemplary fabrication of reaction site on such starting material is illustrated in FIG. 4. Planar substrate (400) (e.g. a glass slide) has a layer (402) of initiator oligonucleotides that have free 3′-hydroxyl groups and that are attached by their 5′-ends to the substrate. In some embodiments, initiator densities may be, for example, in the range of from 10¹¹ to 10¹³ strands/cm². Inkjet pumps in inkjet head (404) are used to deposit droplets (407) in a regular and repeatable pattern on layer (402) that define reaction sites (e.g. 406). For example, the 3′-hydroxyls of such initiators may be unprotected and the droplets may contain a template-free polymerase and an initial 3′-O-protected nucleoside triphosphate, thereby producing 3′-O-protected elongated fragments in each reaction site. After such deposition, the layer (402) of initiators is immersed and incubated (409) in a buffer (408) comprising a template-free polymerase and a terminator. e.g. as described above, to produce droplet microarray (414) having a surface (410) outside of the reaction sites (e.g. 412) inert to extension or inert to extension and hydrophobic depending on the terminator selected.

An embodiment of the invention for synthesizing a plurality of polynucleotides employing reaction site formation as described in FIG. 4 may be carried out by the following steps: (a) providing a planar substrate having attached a layer initiators, wherein each initiator has a free 3′-hydroxyl; (b) dispensing through one or more inkjet pumps one or more droplets to each of a plurality of sites on the layer of initiators to define an array of reaction sites, wherein each droplet comprises a buffer solution comprising a mixture of a template-free polymerase and a 3′-O-blocked-dATP, a 3′-O-blocked-dCTP, a 3′-O-blocked-dGTP, or a 3′-O-blocked-dTTP, and wherein each polynucleotide of the plurality is assigned to a different reaction site for synthesis; (c) capping the free 3′-hydroxyls of initiators outside of the reaction sites; (d) dispensing to each reaction site through one or more inkjet pumps at least one droplet a buffer solution comprising a mixture of a template-free polymerase and a 3′-O-blocked-dATP, a 3′-O-blocked-dCTP, a 3′-O-blocked-dGTP, or a 3′-O-blocked-dTTP, wherein the kind of 3′-O-blocked dNTP dispensed to a reaction site depends on the predetermined sequence of the polynucleotide assigned to the reaction site; (c) incubating the template-free polymerase and 3′-O-blocked-dNTPs at each reaction site so that initiators or elongated fragments at the reaction site are elongated by incorporation of a 3′-O-blocked dNTP to form 3′-O-blocked elongated fragments; (f) deblocking the elongated fragments at each reaction site to form elongated fragments having free Y-hydroxyls by treating the planar support with a deblocking agent: (g) repeating steps (d), (e), and (f) until the plurality of polynucleotides is synthesized.

An aspect of the invention is a method for preparing an array of reaction sites for template-free enzymatic synthesis of a plurality of polynucleotides. In some embodiments, such method of array preparation may be carried out by the steps of (a) providing a surface with initiators attached. (b) delivering with one or more inkjet pumps droplets to a plurality of distinct locations on the surface to form a plurality reaction sites, the droplets containing a synthesis reagent that reacts with initiators in the reaction sites to remove 3′-O-protecting groups or to elongate such initiators by addition of a 3′-O-protected nucleoside triphosphate, and (c) capping initiators on the surface outside of the reaction sites. In some embodiments, initiators on the surface of step (a) have free 3′-hydroxyls and the synthesis reagent delivered in step (b) comprises a template-free polymerase and a 3′-O-protected nucleoside triphosphate, so that the template-free polymerase catalyzes the addition of the 3′-O-protected nucleoside triphosphate to produce 3′-O-protected elongated fragments within the reaction sites. Thus, initiators outside of the reaction sites may be capped by immersion of the surface in a capping reagent (such as a mixture containing a dideoxynucleoside triphosphate and template-free polymerase). In some embodiments, initiators on the surface may have 3′-O-protection groups and the synthesis reagent delivered by droplets may contain a deprotection agent that removes the 3′-O-protection groups from initiators to form reaction sites. In the newly formed reaction sites, a reagent is delivered which contains 3′-O-protected nucleoside triphosphates and a template-free polymerase, wherein the protection group of the delivered nucleoside triphosphate is orthogonal to that of the initiators of the surface. Exemplary orthogonal 3′-O-protection groups are described below. For example, such orthogonal protection groups may be azidomethyl and amino.

One of ordinary skill would appreciate that similar reaction site formation can be implemented for other embodiments, such as those described in FIGS. 2B and 2C. One of ordinary skill would also appreciate that the optional steps (e.g. washing, drying, treating with protease, or the like) described for the embodiments of FIGS. 2A-2D and FIGS. 3A-3B may also be implemented in the embodiments described above, including those of FIG. 4.

In another embodiment, a beginning layer of initiator oligonucleotides all have 3′-O-amino-protected or 3′-O-azidomethyl-protected ends. The process steps in this embodiment are similar to those of FIG. 4, except that a deprotection buffer is inkjet printed on the substrate to define reaction sites as discrete regions of initiators having free 3′-hydroxyls. After such selective deprotection, the surface is treated with an aqueous solution of an aldehyde or ketone to form a stable non-extendable hydrophilic or hydrophobic 3′-oxime. The aldehyde or ketone could be water soluble. e.g. acetone, or slightly water soluble and hydrophobic (e.g. pentanal, aldehyde-PEG-DBCO, or the like) or very hydrophobic and water insoluble (e.g. heptanal).

In another embodiment, a buffer comprising a template-free polymerase/3′-O-protected-dNTP mixture is printed on the initiator oligonucleotide layer with free 3′-hydroxyls as described above to define reaction sites having extended initiators with 3′-O-protected ends. The surface outside these defined sites is then treated with template-free polymerase and azide or alkyne derivatized ddNTP to block further 3′ extensions. A hydrophobic molecule with a complementary click chemistry group (e.g. DBCO, benzyl-azide) may then be reacted with the ddNTP terminator to render the surface outside of the reaction sites hydrophobic. Exemplary click chemistry pairs are described in Feng et al. Adv. Mater. Interfaces, 1400269 (2014).

In still another embodiment, to a substrate surface without a layer of initiator oligonucleotides, a buffer comprising initiator oligonucleotides having 5′ linker groups is inkjet printed on the surface derivatized with a complementary reactive group (e.g. epoxy, azide/alkyne so that the initiators are attached to the surface by their 5′-ends. To these attached initiators, cycles of coupling reactions can take place in accordance with the invention. Also, unreacted complementary reactive groups may be quenched by reacting them with an inert group (e.g. ethanolamine for epoxy) and the inert group may be selected to have a hydrophobic character.

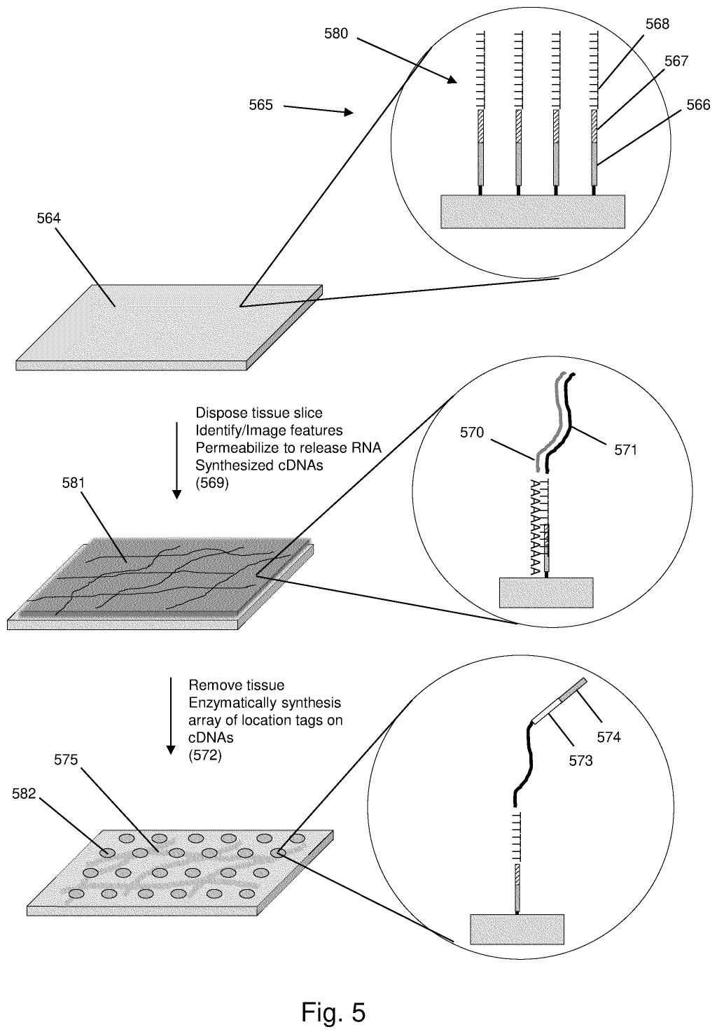

In some embodiments, substrates for synthesis may include surface-bound cDNAs copied from messenger RNA extracted from a fixed or non-fixed tissue slice. Procedures for placing tissue slices on a planar array of oligonucleotides, identifying and imaging tissue features (such as cell boundaries), permeablizing cells of tissues, implementing reverse transcriptase reactions to produce a cDNA library attached to a planar array are disclosed in Stahl et al, Science, 353: 78-82 (2016); and Frisen et al, U.S. Pat. No. 9,593,365 and 10030261; and like references, which are incorporated herein by reference. Briefly, referring to FIG. 5, planar array (564) is provided with a uniform coating of oligonucleotides (580), with a controlled density, or predetermined density, attached by their 5′ ends, wherein the oligonucleotides (shown in magnified view (565)) comprise segment (566), such as a primer binding site, for later amplification and manipulation of a cDNA, optional segment (567) comprising a molecular tag (sometimes referred to as a “unique molecular identifier” or UMI) which facilitates quantification of cDNA molecules even after amplification, and segment (568), such as a polyT segment, which permits capture of mRNA released from cells. The UMI (567) may comprise a random nucleotide segment. Oligonucleotides (580) may be made in bulk using conventional techniques and applied to the surface of planar array (564) in a single step. Different kinds of oligonucleotides, for example, oligonucleotides with different position tags are not required. Segment (567) may also include a cleavable linker or cleavable nucleotide for releasing cDNAs for analysis, such as, by sequencing. Onto array (564) is disposed a slice or thin layer (581) (e.g. 100-1000 μm thick) of tissue, which it is then treated (569) (i) to identify features, such as cells or sub-tissues, of interest and to record and/or correlate such information to locations on planar array (564), and (ii) to permeabalized cells in the tissue so that mRNA is released and allowed to diffuse to and be captured by oligonucleotides (580).

The image information is used to define regions on array (564) within which common position tags are synthesized on cDNAs. Treatments may include staining with tissue-specific or biomolecule-specific compounds or dyes. The position tags allow cDNAs to be harvested and sequenced in bulk, yet be related to specific regions by their position tags. After the above steps (i) and (ii), reagents for a reverse transcriptase reaction are applied in order to synthesize cDNAs (571) using captured mRNAs (570) as templates to produce a spatial cDNA library array. Tissue slice (581) is then removed leaving array (564) with a pattern of different cDNAs attached to its surface. The different cDNAs at the different positions may be identified and quantified by attaching position tags to samples of cDNAs from a plurality of locations by inkjet delivery of synthesis reagents for the tags, which is illustrated in FIG. 5 by the superposition of synthesis locations (582) on cDNA pattern (575). In some embodiments, such plurality may be at least 100 positions, or at least 1000 positions, or at least 10.000 positions; in other embodiments, such plurality may be in the range of from 10 to 50.000 positions; or from 10 to 10.000 positions; or from 10 to 1000 positions. Guidance for design and control of inkjet delivery systems is well known by those with skill in the art and may be found in U.S. patent publication US2003/0170698 and U.S. Pat. No. 6,306,599; 6323043; 7276336; 7534561; and like references. Position tags (573) are selected (e.g. are long enough) to uniquely identify each location or region of interest. Additional segment (574) may be added to facilitate manipulation and sequencing of cDNAs (571).

In some embodiments, this application of the invention may be carried out with the following steps: (a) providing an array comprising a uniform coating of capture probes each comprising a capture segment; (b) contacting a tissue sample with the array and allowing the nucleic acid of the tissue sample to interact with the capture domain of the capture probe so that the nucleic acid is captured; (c) treating the tissue sample to identify different regions of the tissue sample; (d) generating a nucleic acid molecule from the nucleic acid that interacts with the capture domain; (e) enzymatically synthesizing position tags onto the nucleic acid molecules; (f) determining the region that is associated with the nucleic acid that interacts with the capture domain; and (e) correlating the determined regions to the cDNAs. In some embodiments, the nucleic acid molecules from the tissue sample is RNA. In other embodiments, the nucleic acid molecules from the tissue sample may be genomic DNA. In other embodiments, the nucleic acid molecules from the tissue sample may be mRNA. In some embodiments, the step of enzymatically synthesizing position tags onto the nucleic acid molecules is carried out by inkjet delivery of synthesis reagents to the locations of the position tags in accordance with methods of the present invention.

### Overwriting for High Density Barcode Synthesis

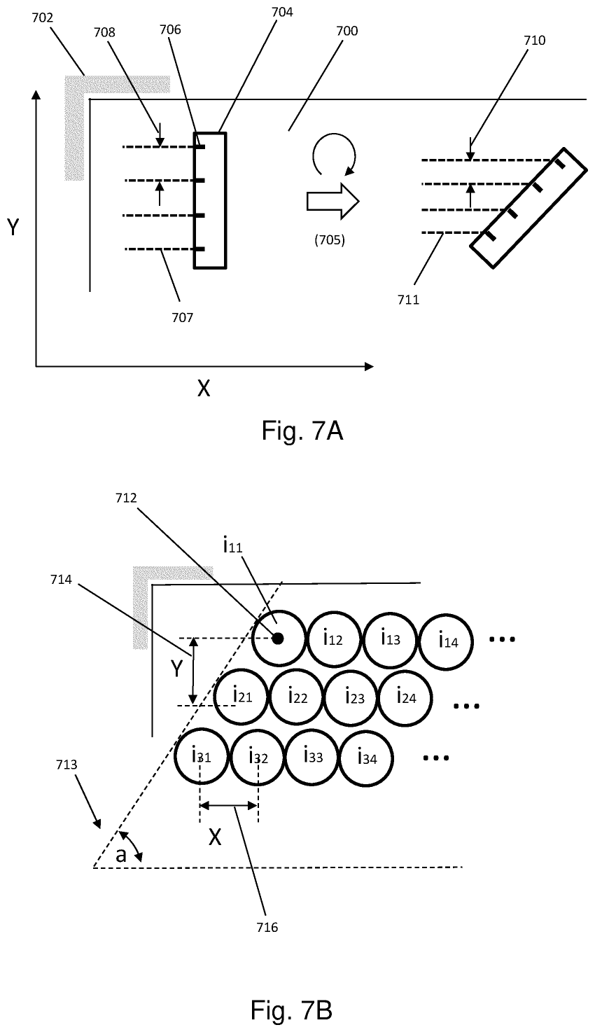

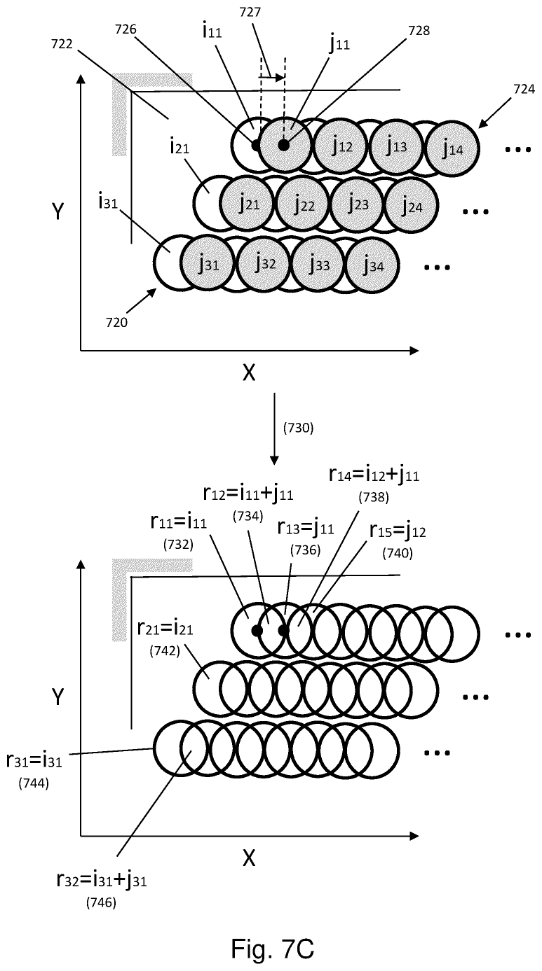

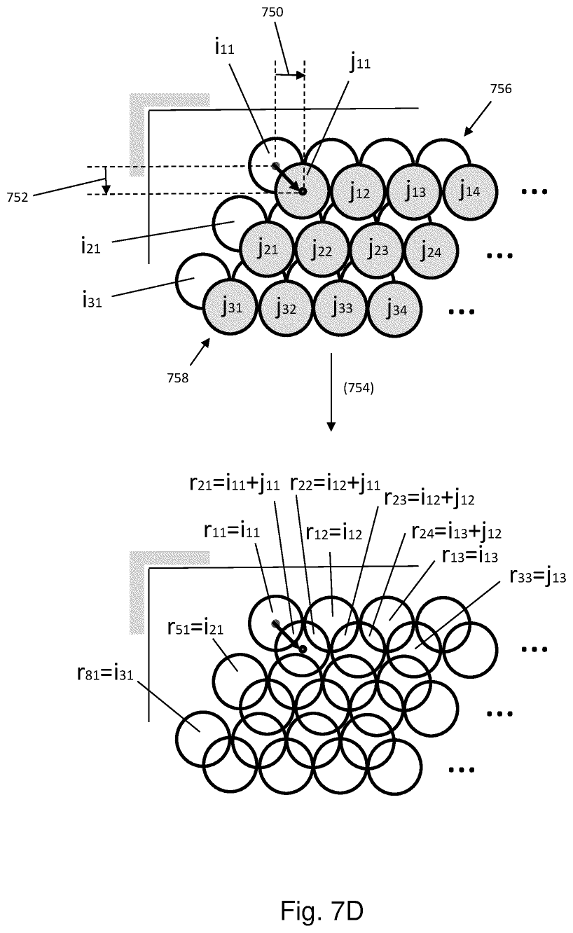

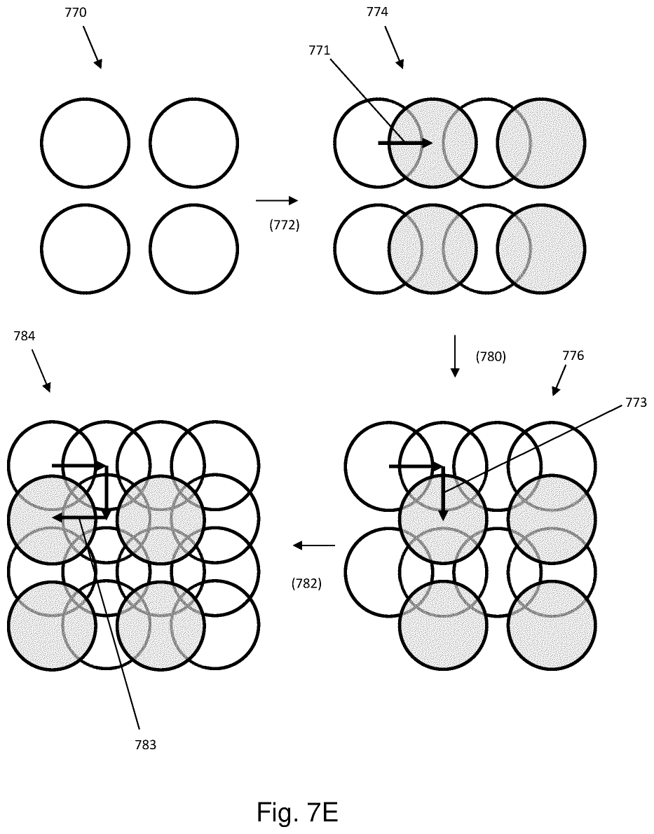

In typical inkjet synthesis applications, an array of distinct non-overlapping reaction sites is defined by repeated deposition of reagents. Usually, such reaction sites are roughly circular regions having diameters in the range of from about 20-50 μm. Thus, for some applications, such as those described in FIG. 5, the spatial resolution achievable by inkjet printed barcodes is very limited, especially if intracellular resolution is desired. In some embodiments, a higher density of unique barcodes may be synthesized on a surface by overwriting one array of oligonucleotides with another overlapping array of oligonucleotides. In other words, a first array of reaction sites may be defined on a surface on which a first set of oligonucleotides is synthesized, after which a second array of reaction sites is defined the same surface on which a second set of oligonucleotides is synthesized, such that the second array overlaps the first array so that the surface is partition into a larger number of smaller-sized regions in which there are unique barcodes. In some embodiments, for example, if the oligonucleotides of the first set are each m nucleotides in length with different predetermined, or known, sequences and the oligonucleotides of the second set are each n nucleotides in length, also with different predetermined, or known sequences, then there will be regions of the surface containing oligonucleotides that are m nucleotides in length, n nucleotides in length and n+m nucleotides in length, each with a known unique sequence in a known region. In other embodiments, oligonucleotides of selected reaction sites within the arrays may be capped to prevent further extensions. Exemplary embodiments are illustrated in FIGS. 7A-7E.

The formation of arrays of reaction sites with predetermined locations is illustrated in FIG. 7A for one type of print head. In some embodiments, substrate (700) may be physically positioned by angle stop (702) so that print head (704) of a inkjet printing instrument can be programmed to have a well-defined starting location for producing reaction sites of an array. Print head (704) is shown with four nozzles (706) with a predetermined “Y” axis spacing (708) dependent on the angle of print head (704) with respect to substrate (700) (for example, angle “a” with respect of the “X” axis, illustrated in FIG. 7B). In the left hand side of FIG. 7A, print head (704) is shown perpendicular to the “X” axis, so that rows of reaction sites (shown as dashed lines (707)) have maximum separation in the “Y” direction (shown as (714) in FIG. 1B). Separation of reaction sites in the “X” direction (e.g. (716) in FIG. 7B) is determined by (i) the predetermined frequency at which nozzles (706) generate and emit droplets and (ii) the speed at which print head (704) traverses substrate (700). The separation of reaction sites in the “Y” direction may be determined by rotating (705) print head (704) relative to the “X” and “Y” axes, as shown in the right hand side of FIG. 7A where rows (711) of reaction sites have closer spacing (710) than those of rows (707) of the left hand side of FIG. 7A. That is, a predetermined angle “a” ((713) in FIG. 7B) less than 90 degrees may be selected to determine inter-row distance (714) in the “Y” direction. This produces an array of reaction sites that is roughly parallelogram shaped and having origin, or reference, reaction site (712), as shown in FIG. 713. Oligonucleotides, i₁₁, i₁₂, . . . i₁ₛ, i₂₁, i₂₂, . . . i₂ₛ . . . iₖ₁, iₖ₂, . . . iₖₛ, may be synthesized in the reaction sites to produce an s by k array of barcodes, or barcode segments, which may or may not have distinct sequences from other barcode segments. Each of the reaction sites in the array has a predetermined position relative to the reference, or origin, reaction site (which is shown as the upper left-most reaction site in the figures for convenience, but could be any reaction site)

An example of how overwriting may increase the density of spatial barcoding is illustrated in FIG. 7C. In the upper portion, substrate (722) is shown with array (720) of oligonucleotides i₁₁, i₁₂, . . . (open circles) having origin, or reference position, (726). On top of array (720), array (724) of oligonucleotides j₁₁, j₁₂, . . . having origin, or reference position, (728) is synthesized (shown as filled circles). In some embodiments, arrays (720) and (724) have identical X and Y spacing and angle “a”. The only geometrical or spatial difference between the arrays is that the origin of array (724) is translated distance (727) in the X direction relative to array (720). This creates (730), as shown in the bottom portion of FIG. 7C, host of new regions on substrate (722) each with a barcode, which by judicious selection of sequences of oligonucleotides i₁₁, i₁₂, . . . , and oligonucleotides j₁₁, j₁₂, . . . may be unique. In this particular example, each row of four subregions of an area is transformed into a row of 15 subregions in the same area: end region (732) with oligonucleotide r₁₁=j₁₁, almond-shaped region (734) with oligonucleotide r₁₂=i₁₁+j₁₁, hourglass region (736) with oligonucleotide r₁₃=j₁₁, almond-shaped region (738) with oligonucleotide r₁₄=i₁₂+j₁₁, hourglass region (740) with oligonucleotide r₁₅=j₁₂, and so on. (The terminology “i₁₂+j₁₁”, and like terms, mean that a composite oligonuclcotide is synthesized. For example, if i₁₂ is 5′-AATCCG-3′ and j₁₁ is 5′-TTGGA-3′, then the oligonucleotide i₁₂+j₁₁, is “5′-AATCCGTTGGA-3′) In some embodiments, the lengths of oligonucleotide in such overlapping arrays are selected so that each region has a unique barcode. Thus, the lengths depend in part on how many reaction sites are present in the arrays. In some embodiments, the lengths of each oligonucleotide is in the range of between 2 and 12 nucleotides, or between 3 and 8 nucleotides. The lengths of oligonucleotides in successive arrays may be the same or different.

The translation of the positions of a subsequent array relative to a previous array may be carried out using two XY-stages, one to move the print head to generate an array of oligonucleotides, and another to provide the offset or new position of the reference reaction site relative to the reference reaction site of a previously synthesized army. The two XY stages may be used in tandem. e.g. one mounted on top of the other, or one may be used to move the print head, which the other moves the mounting stage holding the substrate. In other embodiments, a single XY stage that can be programmed to generate the desired offsets may be used. For example. Offsets can also be introduced by using high dpi print heads and choosing whether to switch on certain nozzles in the y direction that were not previously being used.

As illustrated in FIG. 7D, overlapping arrays need not be produced only by simple translations in the X direction or the Y direction alone. Overlapping arrays may be produced by translations in both the X direction (750) and the Y direction (752), where as above, first array (756) of reaction sites is illustrated as open circles and second array (758) of reaction sites is illustrated as shaded circles. As above, a complicated pattern of regions are produced (754) in which different barcodes are synthesized, which may be distinct and unique from every other barcode by judicious selection of the lengths and sequences of the i and j subunits.

As illustrated in FIG. 7E, in some embodiments a plurality of overwritings based on a plurality of translations of an array of reaction sites may be carried out. For example, a first array (770) is shown as a rectilinear array of four reaction sites each containing different oligonucleotides i₁₁, i₁₂, and so on. The origin of the array is shifted (772) a predetermined distance (771) in the X direction after which a second set of oligonucleotides i₁₁, i₂, . . . (shaded circles) is synthesized, to produce additional regions (774) similar to those illustrated in FIG. 7C. After such second synthesis, the origin of the array is again shifted (780) a predetermined distance (773) in the negative Y direction after which a third set of oligonucleotides k₁₁, k₁₂, . . . (shaded circles) is synthesized to produce pattern (776) of regions. The origin of the array may be shifted (782) a predetermined distance (783) in the negative X direction a third time, after which a fourth set of oligonucleotides l₁₁, l₁₂, . . . is synthesized. This produces pattern (784) of regions each capable of having a unique barcode, which may comprise a single i, j, k, or l segment, or may comprise a composite of up to all four such components.

In some embodiments, the invention is directed to a method of enzymatically synthesizing a plurality of oligonucleotide barcodes each having a predetermined sequence at distinct predetermined regions of substrate, such method comprising the steps of: (a) providing a substrate with a surface comprising a coating of initiators, wherein each initiator has a free 3-hydroxyl; (b) determining a position of a reference reaction site of an array of a plurality of reaction sites, the position of each reference reaction site after the first reference site position is selected so that at least one reaction site of the array overlaps a reaction site of the previous array; (c) synthesizing an oligonucleotide in each reaction site of the array by (i) dispensing through one or more inkjet pumps at least one droplet of at least one synthesis reagent to each reaction site of the plurality to perform a reaction cycle comprising the steps of (A) reacting under elongation conditions the initiator or elongated fragments having free 3′-O-hydroxyls with a 3′-O-protected nucleoside triphosphate and a template-free DNA polymerase so that the initiator or elongated fragments are elongated by incorporation of a 3′-O-protected nucleoside triphosphate to form 3′-O-protected elongated fragments, and (B) deprotecting the elongated fragments to form elongated fragments having free 3′-hydroxyls, wherein the synthesis reagent comprises a template-free polymerase, a 3′-O-protected nucleoside triphosphate, a mixture of a template-free polymerase and a 3′-O-protected nucleoside triphosphate, or a deprotection solution; and (ii) repeating a predetermined number of times step (i) until the plurality of oligonucleotides is synthesized; (c) repeating steps (b) and (c) until the plurality of oligonucleotide barcodes are synthesized each having a predetermined sequence at predetermined regions of the substrate comprising overlapping and non-overlapping reaction sites. In some embodiments, each array of step (b) is the same except for the location of its reference reaction site. In some embodiments, each array of step (b) has the same plurality of reaction sites and the same pattern and inter-reaction site distances. In some embodiments, arrays of reaction sites are identical rectilinear arrays (such as illustrated in FIG. 1B) and successive arrays in the above method are formed by printing an identical copy of the previous array, except for the movement, or shifting, of the new array's reference reaction site a predetermined distance from that of the previous array, e.g. as illustrated in FIGS. 7C and 7D for the arrays having parallelogram shapes. In some embodiments, the predetermined distance is non-zero and equal to or less than the average distance between centers of adjacent reaction sites of the array. In some embodiments, the predetermined distance is in the range of from one tenth to twice the average distance between centers of adjacent reaction sites of the array. In some embodiments, the predetermined distance is solely along one axis defining the array, such as illustrated in FIG. 7C. In some embodiments, overlapping arrays may be formed by rotating subsequent arrays, e.g. about the center of a reference reaction site, relative to a previous array. In some embodiments, steps (b) and (c) are repeated a number of times in the range of from 1 to 4, or from 1 to 3, or from 1 to 2, or steps (b) and (c) are repeated once.

### Methods of Template-Free Enzymatic Synthesis

Generally, methods of template-free (or equivalently. “template-independent”) enzymatic polynucleotide synthesis comprise repeated cycles of steps, such as are illustrated in FIG. 1A, in which a predetermined nucleotide is coupled to an initiator or growing chain in each cycle. The general elements of template-free enzymatic synthesis are described in the following references: Ybert et al, International patent publication WO/2015/159023; Ybert et al, International patent publication WO/2017/216472; Hyman, U.S. Pat. No. 5,436,143; Hiatt et al, U.S. Pat. No. 5,763,594; Jensen et al, Biochemistry, 57: 1821-1832 (2018); Mathews et al. Organic & Biomolecular Chemistry. DOI: 0.1039/c6ob01371f (2016); Schmitz et al. Organic Lett., 1(11): 1729-1731 (1999).

In the present invention synthesis reagents delivered by inkjet pumps must be formulated to satisfy at least two constraints: (i) the need to preserve the elongation activity of the template-free polymerase (in the case of template-free polymerase inks), and (ii) the need to meet the rheological requirements for droplet formation. The key solution parameters affecting droplet formation by inkjets are viscosity, surface tension, liquid density and the diameter of the inkjet nozzle. For particular embodiments of the invention, synthesis reagents prepared for non-droplet delivery to a reaction mixture may be reformulated by adding viscosity modifiers, surface tension modifiers and density modifiers, and the like, in order to form “printable inks” that may be delivered in droplets generated by inkjet pumps. “Printable” in reference to a reagent ink means repeatable droplets are able to be ejected from the nozzle, with uniform velocities and volumes and without satellite droplets.

As illustrated in FIG. 1A, initiator polynucleotides (100) with free 3′-hydroxyl groups (130) are provided, for example, attached to synthesis support (120). To the initiator polynucleotides (100) (or elongated initiator polynucleotides in subsequent cycles) are added a 3′-O-protected-dNTP and a template-free polymerase, such as a terminal deoxynucleotidyltransferase (TdT) or variant thereof (e.g. Ybert et al. WO/2017/216472; Champion et al. WO2019/135007) under conditions (140) effective for the enzymatic incorporation of the 3′-O-protected-dNTP onto the 3′ end of the initiator polynucleotides (100) (or elongated initiator polynucleotides). This reaction produces elongated initiator polynucleotides whose 3′-hydroxyls are protected (160). If the elongated sequence is not complete, then another cycle of addition is implemented (180). If the elongated initiator polynucleotide contains a competed sequence, then the 3′-O-protection group may be removed, or deprotected, and the desired sequence may be cleaved from the original initiator polynucleotide (182). Such cleavage may be carried out using any of a variety of single strand cleavage techniques, for example, by inserting a cleavable nucleotide at a predetermined location within the original initiator polynucleotide. An exemplary cleavable nucleotide may be a uracil nucleotide which is cleaved by uracil DNA glycosylase. In accordance with some embodiments of the invention, cleavage reagents may be delivered to reaction sites in droplets generated by inkjet pumps. In such embodiments, polynucleotides at reaction sites known to be incomplete or otherwise defective can be separated from fully competed polynucleotides or can be selectively re-synthesized either by cleaving and re-synthesizing the entire polynucleotide, or by cleaving or otherwise removing incorrect sequences and re-synthesizing only the defective part of the polynucleotide.

If the elongated initiator polynucleotide is not a completed sequence (i.e. the end product), then the 3′-O-protection groups are removed to expose free 3′-hydroxyls (130) and the elongated initiator polynucleotides are subjected to another cycle of nucleotide addition and deprotection.

As used herein, an “initiator” (or equivalent terms, such as “initiating fragment,” “initiator nucleic acid.” “initiator oligonucleotide.” or the like) usually refers to a short oligonucleotide sequence with a free 3′-hydroxyl at its end, which can be further elongated by a template-free polymerase, such as TdT. In one embodiment, the initiating fragment is a DNA initiating fragment. In an alternative embodiment, the initiating fragment is an RNA initiating fragment. In some embodiments, an initiating fragment possesses between 3 and 100 nucleotides, in particular between 3 and 20 nucleotides. In some embodiments, the initiating fragment is single-stranded. In alternative embodiments, the initiating fragment may be double-stranded. In some embodiments, an initiator oligonucleotide may be attached to a synthesis support by its 5′end; and in other embodiments, an initiator oligonucleotide may be attached indirectly to a synthesis support by forming a duplex with a complementary oligonucleotide that is directly attached to the synthesis support, e.g. through a covalent bond. In some embodiments a synthesis support is a solid support which may be a discrete region of a solid planar solid, or may be a bead.

In some embodiments, an initiator may comprise a non-nucleic acid compound having a free hydroxyl to which a TdT may couple a 3′-O-protected dNTP, e.g. Baiga. U.S. patent publications US201910078065 and US2019/0078126.

After synthesis is completed polynucleotides with the desired nucleotide sequence may be released from initiators and the synthesis supports by cleavage.

A wide variety of cleavable linkages or cleavable nucleotides may be used for this purpose. In some embodiments, cleaving the desired polynucleotide leaves a natural free 5′-hydroxyl on a cleaved strand; however, in alternative embodiments, a cleaving step may leave a moiety, e.g. a 5′-phosphate, that may be removed in a subsequent step, e.g. by phosphatase treatment. Cleaving steps may be carried out chemically, thermally, enzymatically or by photochemical methods. In some embodiments, cleavable nucleotides may be nucleotide analogs such as deoxyuridine or 8-oxo-deoxyguanosine that are recognized by specific glycosylases (e.g. uracil deoxyglycosylase followed by endonuclease VIII, and 8-oxoguanine DNA glycosylase, respectively). In some embodiments, cleavage may be accomplished by providing initiators with a deoxyinosine as the penultimate 3′ nucleotide, which may be cleaved by endonuclease V at the 3′ end of the initiator leaving a 5′-phosphate on the released polynucleotide. In some embodiments, an initiator may contain a terminal uridine so that after synthesis the desired polynucleotide may be cleaved from the initiator by treatment with KOH, or like base. Further methods for cleaving single stranded polynucleotides are disclosed in the following references, which are incorporated by reference: U.S. Pat. Nos. 5,739,386, 5,700,642 and 5,830,655; and U.S. Patent Publication Nos. 2003/0186226 and 2004/0106728; and in Urdea and Horn, U.S. Pat. No. 5,367,066.

Returning to FIG. 1A, in some embodiments, an ordered sequence of nucleotides are coupled to an initiator nucleic acid using a template-free polymerase, such as TdT, in the presence of 3′-O-protected dNTPs in each synthesis step. In some embodiments, the method of synthesizing an oligonucleotide comprises the steps of (a) providing an initiator having a free 3′-hydroxyl; (b) reacting under extension (or elongation) conditions the initiator or an extension intermediate having a free 3′-hydroxyl with a template-free polymerase in the presence of a 3′-O-protected nucleoside triphosphate to produce a 3′-O-protected extension intermediate; (c) deprotecting the extension intermediate to produce an extension intermediate with a free 3′-hydroxyl; and (d) repeating steps (b) and (c) until the polynucleotide is synthesized. (Sometimes the terms “extension intermediate” or “elongation fragment” or “growing chain” are used interchangeably). As used herein, the term “elongation conditions” means physical and chemical conditions of a reaction mixture necessary for a template-free polymerase to catalyze an elongation reaction wherein a 3′-O-protected nucleoside triphosphate monomer is coupled (by formation of a phosphodiester bond) to a free 3′-hydroxy of a nucleic acid fragment which, for example, may be an initiator or an elongated fragment. Exemplary elongation conditions include selections of reaction temperature, reaction duration. pH, concentrations of various salts, scavengers of undesired reaction components, agents to reduce nucleic acid secondary structures, and the like. In some embodiments, an initiator is provided as an oligonucleotide attached to a solid support. e.g. by its 5′ end. The above method may also include washing steps after the reaction, or extension, step, as well as after the de-protecting step. For example, the step of reacting may include a sub-step of removing unincorporated nucleoside triphosphates. e.g. by washing, after a predetermined incubation period, or reaction time. In some embodiments, such predetermined incubation periods or reaction times may be in the range of from 30 seconds to 30 minutes, or from 1 min to 30 min, or from 1 min to 15 min, or from 1 min to 10 min, or from 30 sec to 5 min.

In some embodiments, after the synthesis cycles of FIG. 1A are completed further steps may be performed to cleave the completed polynucleotides from the solid supports. Such further steps may be performed at the reaction sites of the array. Additionally, some cleavage methods may result in a released product that still requires modification to convert it into a useable product. For example, in the “endonuclease V-inosine” cleavage (described below) leaves a 5′-phosphate that must be removed for some applications. Thus, a further step of phosphatase treatment may be required.

When the predetermined sequences of polynucleotides on a synthesis support includes reverse complementary subsequences, secondary intra-molecular or cross-molecular structures may be created by the formation of hydrogen bonds between the reverse complementary regions. In some embodiments, base protecting moieties for exocyclic amines are selected so that hydrogens of the protected nitrogens cannot participate in hydrogen bonding, thereby preventing the formation of such secondary structures. That is, base protecting moieties may be employed to prevent the formation of hydrogen bonds, such as are formed in normal base pairing, for example, between nucleosides A and T and between G and C. At the end of a synthesis, the base protecting moieties may be removed and the polynucleotide product may be cleaved from the solid support, for example, by cleaving it from its initiator.

In addition to providing 3′-O-blocked dNTP monomers with base protection groups, elongation reactions may be performed at higher temperatures using thermal stable template-free polymerases. For example, a thermal stable template-free polymerase having activity above 40° C. may be employed; or, in some embodiments, a thermal stable template-free polymerase having activity in the range of from 40-85° C. may be employed; or, in some embodiments, a thermal stable template-free polymerase having activity in the range of from 40-65° C. may be employed.

In some embodiments, elongation conditions may include adding solvents to an elongation reaction mixture that inhibit hydrogen bonding or base stacking. Such solvents include water miscible solvents with low dielectric constants, such as dimethyl sulfoxide (DMSO), methanol, and the like. Likewise, in some embodiments, elongation conditions may include the provision of chaotropic agents that include, but are not limited to, n-butanol, ethanol, guanidinium chloride, lithium perchlorate, lithium acetate, magnesium chloride, phenol, 2-propanol, sodium dodecyl sulfate, thiourea, urea, and the like. In some embodiments, elongation conditions include the presence of a secondary-structure-suppressing amount of DMSO. In some embodiments, elongation conditions may include the provision of DNA binding proteins that inhibit the formation of secondary structures, wherein such proteins include, but are not limited to, single-stranded binding proteins, helicases. DNA glycolases, and the like.

When base-protected dNTPs are employed, the above method of FIG. 1A may further include a step (c) removing base protecting moieties, which in the case of acyl or amidine protection groups may (for example) include treating with concentrated ammonia.

The above method may also include capping step(s) as well as washing steps after the reacting, or extending, step, as well as after the deprotecting step. As mentioned above, in some embodiments, capping steps may be included in which non-extended free 3′-hydroxyls are reacted with compounds that prevents any further extensions of the capped strand. In some embodiments, such compound may be a dideoxynucleoside triphosphate. In other embodiments, non-extended strands with free 3′-hydroxyls may be degraded by treating them with a 3′-exonuclease activity, e.g. Exo I. For example, see Hyman, U.S. Pat. No. 5,436,143. Likewise, in some embodiments, strands that fail to be deblocked may be treated to either remove the strand or render it inert to further extensions. When a capping agent, such as ddNTPs, are used, the buffer or synthesis reagents containing such agents may be delivered by flowing or spraying such reagent over substrate containing the reaction sites.

In some embodiments, reaction conditions for an elongation step (also sometimes referred to as an extension step or a coupling step) may comprising the following: 2.0 μM purified TdT; 125-600 μM 3′-O-blocked dNTP (e.g. 3′-O—NH₂-blocked dNTP); about 10 to about 500 mM potassium cacodylate buffer (pH between 6.5 and 7.5) and from about 0.01 to about 10 mM of a divalent cation (e.g. CoCl₂ or MnCl₂), where the elongation reaction may be carried out at a temperature within the range RT to 45° C., for 3 minutes. It is understood that whenever the foregoing coupling reagent is delivered by inkjet-produced droplets its viscosity, density and surface tension must be adjusted so that it becomes a printable ink. In this connection, the invention in part includes the recognition and appreciation that an ink for delivering TdT to a reaction site may have its viscosity modified for droplet formation and activity preserved by selection of a viscosity modifier, such as, when carboxymethyl cellulose is selected as the viscosity modifying agent.

In embodiments, in which the 3′-O-blocked dNTPs are 3′-O—NH₂-blocked dNTPs, reaction conditions for a deblocking step may comprise the following: 700 mM NaNO₂; 1 M sodium acetate (adjusted with acetic acid to pH in the range of 4.8-6.5), where the deblocking reaction may be carried out at a temperature within the range of RT to 45° C. for 30 seconds to several minutes. Washes may be performed with the cacodylate buffer without the components of the coupling reaction (e.g. enzyme, monomer, divalent cations). If the above reagent compositions are delivered to reaction sites by inkjet delivery, it is understood that the compositions would be altered to meet the rheological requirements for droplet formation by the nozzles of the inkjet print heads used.

In some embodiments, RNA synthesis may be accomplished by similar steps as described above but with template-free polymerases and monomers specifically selected for RNA synthesis, such as, polyA polymerase (PAP), polyU polymerase (PUP), or the like, e.g. International patent publication WO2020/077227. For example, systems, apparatus and kits of the invention may implement methods of synthesizing a polyribonucleotide having a predetermined sequence comprising the steps of: a) providing an initiator having a 3′-terminal nucleotide having a free 3′-hydroxyl; and b) repeating, until the polyribonucleotide is formed, cycles of (i) contacting under elongation conditions the initiator or elongated fragments having free 3′-hydroxyls with a 3′-O-blocked-nucleoside triphosphate and a template-free polymerase so that the initiator or elongated fragments are elongated by incorporation of a 3′-O-blocked-nucleoside triphosphate to form 3′-O-blocked-elongated fragments, and (ii) deblocking the elongated fragments to form elongated fragments having free 3′-hydroxyls; wherein the template-free polymerase is a poly(A) polymerase (PAP) or a poly(U) polymerase. In further embodiments, the initiator may be attached to a support by a 5′ end, the support may be a solid support, and the above method may include a step of cleaving the polynucleotide from the initiator. In some embodiments, reaction conditions for an extension or elongation step using PAP or PUP may comprising the following: Reaction conditions 1 (for primer+AM-rATP): 250 uM AM-rATP, 0.1 uM ATTO488-(rA)5, 1 uM PAP, 1×ATP buffer (20 mM Tris-HCl, 0.6 mM MnCl2, 0.02 mM EDTA, 0.1% BSA, 10% glycerol, 100 mM imidazole, pH 7-8), 37 C, 30 min. Reaction condition 2 (for primer+AM-rGTP): 250 uM rGTP, 0.1 uM ATT0488-(rA)5, 1 uM PAP, 1×GTP buffer (0.6 mM MnCl2, 0.1% BSA, 10 mM imidazole, pH 6), 37 C, 30 min. In the foregoing, “AM-rNTP” refers to 3′-azidomethyl-O-ribonucleoside triphosphate. Many of the 3′-O-blocked rNTPs employed in the invention may be purchased from commercial vendors (e.g. Jena Bioscience, MyChemlabs, or the like) or synthesized using published techniques, e.g. U.S. Pat. No. 7,057,026; International patent publications WO2004/005667, WO91/06678; Canard et al. Gene (cited above); Metzker et al, Nucleic Acids Research, 22: 4259-4267 (1994); Meng et al, J. Org. Chem., 14: 3248-3252 (3006): U.S. patent publication 2005/037991; Zavgorodny et al. Tetrahedron Letters, 32(51): 7593-7596 (1991). In a further particular embodiments, the 3′-blocked nucleotide triphosphate is blocked by either 3′-O-propargyl, a 3′-O-azidomethyl, 3′-O—NH₂ or 3′-O-allyl group. In still other embodiments, 3′-O-blocking groups of the invention include 3′-O-methyl, 3′-O-(2-nitrobenzyl)#3′-O-allyl, 3′-O-amine, 3′-O-azidomethyl, 3′-O-tert-butoxy ethoxy, 3′-O-(2-cyanoethyl), and 3′-O-propargyl. As above, if the above reagent compositions are delivered to reaction sites by inkjet delivery, it is understood that the compositions would be altered to meet the rheological requirements for droplet formation by the nozzles of the inkjet print heads used.

### 3′-O-Protected Nucleoside Triphosphates

Depending on particular applications, the steps of deblocking and/or cleaving may include a variety of chemical or physical conditions. e.g. light, heat. pH, presence of specific reagents, such as enzymes, which are able to cleave a specified chemical bond. Guidance in selecting 3′-O-blocking groups and corresponding de-blocking conditions may be found in the following references, which are incorporated by reference: Benner, U.S. Pat. Nos. 7,544,794 and 8,212,020; 5,808,045; 8,808,988; International patent publication WO91/06678; and references cited below. In some embodiments, the cleaving agent (also sometimes referred to as a de-blocking reagent or agent) is a chemical cleaving agent, such as, for example, dithiothreitol (DTT). In alternative embodiments, a cleaving agent may be an enzymatic cleaving agent, such as, for example, a phosphatase, which may cleave a 3′-phosphate blocking group. It will be understood by the person skilled in the art that the selection of deblocking agent depends on the type of 3′-nucleotide blocking group used, whether one or multiple blocking groups are being used, whether initiators are attached to living cells or organisms or to solid supports, and the like, that necessitate mild treatment. For example, a phosphine, such as tris(2-carboxyethyl)phosphine (TCEP) can be used to cleave a 3′O-azidomethyl groups, palladium complexes can be used to cleave a 3′O-allyl groups, or sodium nitrite can be used to cleave a 3′O-amino group. In particular embodiments, the cleaving reaction involves TCEP, a palladium complex or sodium nitrite.

As noted above, in some embodiments it is desirable to employ two or more blocking groups that may be removed using orthogonal de-blocking conditions. The following exemplary pairs of blocking groups may be used in parallel synthesis embodiments. It is understood that other blocking group pairs, or groups containing more than two, may be available for use in these embodiments of the invention.

| | |
|---|---|
| 3′-O—NH2 | 3′-O-azidomethyl |
| 3′-O—NH2 | 3′-O-allyl, 3′-O-propargyl |
| 3′-O—NH2 | 3′-O-phosphate |
| 3′-O-azidomethyl | 3′-O-ally1, 3′-O-propargyl |
| 3′-O-azidometbyl | 3′-O-phosphate |
| 3′-O-allyl, 3′-O-propargyl | 3′-O-phosphate |

In some embodiments, specific enzymatically removable blocking groups are require specific enzymes for their removal. For example, ester- or acyl-based blocking groups may be removed with an esterase, such as acetylesterase, or like enzyme, and a phosphate blocking group may be removed with a 3′ phosphatase, such as T4 polynucleotide kinase. By way of example, 3′-O-phosphates may be removed by treatment with as solution of 100 mM Tris-HCl (pH 6.5) 10 mM MgCl₂, 5 mM 2-mercaptoethanol, and one Unit T4 polynucleotide kinase. The reaction proceeds for one minute at a temperature of 37° C. As above, if the foregoing compositions are delivered to reaction sites by inkjet delivery, it is understood that the compositions would be altered to meet the rheological requirements for droplet formation by the nozzles of the inkjet print heads used.

Further examples of synthesis and enzymatic deprotection of 3′-O-ester-protected dNTPs or 3′-O-phosphate-protected dNTPs are described in the following references: Canard et al. Proc. Natl. Acad. Sci., 92:10859-10863 (1995); Canard et al, Gene, 148: 1-6 (1994); Cameron et al, Biochemistry, 16(23): 5120-5126 (1977); Rasolonjatovo et al, Nucleosides & Nucleotides, 18(4&5): 1021-1022 (1999): Ferrero et al. Monatshefte fur Chemie, 131: 585-616 (2000); Taunton-Rigby et al, J. Org. Chem., 38(5): 977-985 (1973): Uemura et al, Tetrahedron Lett., 30(29): 3819-3820 (1989); Becker et al, J. Biol. Chem., 242(5): 936-950 (1967); Tsien, International patent publication WO1991/006678.

In some embodiments, the modified nucleotides comprise a modified nucleotide or nucleoside molecule comprising a purine or pyrimidine base and a ribose or deoxyribose sugar moiety having a removable 3′-OH blocking group covalently attached thereto, such that the 3′ carbon atom has attached a group of the structure:

—O—Z

wherein —Z is any of —C(R′)₂—O—R″, —C(R′)₂—N(R′)₂, —C(R′)₂—N(H)R″, —C(R′)₂—S—R″ and —C(R′)₂—F, wherein each R″ is or is part of a removable protecting group; each R′ is independently a hydrogen atom, an alkyl, substituted alkyl, arylalkyl, alkenyl, alkynyl, aryl, heteroaryl, heterocyclic, acyl, cyano, alkoxy, aryloxy, heteroaryloxy or amido group, or a detectable label attached through a linking group; with the proviso that in some embodiments such substituents have up to 10 carbon atoms and/or up to 5 oxygen or nitrogen heteroatoms; or (R′)₂ represents a group of formula ═(R′″)₂ wherein each R′″ may be the same or different and is selected from the group comprising hydrogen and halogen atoms and alkyl groups, with the proviso that in some embodiments the alkyl of each R′″ has from 1 to 3 carbon atoms; and wherein the molecule may be reacted to yield an intermediate in which each R″ is exchanged for H or, where Z is —(R′)₂—F, the F is exchanged for OH, SH or NH₂, preferably OH, which intermediate dissociates under aqueous conditions to afford a molecule with a free 3′-OH; with the proviso that where Z is —C(R′)₂—S—R″, both R′ groups are not H. In certain embodiments. R′ of the modified nucleotide or nucleoside is an alkyl or substituted alkyl, with the proviso that such alkyl or substituted alkyl has from 1 to 10 carbon atoms and from 0 to 4 oxygen or nitrogen heteroatoms. In certain embodiments, −Z of the modified nucleotide or nucleoside is of formula —C(R′)₂-N3. In certain embodiments, Z is an azidomethyl group.

In some embodiments. Z is a cleavable organic moiety with or without heteroatoms having a molecular weight of 200 or less. In other embodiments. Z is a cleavable organic moiety with or without heteroatoms having a molecular weight of 100 or less. In other embodiments. Z is a cleavable organic moiety with or without heteroatoms having a molecular weight of 50 or less. In some embodiments, Z is an enzymatically cleavable organic moiety with or without heteroatoms having a molecular weight of 200 or less. In other embodiments, Z is an enzymatically cleavable organic moiety with or without heteroatoms having a molecular weight of 100 or less. In other embodiments, Z is an enzymatically cleavable organic moiety with or without heteroatoms having a molecular weight of 50 or less. In other embodiments. Z is an enzymatically cleavable ester group having a molecular weight of 200 or less. In other embodiments. Z is a phosphate group removable by a 3′-phosphatase. In some embodiments, one or more of the following 3-phosphatases may be used with the manufacturer's recommended protocols: T4 polynucleotide kinase, calf intestinal alkaline phosphatase, recombinant shrimp alkaline phosphatase (e.g. available from New England Biolabs, Beverly, MA)

In a further embodiments, the 3′-blocked nucleotide triphosphate is blocked by either a 3′-O-azidomethyl, 3′-O—NH₂ or 3′-O-allyl group.

In still other embodiments, 3′-O-blocking groups of the invention include 3′-O-methyl, 3′-O-(2-nitrobenzyl), 3′-O-allyl, 3′-O-amine, 3′-O-azidomethyl, 3′-O-tert-butoxy ethoxy, 3′-O-(2-cyanoethyl), and 3′-O-propargyl.

3′-O-blocked dNTPs without base protection may be purchased from commercial vendors or synthesized using published techniques. e.g. U.S. Pat. No. 7,057,026; Guo et al. Proc. Natl. Acad. Sci., 105(27): 9145-9150 (2008); Benner. U.S. Pat. Nos. 7,544,794 and 8,212,020; International patent publications WO2004/005667, WO91/06678; Canard et al. Gene (cited herein); Metzker ct al, Nucleic Acids Research, 22: 4259-4267 (1994); Meng et al, J. Org. Chem., 14: 3248-3252 (2006); U.S. patent publication 2005/037991. 3′-O-blocked dNTPs with base protection may be synthesized as described below.

### Template-Free Polymerases for Polynucleotide Synthesis

A variety of different template-free polymerases are available for use in methods of the invention. Template-free polymerases include, but are not limited to, polX family polymerases (including DNA polymerases β, λ and μ), poly(A) polymerases (PAPs), poly(U) polymerases (PUPs), DNA polymerase θ, and the like, for example, described in the following references: Ybert et al. international patent publication WO2017/216472; Champion et al, U.S. patent Ser. No. 10/435,676; Champion et al, International patent publication WO2020/099451; Heinisch et al. International patent publication WO2021/018919. In particular, terminal deoxynucleotidyltransferases (TdTs) and variants thereof are useful in template-free DNA synthesis.

In some embodiments, TdT variants are employed with the invention which display increased incorporation activity with respect to 3′-O-amino nucleoside triphosphates. For example, such TdT variants may be produced using techniques described in Champion et al. U.S. patent Ser. No. 10/435,676, which is incorporated herein by reference. In some embodiments, a TdT variant is employed having (a) an amino acid sequence at least 80 percent identical to a TdT having an amino acid sequence of any of SEQ ID NOs 7 through 20, inclusive, and 24 through 39, inclusive, and (b) one or more of the substitutions listed in Table 1, wherein the TdT variant (i) is capable of synthesizing a nucleic acid fragment without a template and (ii) is capable of incorporating a 3′-O-modified nucleotide onto a free 3′-hydroxyl of a nucleic acid fragment. In some embodiments, the above TdT variants include a substitution at every position listed in Table 1. In some embodiments, the above percent identity value is at least 85 percent identity with the indicated SEQ ID NOs; in some embodiments, the above percent identity value is at least 90 percent identity with the indicated SEQ ID) NOs; in some embodiments, the above percent identity value is at least 95 percent identity with the indicated SEQ ID NOs; in some embodiments, the above percent identity value is at least 97 percent identity; in some embodiments, the above percent identity value is at least 98 percent identity; in some embodiments, the above percent identity value is at least 99 percent identity. As used herein, the percent identity values used to compare a reference sequence to a variant sequence do not include the expressly specified amino acid positions containing substitutions of the variant sequence, that is, the percent identity relationship is between sequences of a reference protein and sequences of a variant protein outside of the expressly specified positions containing substitutions in the variant.

TABLE 1

| SEQ | | | | | | |
|---|---|---|---|---|---|---|
| ID | | | | | | |
| NO | Animal | Substitutions | | | | |
| 1 | Mouse | M192R/Q | C302G/R | R336L/N | R454P/N/A/V | E457N/L/T/S/K |
| 7 | Mouse | M63R/Q | C173G/R | R207L/N | R325P/N/A/V | E328N/L/T/S/K |
| 8 | Bovine | M63R/O | C173G/R | R207L/N | R324P/N/A/V | E327N/L/T/S/K |
| 9 | Human | M63R/Q | C173G/R | R207L/N | R324P/N/A/V | E327N/L/T/S/K |
| 10 | Chicken | — | C172G/R | R206L/N | R320P/N/A/V | — |
| 11 | Possum | M63R/Q | C173G/R | R207L/N | R331P/N/A/V | E334N/L/T/S/K |
| 12 | Shrew | M63R/Q | C173G/R | R207L/N | — | E328N/L/T/S/K |
| 13 | Python | — | C174G/R | R208L/N | R331P/N/A/V | E334N/L/T/S/K |
| 14 | Canine | M73R/Q | C173G/R | R207L/N | R325P/N/A/V | E328N/L/T/S/K |
| 15 | Mole | M64R/Q | C174G/R | R208L/N | — | E329N/L/T/S/K |
| 16 | Pika | M61R/Q | C171G/R | R205L/N | R323P/N/A/V | E326N/L/T/S/K |
| 17 | Hedgehog | M63R/Q | C173G/R | R207L/N | R328P/N/A/V | E331N/L/T/S/K |
| 18 | Tree shrew | — | C173G/R | R207L/N | R325P/N/A/V | E328N/L/T/S/K |
| 19 | Platypus | M63R/Q | C182G/R | R216L/N | R338P/N/A/V | E341N/L/T/S/K |
| 20 | Jerboa | M66R/Q | C176G/R | R210L/N | R328P/N/A/V | E331N/L/T/S/K |
| 24 | Canary | — | C170G/R | R204L/N | R326P/N/A/V | E329N/L/T/S/K |
| 25 | Neopelma | — | C158G/R | R192L/N | R314P/N/A/V | E317N/L/T/S/K |
| 26 | Alligator | — | — | R205L/N | R327P/N/A/V | E330N/L/T/S/K |
| 27 | Xenopus | — | — | R205L/N | R324P/N/A/V | E327N/L/T/S/K |
| 28 | Tiger snake | — | — | R205L/N | R327P/N/A/V | E330N/L/T/S/K |
| 29 | Brown trout | — | — | R192L/N | R311P/N/A/V | E314N/L/T/S/K |
| 30 | Electric eel | — | — | R205L/N | R321P/N/A/V | E325N/L/T/S/K |
| 31 | Walking fish | — | — | R205L/N | R322P/N/A/V | E325N/L/T/S/K |
| 32 | Guppy | — | — | R205L/N | R322P/N/A/V | E325N/L/T/S/K |
| 33 | Rat | M48R/Q | C158G/R | R192L/N | R310P/N/A/V | E313N/L/T/S/K |
| 34 | Rat | M61R/Q | C171G/R | R205L/N | R323P/N/A/V | E326N/L/T/S/K |
| 35 | Colobus monkey | M61R/Q | C171G/R | R205L/N | R323P/N/A/V | E326N/L/T/S/K |
| 36 | Pig | M61R/Q | C171G/R | R205L/N | R323P/N/A/V | E326N/L/T/S/K |
| 37 | Tiger | M61R/Q | C171G/R | R205L/N | R323P/N/A/V | E326N/L/T/S/K |
| 38 | Water buffalo | M48R/Q | C158G/R | R192L/N | R310P/N/A/V | E313N/L/T/S/K |
| 39 | Marmot | M61R/Q | C171G/R | R205L/N | R323P/N/A/V | E326N/L/T/S/K |

In some embodiments, a TdT variant of the invention is derived from a TdT comprising an amino acid sequence at least 80 percent identical to an amino acid sequence selected from SEQ ID NOs 40 through 75, inclusive, and one or more of the substitutions listed in Table 2, wherein the TdT variant (i) is capable of synthesizing a nucleic acid fragment without a template and (ii) is capable of incorporating a 3′-O-modified nucleotide onto a free 3′-hydroxyl of a nucleic acid fragment. In some embodiments, the above TdT variants include a substitution at every position listed in Table 2. In some embodiments, the above percent identity value is at least 85 percent identity with the indicated SEQ ID NOs; in some embodiments, the above percent identity value is at least 90 percent identity with the indicated SEQ ID NOs; in some embodiments, the above percent identity value is at least 95 percent identity with the indicated SEQ ID NOs; in some embodiments, the above percent identity value is at least 97 percent identity; in some embodiments, the above percent identity value is at least 98 percent identity; in some embodiments, the above percent identity value is at least 99 percent identity. As above, the percent identity values used to compare a reference sequence to a variant sequence do not include the expressly specified amino acid positions containing substitutions of the variant sequence; that is, the percent identity relationship is between sequences of a reference protein and sequences of a variant protein outside of the expressly specified positions containing substitutions in the variant.

TdT variants of SEQ ID NOs 40 through 54, inclusive, 56, 59, 61, 63, 65, 67, 69, 70, 73 and 74 includes substitutions at one or more of the indicated amino acid positions as listed in Table 2 in addition to a stabilizing substitution of the glutamine at position 4 (or a functionally equivalent position). In other embodiments, TdT variants of the invention are derived from natural TdTs such as those listed in Table 2 with a substitution at every one of the indicated amino acid positions in addition to the stabilizing substitution of the glutamine at position 4. In some embodiments, such stabilizing amino acid substituted for glutamine is selected from the group consisting of E, S. D and N. In other embodiments, the stabilizing amino acid is E.

TABLE 2

| SEQ | | | | | | |
|---|---|---|---|---|---|---|
| ID | | | | | | |
| NO | Animal | Substitutions | | | | |
| 1 | Mouse | M192R/Q | C302G/R | R336L/N | R454P/N/A/V | E457N/L/T/S/K |
| 40 | Mouse | M44R/Q | C154G/R | R188L/N | R306P/N/A/V | E309N/L/T/S/K |
| 41 | Bovine | M44R/Q | C154G/R | R188L/N | R305P/N/A/V | E308N/L/T/S/K |
| 42 | Human | M44R/Q | C154G/R | R188L/N | R305P/N/A/V | E308N/L/T/S/K |
| 43 | Chicken | — | C154G/R | R188L/N | R302P/N/A/V | — |
| 44 | Possum | M44R/Q | C154G/R | R188L/N | R312P/N/A/V | E315N/L/T/S/K |
| 45 | Shrew | M44R/Q | C154G/R | R188L/N | — | E309N/L/T/S/K |
| 46 | Canine | M44R/Q | C154G/R | R188L/N | R306P/N/A/V | E309N/L/T/S/K |
| 47 | Mole | M44R/Q | C154G/R | R188L/N | — | E309N/L/T/S/K |
| 48 | Pika | M44R/Q | C154G/R | R188L/N | R306P/N/A/V | E309N/L/T/S/K |
| 49 | Hedgehog | M44R/Q | C154G/R | R188L/N | R309P/N/A/V | E312N/L/T/S/K |
| 50 | Tree shrew | — | C154G/R | R188L/N | R306P/N/A/V | E309N/L/T/S/K |
| 51 | Platypus | M44R/Q | C163G/R | R197L/N | R319P/N/A/V | E322N/L/T/S/K |
| 52 | Canary | — | C153G/R | R187L/N | R309P/N/A/V | — |
| 53 | Neopelma | — | C154G/R | R188L/N | R310P/N/A/V | E311N/L/T/S/K |
| 54 | Alligator | — | — | R188L/N | R310P/N/A/V | E313N/L/T/S/K |
| 56 | Xenopus | — | — | R188L/N | R307P/N/A/V | E310N/L/T/S/K |
| 59 | Brown Trout | — | — | R188L/N | — | E310N/L/T/S/K |
| 61 | Electric eel | — | — | R188L/N | — | — |
| 63 | Walking fish | — | — | R188L/N | R305P/N/A/V | E308N/L/T/S/K |
| 65 | Guppy | — | — | R188L/N | R305P/N/A/V | E308N/L/T/S/K |
| 67 | Rat | — | — | R188L/N | R306P/N/A/V | E309N/L/T/S/K |
| 69 | Piliocolobus | — | — | R188L/N | R306P/N/A/V | E309N/L/T/S/K |
| 70 | Pig | M44R/Q | C154G/R | R188L/N | R306P/N/A/V | E309N/L/T/S/K |
| 73 | Water buffalo | M44R/Q | C154G/R | R188L/N | R305P/N/A/V | E308N/L/T/S/K |
| 74 | Marmot | M44R/Q | C154G/R | R188L/N | R306P/N/A/V | E309N/L/T/S/K |

In some embodiments, further TdT variants for use with methods of the invention include one or more of the substitutions of methionine, cysteine, arginine (first position), arginine (second position) or glutamic acid, as shown in Table 2.

In some embodiments, a TdT variant comprising an amino acid sequence at least ninety percent identical to an amino acid sequence of SEQ ID NOs 55, 57, 58, 60, 62, 64, 66, 68, 71, 72, and 75 through 112, inclusive, may also be used with the present invention.

TdT, PAP and PUP variants for use with the invention each comprise an amino acid sequence having a percent sequence identity with a specified SEQ ID NO, subject to the presence of indicated substitutions. In some embodiments, the number and type of sequence differences between a variant of the invention described in this manner and the specified SEQ ID NO may be due to substitutions, deletion and/or insertions, and the amino acids substituted, deleted and/or inserted may comprise any amino acid. In some embodiments, such deletions, substitutions and/or insertions comprise only naturally occurring amino acids. In some embodiments, substitutions comprise only conservative, or synonymous, amino acid changes, as described in Grantham, Science, 185: 862-864 (1974). That is, a substitution of an amino acid can occur only among members of its set of synonymous amino acids. In some embodiments, sets of synonymous amino acids that may be employed are set forth in Table 3A.

TABLE 3A
Synonymous Sets of Amino Acids I

| Amino Acid | Synonymous Set |
|---|---|
| Ser | Ser, Thr, Gly, Asn |
| Arg | Arg, Gln, Lys, Glu, His |
| Leu | Ile, Phe, Tyr, Met, Val, Leu |
| Pro | Gly, Ala, Thr, Pro |
| Thr | Pro, Ser, Ala, Gly, His, Gln, Thr |
| Ala | Gly, Thr, Pro, Ala |
| Val | Met, Tyr, Phe, Ile, Leu, Val |
| Gly | Gly, Ala, Thr, Pro, Ser |
| Ile | Met, Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Cys, Ser, Thr |
| His | His, Glu, Lys, Gln, Thr, Arg |
| Gln | Gln, Glu, Lys, Asn, His, Thr, Arg |
| Asn | Asn, Gln, Asp, Ser |
| Lys | Lys, Glu, Gln, His, Arg |
| Asp | Asp, Glu, Asn |
| Glu | Glu, Asp, Lys, Asn, Gln, His, Arg |
| Met | Met, Phe, Ile, Val, Leu |
| Trp | Trp |

In some embodiments, sets of synonymous amino acids that may be employed are set forth in Table 3B.

TABLE 3B
Synonymous Sets of Amino Acids II

| Amino Acid | Synonymous Set |
|---|---|
| Ser | Ser |
| Arg | Arg, Lys, His |
| Leu | Ile, Phe, Met, Leu |
| Pro | Ala, Pro |
| Thr | Thr |
| Ala | Pro, Ala |
| Val | Met, Ile Val |
| Gly | Gly |
| Ile | Met, Phe, Val, Leu, Ile |
| Phe | Met, Tyr, Ile, Leu, Phe |
| Tyr | Trp, Met |
| Cys | Cys, Ser |
| His | His, Gln, Arg |
| Gln | Gln, Glu, His |
| Asn | Asn, Asp |
| Lys | Lys, Arg |
| Asp | Asp, Asn |
| Glu | Glu, Gln |
| Met | Met, Phe, Ile, Val, Leu |
| Trp | Trp |

TdT, PAP and PUP variants for use with the invention are produced by conventional biotechnology techniques and may include an affinity tag for purification, which may be attached to the N-terminus. C-terminus or at an interior position of the template-free polymerase. In some embodiments, affinity tags are cleaved before the template-free polymerase is used. In other embodiments, affinity tags are not cleaved before use. In some embodiments, a peptide affinity tag is inserted into a loop 2 region of a TdT variant. An exemplary N-terminal His-tag for use with TdT variants of the invention is MASSHHHHHHSSGSENLYFQTGSSG-(SEQ ID NO: 6)). Guidance for selecting a peptide affinity tag is described in the following references: Terpe. Appl. Microbiol. Biotechnol., 60: 523-533 (2003); Arnau et al. Protein Expression and Purification, 48: 1-13 (2006); Kimple et al. Curr. Protoc. Protein Sci., 73: Unit-9.9 (2015); Kimple et al, U.S. Pat. No. 7,309,575; Lichty et al, Protein Expression and Purification, 41: 98-105 (2005); and the like. Guidance for selecting a peptide affinity tag is described in the following references: Terpe. Appl. Microbiol. Biotechnol., 60: 523-533 (2003); Arnau et al, Protein Expression and Purification, 48: 1-13 (2006); Kimple et al. Curr. Protoc. Protein Sci., 73: Unit-9.9 (2015); Kimple et al. U.S. Pat. No. 7,309,575; Lichty et al, Protein Expression and Purification, 41: 98-105 (2005); and the like.

### Measurement of Nucleotide Incorporation Activity

The efficiency of nucleotide incorporation by variants used with the invention may be measured by an extension, or elongation, assay, e.g. as described in Boule et al (cited below); Bentolila et al (cited below); and Hiatt ct al, U.S. Pat. No. 5,808,045, the latter of which is incorporated herein by reference. Briefly, in one form of such an assay, a fluorescently labeled oligonucleotide having a free 3-hydroxyl is reacted with a template-free polymerase, such as a TdT, under extension conditions for a predetermined duration in the presence of a reversibly blocked nucleoside triphosphate, after which the extension reaction is stopped and the amounts of extension products and unextended oligonucleotide are quantified after separation by gel electrophoresis. By such assays, the incorporation efficiency of a variant template-free polymerase may be readily compared to the efficiencies of other variants or to that of wild type or reference polymerases. In some embodiments, a measure of template-free polymerase efficiency may be a ratio (given as a percentage) of amount of extended product using the variant template-free polymerase over the amount of extended product using wild type template-free polymerase, or reference polymerase, in an equivalent assay.

In some embodiments, the following particular extension assay may be used to measure incorporation efficiencies of TdTs: The primer used is the following:
5′-AAAAAAAAAAAAAAGGGG-3′ (SEQ ID NO: 5)
   The primer has also an ATTO fluorescent dye on the 5′ extremity. Representative modified nucleotides used (noted as dNTP in Table 6) include 3′-O-amino-2′,3′-dideoxynucleotides-5′-triphosphates (—ONH₂, Firebird Biosciences), such as 3′-O-amino-2′,3′-dideoxyadenosine-5′-triphosphate. For each different variant tested, one tube is used for the reaction. The reagents are added to the tube, starting from water, and then in the order of Table 4. After 30 min at 37° C. the reaction is stopped by addition of formamide (Sigma).

TABLE 4
Extension Activity Assay Reagents

| | | |
|---|---|---|
| Reagent | Concentration | Volume |

| | | |
|---|---|---|
| H₂O | — | 12 μL |
| Activity buffer | 10x | 2 μL |

| | | | |
|---|---|---|---|
| dNTP | 250 | μM | 2 μL |
| Purified enzyme | 20 | μM | 2 μL |
| Fluorescent primer | 500 | nM | 2 μL |

The Activity buffer comprises, for example, TdT reaction buffer (available from New England Biolabs) supplemented with CoCl₂.

The product of the assay is analyzed by conventional polyacrylamide gel electrophoresis. For example, products of the above assay may be analyzed in a 16 percent polyacrylamide denaturing gel (Bio-Rad). Gels are made just before the analysis by pouring polyacrylamide inside glass plates and let it polymerize. The gel inside the glass plates is mounted on an adapted tank filed with TBE buffer (Sigma) for the electrophoresis step. The samples to be analyzed are loaded on the top of the gel. A voltage of 500 to 2,000V is applied between the top and bottom of the gel for 3 to 6 h at room temperature. After separation, gel fluorescence is scanned using, for example, a Typhoon scanner (GE Life Sciences). The gel image is analyzed using ImageJ software (imagej.nih.gov/ij/), or its equivalent, to calculate the percentage of incorporation of the modified nucleotides.

The elongation efficiency of a template-free polymerase may also be measured in the following hairpin completion assay. In such assay, a test polynucleotide is provided with a free 3′ hydroxyl such that under reaction conditions it is substantially only single stranded, but that upon extension with a polymerase, such as a TdT variant, it forms a stable hairpin structure comprising a single stranded loop and a double stranded stem. This allows the detection of an extension of the 3′ end by the presence of the double stranded polynucleotide. The double stranded structure may be detected in a variety of ways including, but not limited to, (i) fluorescent dyes that preferentially fluoresce upon intercalation into the double stranded structure, (ii) fluorescent resonance energy transfer (FRET) between an acceptor (or donor) on the extended polynucleotide and a donor (or acceptor) on an oligonucleotide that forms a triplex with the newly formed hairpin stem, (iii) FRET acceptors and donors that are both attached to the test polynucleotide and that are brought into FRET proximity upon formation of a hairpin, or the like. In some embodiments, a stem portion of a test polynucleotide after extension by a single nucleotide is in the range of 4 to 6 basepairs in length; in other embodiments, such stem portion is 4 to 5 basepairs in length; and in still other embodiments, such stem portion is 4 basepairs in length. In some embodiments, a test polynucleotide has a length in the range of from 10 to 20 nucleotides; in other embodiments, a test polynucleotide has a length in the range of from 12 to 15 nucleotides. In some embodiments, it is advantageous or convenient to extend the test polynucleotide with a nucleotide that maximizes the difference between the melting temperatures of the stem without extension and the stem with extension; thus, in some embodiments, a test polynucleotide is extended with a dC or dG (and accordingly the test polynucleotide is selected to have an appropriate complementary nucleotide for stem formation).

Exemplary test polynucleotides for hairpin completion assays include p875 (5′-CAGTTAAAAACT) (SEQ ID NO: 2) which is completed by extending with a dGTP; p876 (5′-GAGTTAAAACT) (SEQ ID NO: 3) which is completed by extending with a dCTP; and p877 (5′-CAGCAAGGCT) (SEQ ID NO: 4) which is completed by extending with a dGTP. Exemplary reaction conditions for such test polynucleotides may comprise: 2.5-5 μM of test polynucleotide, 1:4000 dilution of GelRed* (intercalating dye from Biotium, Inc., Fremont. CA), 200 mM Cacodylate KOH pH 6.8, 1 mM CoCl2, 0-20% of DMSO and 3′-ONH₂ dGTP and TdT at desired concentrations. Completion of the hairpin may be monitored by an increase in fluorescence of GelRed® dye using a conventional fluorimeter, such as a TECAN reader at a reaction temperature of 28-38° C. using an excitation filter set to 360 nm and an emission filter set to 635 nm.

### Template-Free Polymerase Inks

As mentioned above, synthesis reagents delivered by inkjet pumps must be formulated so as to preserve activity of synthesis reagents and to meet the rheological requirements for droplet formation. Such formulations are referred to herein as “inks.” For example, satisfying the first constraint (activity) may require that a template-free polymerase be present in a reaction mixture at a certain minimal concentration. However, because of high protein viscosity, the concentration for the desired activity may prevent satisfaction of the second constraint, i.e. capability of droplet formation. In such cases, embodiments of the invention may call for delivery of a plurality of droplets each with lower concentrations of polymerase, which coupled with evaporation permit the build-up of polymerase concentration for a desired level of activity. Such an embodiment is illustrated in FIG. 2D.

As also mentioned above, the key solution parameters affecting droplet formation by inkjets are viscosity, surface tension, density and diameter of the inkjet nozzle, which are related through the formula: Z=[(ργa)\hat{ }(0.5)]/η, where ρ is the density of the fluid, γ is surface tension, η is viscosity, a is the radius of the inkjet pump nozzle and Z is in the range of from 1 to 10 for reliable droplet formation. e.g. Derby, J. Mater. Chem., 18: 5717-5721 (2008). This relationship applies to any of the synthesis reagents delivered by inkjet-generated droplets, including (i) template-free polymerase in its coupling buffer, (ii) a mixture of template-free polymerase in its coupling buffer and a 3′-O-protected-dNTP, (iii) a 3′-O-protected-dNTP in a buffer, (iv) a deprotection buffer, and (v) a buffer containing a 5′-linker-derivatized initiator. Applying this relationship to determine ink compositions that are capable of forming desired droplets for particular embodiments may be carried out by one of ordinary skill in the art by adjusting densities of reactants, viscosity modifiers, surface tension modifiers, and the like.

In some embodiments, an elongation reaction buffer is an acetate buffer, for example, 0.1 M acetate, 0.5 M NaCl, pH 4.5.

Thus, for particular embodiments of the invention, synthesis reagents prepared for non-droplet delivery to a reaction mixture may be reformulated by adding viscosity modifiers, surface tension modifiers and density modifiers, and the like, in order to form “printable inks” that may be delivered in droplets generated by inkjet pumps. “Printable” in reference to a reagent ink means repeatable droplets are able to be ejected from the nozzle, with uniform velocities and volumes and no satellite droplets.

In some embodiments, if the specific activity of a template-free polymerase is relatively low, so that a relatively large amount of protein must be delivered to reaction sites to complete a coupling step, then the delivery of the polymerase may be carried out by dispensing a plurality of droplets in each coupling cycle together with allowing a controlled amount of evaporation to maintain a reaction volume within a prescribed range. e.g. 10-100 μL. In some embodiments, the plurality of droplets delivered is in the range of from 2 to 10, or in the range of from 2 to 5, or in the range of from 2 to 3. In other embodiments, the plurality of droplets may be in the range of from 2 to 150, or in the range of from 10 to 120. In some embodiments, whenever the template-free polymerase is a TdT, the plurality of droplets is the number required to bring the concentration of TdT in the reaction mixture at a reaction site to a value in the range of from 1 μM to 30 μM, or in the range of from 2 μM to 20 μM. In some embodiments, a concentration of TdT in an ink is the concentration that produces an approximate 1:1 stoichiometry between TdT molecules and polynucleotides at a reaction site. In other embodiments, a concentration of TdT in an ink is a concentration that produces a stoichiometry between TdT molecules and polynucleotides at a reaction site that is 1:1 or greater.

In some embodiments, the invention includes a printable ink comprising a TdT variant and a viscosity modifier. In some embodiments, such TdT in a concentration in the range of from 1 μM to 20 μM/mg in a buffer suitable for coupling activity. In some embodiments, such buffer comprises about 10 to about 500 mM potassium cacodylate buffer (pH between 6.5 and 7.5) and from about 0.01 to about 10 mM of a divalent cation (e.g. CoCl₂ or MnCl₂). In some embodiments, TdT inks are characterized by a viscosity of about 1 centipoise (0.001 Pa-sec) to about 20 centipoise (0.02 Pa-sec) when viscosity is measured at room temperature; and a surface tension of about 15 dynes/cm and about 50 dynes/cm when measured at room temperature. In some embodiments, the viscosity modifier is selected from the group consisting of glycerol, ethylene glycol, polyethylene glycol of different molecular weights, glycerol, poly(vinyl alcohol), carboxymethyl cellulose and hydroxyethyl cellulose. In some embodiments, a template-free polymerase ink, such as a TdT ink, comprises glycerol at a concentration in the range of from 5 percent (w/w) to 55 percent (w/w); in other embodiments, a template-free polymerase ink, such as a TdT ink, comprises glycerol at a concentration in the range of from 5 percent (w/w) to 40 percent (w/w); in other embodiments, a template-free polymerase ink, such as a TdT ink, comprises glycerol at a concentration in the range of from 5 percent (w/w) to 25 percent (w/w); in other embodiments, a TdT ink comprises glycerol at a concentration in the range of from 8 percent (w/w) to 40 percent (w/w); in other embodiments, a TdT ink comprises glycerol at a concentration in the range of from 8 percent (w/w) to 25 percent (w/w); in other embodiments, a TdT ink comprises glycerol at a concentration in the range of from 10 percent (w/w) to 40 percent (w/w). It is understood by those of ordinary skill that the viscosity ranges achieved by the above glycerol concentration ranges also may be achieved by equivalent concentration ranges of other viscosity modifiers. Thus, in some embodiments, a TdT ink comprises a concentration of a viscosity modifier that produces an equivalent viscosity as glycerol at a concentration in the range of from 10 percent (W/W) to 40 percent (w/w). In addition to glycerol, of particular interest are the viscosity modifiers carboxymethyl cellulose and hydroxyethyl cellulose which have minimal effect on template-free polymerase coupling activity, such as TdT coupling activity. In some embodiments, besides a viscosity modifier, the printable template-free polymerase ink, such as a TdT ink, comprises a surface tension modifier. Such surface tension modifier may be a detergent. Such detergent may be selected from Tween 20, Triton X-100, CHAPS, NP-40, octyl thioglycoside, octyl glucoside or dodecyl maltoside. Of particular interest is Triton X-100. Also of particular interest is Tween 20. Additional surface tension modifiers (i.e. surfactants) are disclosed in Buret, LabChip, 12: 422-433 (2012).

In some embodiments, the invention includes a printable ink comprising a TdT variant, a 3′-O-protected-dNTP and a viscosity modifier. In some embodiments, such TdT is in a concentration in the range of from 1 μM to 50 μM/mg, or 1 μM to 20 μM/mg, in a buffer suitable for coupling activity. In some embodiments, such buffer comprises about 10 to about 500 mM potassium cacodylate buffer (pH between 6.5 and 7.5) and from about 0.01 to about 10 mM of a divalent cation (e.g. COCl₂ or MnCl₂); such 3′-O-protected-dNTP is in a concentration in the range of 125-600 μM. In some embodiments, the viscosity modifier is selected from the group consisting of glycerol, ethylene glycol, polyethylene glycol of different molecular weights, glycerol, poly(vinyl alcohol), carboxymethyl cellulose and hydroxyethyl cellulose. In some embodiments, besides a viscosity modifier, the printable TdT ink comprises a surface tension modifier. Such surface tension modifier may be a detergent. Such detergent may be selected from Triton X-100, CHAPS, NP-40, octyl thioglycoside, octyl glucoside or dodecyl maltoside. Of particular interest are Tween 20 and Triton X-100.

In each of the above embodiments of TdT inks, a TdT has an amino acid sequence that is at least 80 percent identical to a TdT variant selected disclosed herein. In each of the above embodiments of TdT inks, a TdT has an amino acid sequence that is at least 80 percent identical to a TdT variant selected from Table 1 or Table 2. In some embodiments, a TdT ink of the invention comprises a TdT with an amino acid sequence that is at least 90 percent identical to a TdT variant disclosed herein. In some embodiments, a TdT ink of the invention comprises a TdT with an amino acid sequence that is at least 90 percent identical to a TdT variant selected from Table 1 or Table 2.

In some embodiments, inks of synthesis reagents may be formulated as emulsions. In particular. TdT inks of the invention may be emulsions.

In some embodiments, a printable ink comprising a template-free polymerase, such as a TdT variant, includes a humectant for reducing droplet evaporation. Suitable humectants include, but are not limited to, glycerol, alcohol sugars, ethylhexylglycerin, panthenol, sorbitol, xylitol, maltitol, propylene glycol, hexylene glycol, butylene glycol, sodium lactate, hyaluronic acid, and polydextrose,

In some embodiments, a TdT ink of the invention is delivered in a droplet in range of from 1 μL to 200 μL, or from 1 μL to 100 μL, or from 1 μL-50 μL.

In some embodiments, the invention is directed to a terminal deoxynucleotidyl transferase (TdT) composition comprising a droplet of an aqueous solution having a volume in the range of from 2 μL to 5 nL and comprising (i) a TdT or variant thereof in a concentration in the range of from 1.0 μM to 30 μM, or in the range of from 2.0 μM to 20 μM, a divalent cation in a concentration in the range of from 0.01 to 10 mM, and a viscosity modifier. In some embodiments, the divalent cation is cobalt or manganese and such composition further comprises a surface tension modifier. In some embodiments, such viscosity modifier is selected from the group consisting of glycerol, ethylene glycol, polyethylene glycol, poly(vinyl alcohol), carboxymethyl cellulose and hydroxyethyl cellulose. In some embodiments, any of the above compositions may further comprise an aldehyde scavenger (described more fully below) whenever the printable template-free polymerase ink, such as a printable TdT ink, comprises a 3′O-amino-nucleotide. In some embodiments, any of the above compositions may further comprise a 3′-O-protected-2′-deoxynucleoside triphosphate monomer in a concentration in the range of from 100-1000 μM, or from 125-600 μM.

In some embodiments, the invention is directed to a 3′-O-protected-2′-deoxynucleoside triphosphate composition comprising a droplet of an aqueous solution having a volume in the range of from 2 μL to 5 nL and comprising (i) a 3′-O-protected-2′-deoxynucleoside triphosphate in a concentration in the range of from 125-600 μM. and a viscosity modifier. In some embodiments, the foregoing 3′-O-protected-2′-deoxynucleoside triphosphate composition further comprising a surface tension modifier. In some embodiments of the foregoing compositions, the viscosity modifier is selected from the group consisting of glycerol, ethylene glycol, polyethylene glycol, poly(vinyl alcohol), carboxymethyl cellulose and hydroxyethyl cellulose. In some embodiments, the 3′-O-protected-2′-deoxynucleoside triphosphate of the foregoing composition is a 3′-O-protected-2′-deoxyadenosine triphosphate, a 3′-O-protected-2′-deoxyguanosine triphosphate, a 3′-O-protected-2′-deoxycytidine triphosphate, or a 3′-O-protected-2′-deoxythymidine triphosphate. In some embodiments of the foregoing composition, the 3′-O-protection group is selected from the group consisting of 3′-O-methyl, 3′-O-(2-nitrobenzyl), 3′-O-allyl, 3′-O-amine, 3′-O-azidomethyl, 3′-O-tert-butoxy ethoxy, 3′-O-(2-cyanoethyl), and 3′-O-propargyl.

In some embodiments, the invention is directed to a printable 3′-O-protected-2′-deoxynucleoside triphosphate ink, comprising: an aqueous solution of a 3′-O-protected-2′-deoxynucleoside triphosphate having a concentration in a range of from 125 μM to 600 μM; wherein whenever the ink is printed to a substrate, printed droplets each have a volume in the range of 0.1 μL to 5 nL of the aqueous 3′-O-protected-2′-deoxynucleoside triphosphate solution and is characterized by a viscosity of about 1 centipoise to about 20 centipoise when viscosity is measured at room temperature; and a surface tension of about 15 dynes/cm and about 50 dynes/cm when measured at room temperature.

As mentioned above, in some embodiments of the invention where 3′-O-amino-dNTP monomers are employed, the presence of an aldehyde scavenger in the template-free polymerase inks reduces spurious capping of the 3′-amines by reaction with adventitious aldehydes or ketones, such as formaldehyde, which are pervasive in the environment. This is a special problem with inkjet synthesis because droplets of ink have very high surface-to-volume ratios that enhances absorption of environmental aldehydes. Thus, in embodiments of the invention employing 3′-O-amino-dNTP monomers, template-free polymerase inks as described above further include an effective amount of at least one aldehyde scavenger. As used herein, “effective amount” in reference to an aldehyde scavenger means an amount (or concentration) sufficient to produce a measurable decrease in spuriously capped polynucleotides in a product. Such measurements may be made readily using conventional techniques. e.g. DNA sequence analysis of a sample of a product, gel electrophoresis, or the like. As used herein, the term “aldehyde scavenger” includes ketone scavengers. In some embodiments, aldehyde scavengers are agents that react with compounds having chemical groups of the formula R—C(═O)H or R¹—C(═O)—R², where R, R¹ and R² are typically alkyl or aryl. More particularly, in some embodiments, aldehyde scavengers are agents that react with R—C(═O)H or R¹—C(═O)—R² groups on compounds at a sufficiently high rate that such compounds do not react with (or react only negligibly with) the 3′-amine group of 3′-O-amino-nucleotides. As used herein, the term “scavenger” means a chemical substance added to a mixture in order to remove or de-activate impurities or compounds that lead unwanted reaction products. In various embodiments, aldehyde scavengers may be in solution, immobilized on the materials used for storage or synthesis or coupled to reagents employed in method of the invention, for example, template-free polymerases, such as TdTs.

As noted above, enzymatic synthesis may be carried out using a variety of reagents (referred to herein as “synthesis reagents”) that may contain or consist of reactants, wash solutions, deprotection buffers, enzymes, and the like. (The term “synthesis reagent” means any reagent used in a synthesis cycle to couple a monomer, particularly a 3′-O-amino-nucleoside triphosphate, to an initiator or elongated fragment, such as, buffers comprising a template-free polymerase, buffers comprising 3′-O-protected-nucleotide monomers, deprotection (or deblocking) buffers, and the like.) In various embodiments, an aldehyde scavenger may be a component of one or more of the synthesis reagents. In some embodiments, an aldehyde scavenger may be added to a reaction mixture as a separate synthesis reagent (without other reactants, wash buffers or enzymes). In some embodiments, an aldehyde scavenger is added to a reaction mixture as a component of a synthesis reagent comprising a template-free polymerase.

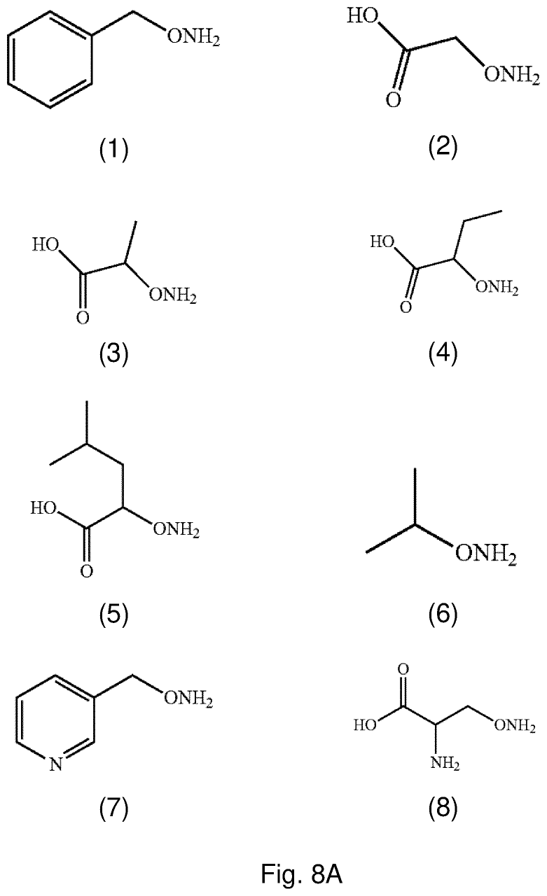

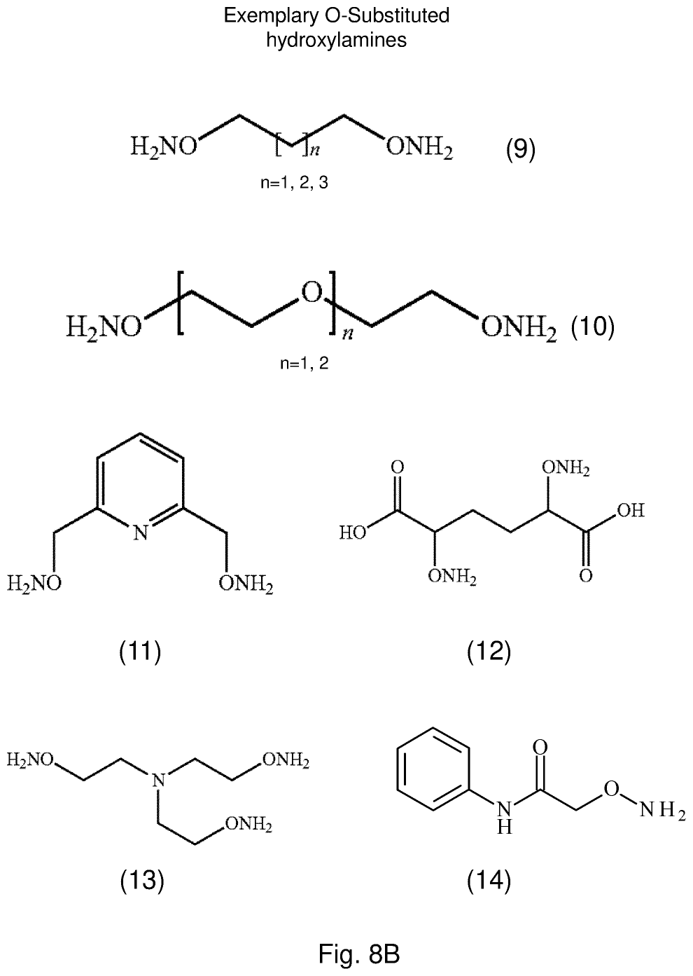

In some embodiments. e.g. employing aldehyde scavengers disclosed by Sudo et al. U.S. patent publication US2020/0061225 or listed in FIGS. 8A-8B, an effective amount is provided by a concentration in the range of from 1 to 500 mM, or in other embodiments in the range of from 1 to 200 mM, or in other embodiments in the range of from 1 to 100 mM.

In some embodiments, aldehyde scavengers employed in the invention comprise O-substituted hydroxylamines or polyhydroxylamines. In some embodiments. O-substituted hydroxylamines used in the invention are defined by the formula:

R¹—ONH₂

such as disclosed by Sudo et al, U.S. patent publication US2020/0061225, or Kitasaka et al. U.S. Pat. No. 7,241,625, which are incorporated herein by reference. In some embodiments, R¹ is a C₁₋₁₈ linear, branched or cyclic alkyl group which may be substituted by at least one substituent selected from the group consisting of a halogen atom: a C₁₋₆ alkyloxy group; a C₁₋₆ haloalkyl group; a C₁₋₆ haloalkyloxy group; a carboxy group; a hydroxy group; a mercapto group; a cyano group: a nitro group: a C₆₋₁₄ aryl group which may be substituted by a halogen atom, a C₁₋₆ alkyl group, a C₁₋₆ alkyloxy group, a C₁₋₆ haloalkyl group, a C₁₋₆ haloalkyloxy group, a carboxy group, a hydroxy group, a mercapto group, a cyano group or a nitro group; a C₄₋₁₄ heteroaryl group which may be substituted by a halogen atom, a C₁₋₆ alkyl group, a C₁₋₆alkyloxy group, a C₁₋₆ haloalkyl group, a C₁₋₆ haloalkyloxy group, a carboxy group, a hydroxy group, a mercapto group, a cyano group or a nitro group; an alkoxycarbonyl group represented by the following formula:

—(C═O)—O—R²

and a carbamoyl group represented by the following formula:

—(C═O)—NR³(R³)

wherein R² is a C₁₋₁₈ linear, branched or cyclic alkyl group which may be substituted, at a chemically acceptable optional position, by at least one substituent selected from the group consisting of a carboxy group; a hydroxy group; a mercapto group; a halogen atom; a C₁₋₆ alkyloxy group; a C₁₋₆ haloalkyloxy group; a C₆₋₁₄ aryl group; and a C₄₋₁₄ heteroaryl group; and wherein each R³ may be the same or different and each independently a C₁₋₁₈ linear, branched or cyclic alkyl group which may be substituted by at least one substituent selected from the group consisting of a carboxy group; a hydroxy group; a mercapto group; a halogen atom; a C₁₋₆ alkyloxy group; a C₁₋₆ haloalkyloxy group; a C₆₋₁₄ aryl group; and a C₄₋₁₄ heteroaryl group; a C₆₋₁₄ aryl group, a C₄₋₁₄ heteroaryl group, or a hydrogen atom.

In particular, exemplary O-substituted hydroxylamines or polyhydroxylamines which may be used in the invention are shown as compounds (1)-(14) in FIGS. 3A and 3B, wherein compound (1) is also referred to herein as the “BOX” reagent.

In some embodiments aldehyde scavengers comprise carbonyl compounds disclosed by Pacifici, U.S. Pat. No. 5,446,195 or Burdeniuc et al. U.S. patent publication, US20160369035; which are incorporated herein by reference, and are defined by the formula:

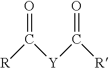

wherein R and R′ are CH3 or H[O(CH2)m]nO- and wherein m and n are selected from the group of combinations of m and n consisting of: m=1 and n=1, 3-19; m=2 and n=2-19; or m=3 and n=1-19, Y is —CH2— or —CH2—CO—CH2—.

In some embodiments of the invention, template-free polymerase inks as described above further include a dye to permit monitoring of the location, size, shape and possible overlap of reaction sites, either at an initial dispensing of reagents to define the reaction sites or at subsequent droplet dispensations during synthesis, particularly to monitor possible coalescence of reaction mixtures at adjacent sites. A large selection of fluorescent and non-fluorescent dyes are available for this purpose. The main criteria for use is that the dye (i) not adversely affect the performance of any reaction component, (ii) be bright or concentrated enough to make droplets or reaction sites readily detectable, (iii) be spectrally distinct if more than one is used, and (iv) not affect the rheological properties of the ink. In some embodiments, food dyes are used in inks of the invention. In other embodiments, pH indicator dyes are used in inks of the invention. In other embodiments, fluorescent dyes are used in inks of the invention. Exemplary dyes for use with template-free polymerase inks include Brilliant Blue FCF. Fast Green FCF, Ponceau 4R and Sunset Yellow FCF. In some embodiments, food dyes are used at a concentration in the range of from 1 to 20 mM, or at a concentration in the range of from 1 to 10 mM.

### Example 1

### Methods for Evaluating Reaction Conditions

### In Enzymatic Inkjet Synthesis

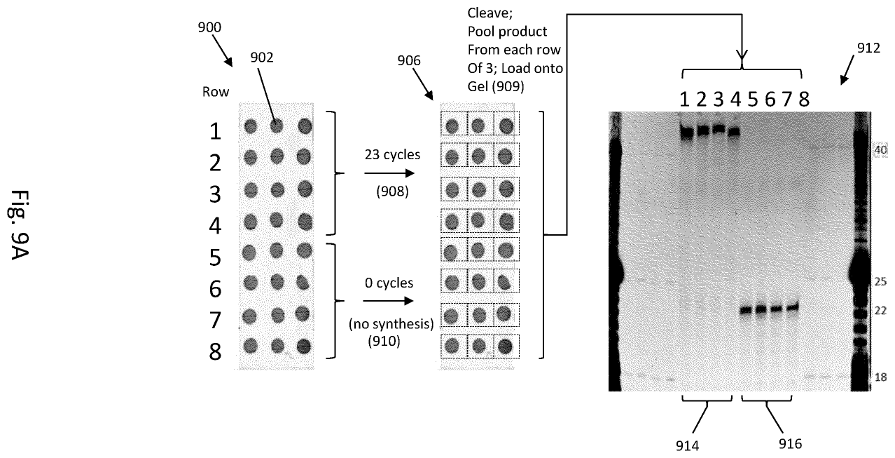

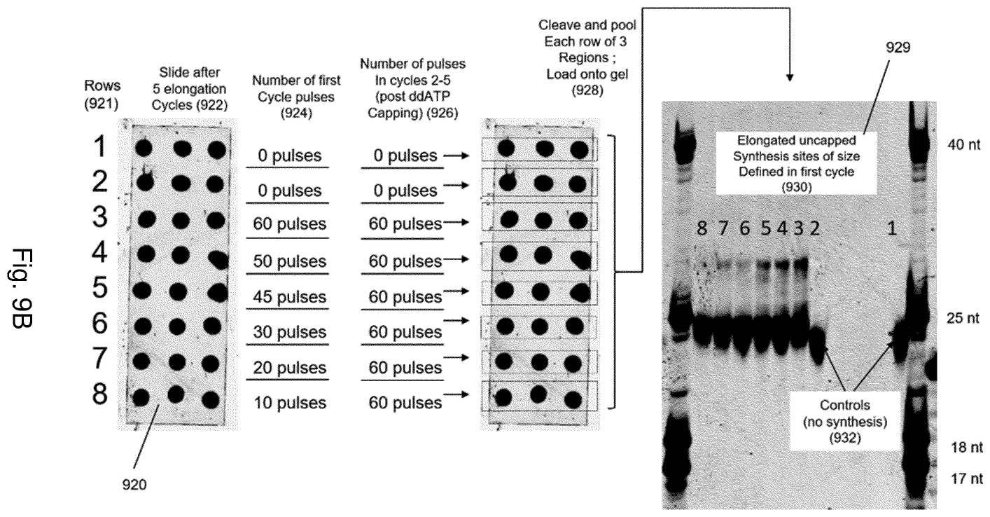

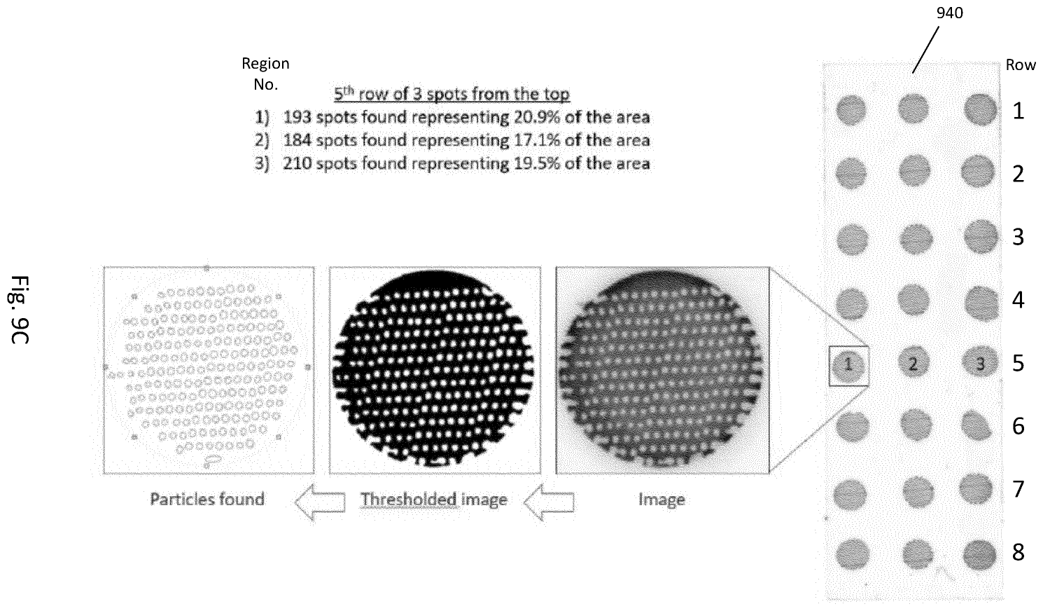

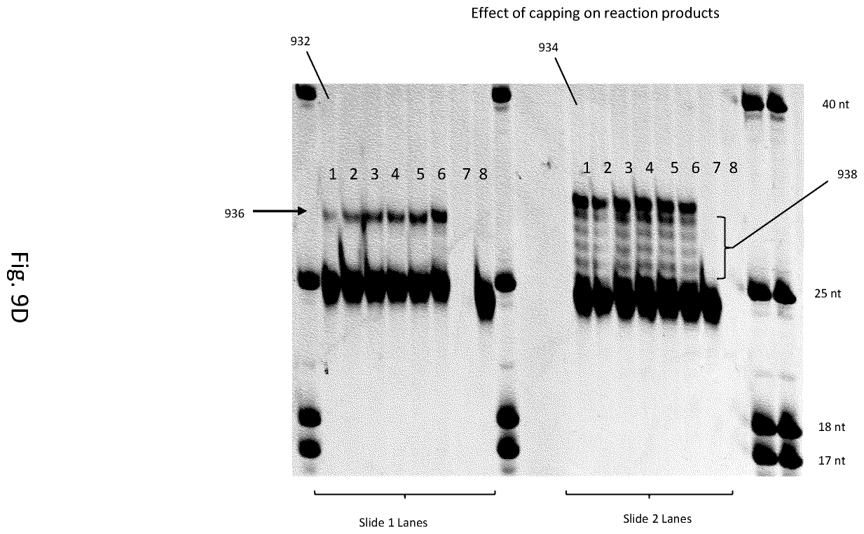

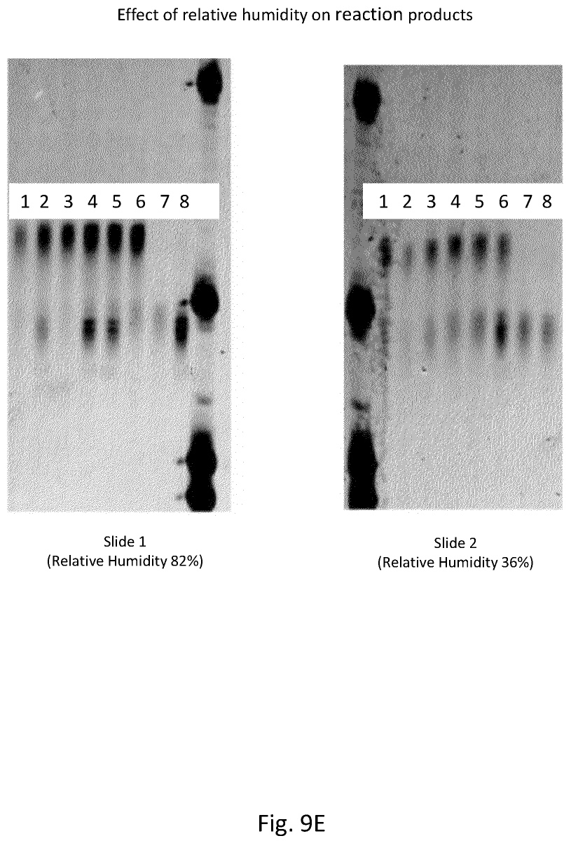

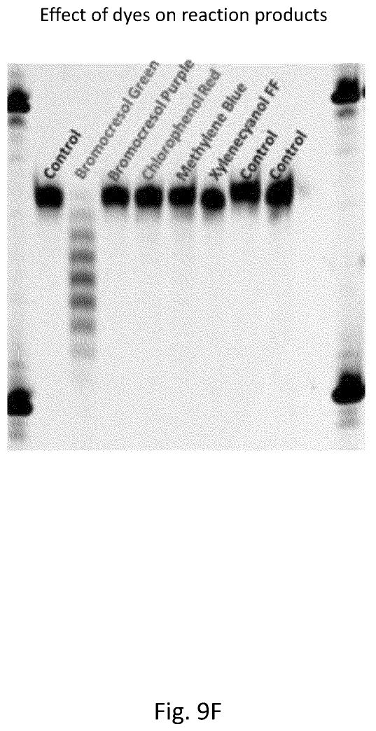

In this example, a test bed was created for evaluating different inkjet reaction conditions. Because the very small amounts of materials at individual reaction sites are difficult to analyze, slides were prepared that allowed regions containing large numbers of reaction sites (e.g. 10 or more) to be processed together and pooled for analysis by gel electrophoresis. The regions were created by depositing equal volumes of alkali- or photo-cleavable initiators in each of the regions. An exemplary slide (900) (which may be a PolyAn 3D-epoxy coated glass slide) is shown in FIG. 9A. It contains 24 circular regions (902) each of which contains approximately the same number of reaction sites (not shown). Regions (902) were created on slide (900) by hand spotting 20 μM 5′-amino-derivatized photo-cleavable initiators to the 24 locations (following the slide manufacturer's recommended protocol). Briefly, after incubation overnight at 70% relative humidity, the slide is then heated to 80° C. for 5 min, washed for 1 h in 1M ethanolamine pH 8, 30 min in SSC 4×, 30 min in SSC 2×0.1% SDS, 30 min in SSC 0.2×, 30 min in MQ. Exemplary initiators may have the following sequence: 5′-amino-C12-10T(PC)4T(FAM-T)18T-3′, where C12 is a 12-carbon alkyl linker, T is thymidine, “PC” is a photo-cleavable linker (e.g. Horgan, WO2021/048142) and “FAM-T” is a fluorescein-labeled thymidine. After an experiment is carried out, sequences are photo-cleaved from specific regions by immersing the region(s) in PBS (e.g. 40 μL/region) and illuminating the region(s) with 365 nm light (e.g. Analytik Jena, 95-0252-02. UVLMS-38, 8W-intensity a 3″ is 1500 μW/cm² for 365 nm), after which the pooled sequences are loaded on a gel as diagrammed in FIG. 9A. In the example of FIG. 9A, regions are grouped into eight groups of 3 regions each. In groups 1-4 23 cycles of elongation reactions are carried out, after which the resulting product in each group of three are photo-cleaved, pooled and loaded (909) on their respective lanes 1-4 (914). In groups 5-8 no synthesis reactions are carried out. The non-elongated initiators are processed exactly the same as groups 1-4, but are loaded into lanes 5-8 (916). The gel provides a convenient and sensitive measure of the effects of changes in various reaction parameters, including but not limited to, reactant concentrations, the presence, absence and concentration of rheological ingredients (such as, surfactants, viscosity modifiers, and so on), different template-free polymerases, secondary structure modifiers, and the like.

### Example 2

### Five Cycles of Enzymatic Synthesis Using

### Move-Stop Reagent Deposition

In this Example, a Fujifilm Dimatix Samba recirculating silicon MEMS print head was used to deliver in “move-stop” or “jetting” mode reagent droplets in 5 cycles of enzymatic synthesis. (Capping and deblocking steps were performed manually). The recirculating feature was employed to address problems due to ink drying on nozzles. The “move-stop” feature was employed to improve deposition accuracy which is especially important in the synthesis of high density arrays of polynucleotides. In the “move-stop” or “jetting” mode, the print head stops above each target reaction site (i.e. a reaction site programmed to receive a synthesis reagent), deposits a predetermined number of droplets (sometimes referred to herein as “pulses”) at the reaction site, then moves to the next target reaction site programmed to receive a deposition. Using this mode reaction site size (e.g. diameter) and the amount of synthesis reagent delivered can be determined by programming individual nozzles to deliver a predetermined number of droplets or pulses. Droplet sizes are predetermined and in the case of the Samba print head, droplet volumes are each about 2.4 μL. Different amounts of enzyme buffer (22% w/w glycerol, 20 mM BOX aldehyde scavenger, 20 μM TdT (M57), 500 μM dTTP-3′ONH₂) were deposited onto a PolyAn 3D epoxy slide with 24 (8 rows of 3 spots) FAM-labelled photocleavable DNA spots (as described in Example 1) approximately 3 mm in diameter using the jetting mode of the Dimatix Samba print head, 1024 of the 2048 nozzles of the Samba print head were used; in synthesis cycle 1: 10-60 pulses of reagent were delivered depending on reaction site location; in synthesis cycles 2-5 60 pulses were delivered at each reaction site; the print head was operated in move-stop (i.e. jetting) mode with 5 kHz firing, tickle pulse non-printing waveform; 268, 38 and 355 inlet, return and meniscus pressure, respectively.

After printing enzyme buffer, the slide was removed from the print stage and incubated 10 minutes in a humidity chamber, 5 μL capping solution (500 μM ddATP, 10 μM TdT (M57 (SEQ ID NO: 102) in TdT elongation buffer) (a 0.09% Tween 20 in MQ-water solution was used as a diluent)) was then pipetted onto the 6 rows of DNA spots (i.e. rows 3 through 8 of FIG. 9B) that had been printed on and left to incubate 10 minutes. The slide was then placed into a 50 mL centrifuge tube containing deprotection buffer (1.3 M NaNO₂, 0.7 M NaNO2, pH 5.2) for 3 minutes with gentle agitation, then rinsed with MQ-water under a tap and incubated 1 minute in 50 mL centrifuge tube of MQ-water, then rinsed again under a MQ-water tap and dried with Argon. The slide was then placed back onto the stage, its position verified using the fiducial mark and camera and the next cycle performed. Instead of using different numbers of pulses in different positions in cycles 2 to 5, 60 pulses were used for each of the 6 rows. After synthesis, DNA spots were cleaved 15 min in PBS (10 uL per 3 mm DNA spot) under UV illumination at 365 nm, spots of the same row were then pooled and 10 μL bromophenol blue stain added before gel electrophoresis. The results are shown in FIG. 9B, where slide (920) (prepared essentially as described in Example 1) is shown with eight rows (921) of three regions each after synthesis was completed (922). The size of reaction sites within each region were determined by the number of pulses delivered in the first synthesis cycle (924), after which reagent was delivered to each reaction site by 60 pulses (926). After the five cycles were completed, reaction sites of each row of three regions was cleaved, pooled and loaded (928) onto gel (929), with the rows of slide (920) corresponding to the lanes indicated on gel (929). The amount of product indicated in lanes 8 to 3 is monotonically related to the number of pulses used to define the size of the reaction sites.

### Example 3

### Density of Reaction Sites in Enzymatic Synthesis

### Using Move-Stop Reagent Deposition

In this example, synthesis (5 cycles) was carried out as described above by move-stop-jetting onto a glass slide with 24 (8 rows×3 columns) large (ca. 4 mm) FAM-labelled, photocleavable DNA regions. Synthesis and cleaving were done as described above. The only thing different was the reaction site (i.e. “spot”) density within the 3-region rows, 60 pulses in cycle 1 resulted in enough elongated material to be detectable on a gel, move-stop-jetting tests to increase the density of spots were done with 60 pulses. Instead of moving the substrate 8 times vertically under the print head and each time jetting, in this example, the substrate was moved 15 times i.e. in 5 mm increments. The slide was then shifted laterally 232 μm and the move-stop-jetting process repeated. This was repeated a further time to increase the reaction site density from 8880 to 26640 reaction sites/slide. FIG. 9C shows the slide (940) subjected to a negative end-labelling test performed with the synthesis ink. (That is, fluorescent labelling of unprinted areas was carried out with a fluorescent ddATP capping solution as per the capping protocol). No satellite spots (due to spurious droplet delivery) were observed. Using ImageJ image processing software, the size and number of reaction sites were determined in an automated manner (FIG. 9C) for the regions 1), 2) and 3) of row 5. The combined area of the unlabeled (jetted) spots also may be determined and compared with the area of the larger regions they are part of. The ratio obtained (about 19%) was then compared with the size of each band (of full length sequences) in the electropherogram (not shown) of product from a synthesis on a different slide carried out with an identical procedure (the ratio being about 25%). (Each lane is the sum of approximately 590 individual synthesis reactions (reaction site size about 100 μm, <4 fmol of DNA/reaction site). This confirms that 5-cycle synthesis can be performed on an array of reaction sites having a density of 26640 reaction sites in an area of a standard microscope slide.

The effect of TdT ink viscosity and volume of ink delivered to reaction sites was examined using the above experimental set-up. The volume of ink delivered to each reaction site determines the size of the reaction site and therefore an upper bound on the density of reaction sites. Successful elongation (for a given viscosity and delivered volume) was determined by the observation of bimodal distributions or bands on a gel (i.e., a band of successfully elongated product and a band of capped initiators from outside of the reaction sites, as illustrated in FIG. 9B). Results are shown in the following table, where “No” indicates no elongated initiators were detected and “Yes” indicates that elongated initiators were detected by the appearance of bimodal bands on a gel.

Number of Pulses (volume)

| Viscosity | 1 | 5 | 10 | 20 | 30 | 40 | 50 | 60 |
|---|---|---|---|---|---|---|---|---|
| 3.7 mPa · s | No | No | No\hat{ } | NTA | NT | NT | NT | — |
| 40 wt-%* | | | | | | | | |
| 2.5 mPa · s | NT | NT | NT | NT | NT | NT | NT | Yes |
| 30 wt-%* | | | | | | | | |
| 1.8 mPa · s | NT | NT | Yes | Yes | Yes | Yes | Yes | Yes |
| 20 wt-%* | | | | | | | | |
| 1.3 mPa · s | Yes | NT | Yes | Yes | Yes | Yes | Yes | Yes |
| 10 wt-%* | | | | | | | | |

*Viscosity of glycerol: water mixture at 21° C.
\hat{ }“NT” means conditions not tested.

The results show that for successful synthesis on small reaction sites (necessary for high densities) the viscosity of an ink must be low, e.g. less than 2 mPa·s for reaction sites formed using 2-3 μL droplets.

### Example 4

### Enzymatic Synthesis Cycles Using a Capping Step

In this example, the benefit of a capping step is demonstrated. The synthesis was performed essentially as described in Example 2 for each of two slides (i.e. synthesis took place on eight rows of three regions each), with the exceptions that the enzyme buffers contained 11% glycerol and the no-capping slide 2 was incubated for 5 min post-synthesis. On slide 1 a capping step (performed as described in Example 2) was included and on slide 2 the capping step was omitted. The results are shown in FIG. 9D where electropherograms (932 and 934) are shown side-by-side with separated products from rows of slide 1 (capping) and slide 2 (no capping), respectively. Lanes 1-8 on each electropherogram corresponds to rows 1-8 on each slide. Full length products of the syntheses are indicated by arrow (936). The benefit of the capping step is shown by the presence of intermediate length failure sequences (938) in the products from slide 2 and their absence in the product from slide 1.

### Example 5

### Synthesis in Ambient Air without Environmental Controls

In this example, a 5-cycle synthesis was performed on two slides as described above, except that synthesis took place in rows 1-6 instead of rows 3-8. The syntheses on the two slides were identical except that one was incubated in a humidity chamber during elongation (slide 1) and the slide was left out on the lab bench during elongation (slide 2). Humidity and temperature were measured with a Testo 175H1 data logger. The temperature and average relative humidity for slide 1 were 21° C. and about 80%, respectively, and for slide 2 the values were 21° C. and about 36%, respectively. After synthesis, products were cleaved, pooled and loaded onto gels as described above. The results are shown in FIG. 9E. They show that the difference in relative humidity has little impact on the resulting product yields.

### Example 6

### Dyes for Monitoring Reaction Site Geometry

In this example, various dyes were added to synthesis reagent inks to facilitate monitoring of reaction site geometry, including size, shape, overlap or coalescence with adjacent sites, and the like. The object of these experiments was to identify at least four different colored dyes that facilitated image processing while at the same time had no effect on synthesis. The following dyes were tested: Bromocresol Green, Bromocresol Purple. Chlorophenol Red, Methylene Blue and Xylenecyanol FF. The dyes were combined with the standard enzyme buffer (0.5 M cacodylate buffer pH 7.5, 1 mM CoCl2, 500 μM dTTP-ONH2, 20 μM4 H4 M57, 50 mM BOX aldehyde scavenger, 0.005% Tween 20) with 11 w % glycerol) with a final concentration of about 0.6 mg/mL (or between 1-2 mM depending on the dye). The enzyme buffers were spotted manually (10 uL droplets) on discrete regions of FAM labelled, photocleavable, DNA initiators (as described above). Each colored enzyme buffer was applied to 3 such regions, and enzyme buffer without dye was applied to 9 regions as controls. The slide was incubated for 5 min in a pipette tip box containing−50 mL of SSC buffer 1×. After incubation, the slide was washed for 3 min in deblocking buffer and 1 min in MQ H2O, 10 cycles of synthesis were then performed, after which synthesis products were cleaved (25 min incubation in PBS under UV illumination at 365 nm) and analyzed by gel electrophoresis. As shown in FIG. 9F, none of the dyes, except for Bromocresol Green, affected the synthesis.

### Definitions

Unless otherwise specifically defined herein, terms and symbols of nucleic acid chemistry, biochemistry, genetics, and molecular biology used herein follow those of standard treatises and texts in the field. e.g. Kornberg and Baker, DNA Replication. Second Edition (W.H. Freeman, New York, 1992); Lchninger. Biochemistry. Second Edition (Worth Publishers. New York, 1975); Strachan and Read, Human Molecular Genetics, Second Edition (Wiley-Liss, New York, 1999); Le. Recent Progress in Ink Jet Technologies II, chapter 1, pgs, 1-14 (1999); Zapka, editor, “Handbook of Industrial Inkjet Printing,” (Wiley-VCH, Weinheim. Germany).

“Functionally equivalent” in reference to amino acid positions in two or more different TdTs means (i) the amino acids at the respective positions play the same functional role in an activity of the TdTs, and (ii) the amino acids occur at homologous amino acid positions in the amino acid sequences of the respective TdTs. It is possible to identify positionally equivalent or homologous amino acid residues in the amino acid sequences of two or more different TdTs on the basis of sequence alignment and/or molecular modelling. In some embodiments, functionally equivalent amino acid positions belong to inefficiency motifs that are conserved among the amino acid sequences of TdTs of evolutionarily related species. e.g. genus, families, or the like. Examples of such conserved inefficiency motifs are described in Motca ct al, Biochim. Biophys. Acta. 1804(5): 1151-1166 (2010); Dclaruc ct al. EMBO J., 21: 427-439 (2002); and like references.

“Humectant” is any hygroscopic substance that attracts and retains moisture. Exemplary humectants include, but are not limited to, glycerol, alcohol sugars, ethylhexylglycerin, panthenol, sorbitol, xylitol, maltitol, propylene glycol, hexylene glycol, butylene glycol, sodium lactate, hyaluronic acid, polydextrose, or the like.

“Mutant” or “variant,” which are used interchangeably, refer to polypeptides derived from a natural or reference TdT polypeptide described herein, and comprising a modification or an alteration, i.e., a substitution, insertion, and/or deletion, at one or more positions. Variants may be obtained by various techniques well known in the art. In particular, examples of techniques for altering the DNA sequence encoding the wild-type protein, include, but are not limited to, site-directed mutagenesis, random mutagenesis, sequence shuffling and synthetic oligonucleotide construction. Mutagenesis activities consist in deleting, inserting or substituting one or several amino-acids in the sequence of a protein or in the case of the invention of a polymerase. The following terminology is used to designate a substitution: L238A denotes that amino acid residue (Leucine. L) at position 238 of a reference, or wild type, sequence is changed to an Alanine (A). A132V/I/M denotes that amino acid residue (Alanine, A) at position 132 of the parent sequence is substituted by one of the following amino acids: Valine (V), Isoleucine (I), or Methionine (M). The substitution can be a conservative or non-conservative substitution. Examples of conservative substitutions are within the groups of basic amino acids (arginine, lysine and histidine), acidic amino acids (glutamic acid and aspartic acid), polar amino acids (glutamine, asparagine and threonine), hydrophobic amino acids (methionine, leucine, isoleucine, cysteine and valine), aromatic amino acids (phenylalanine, tryptophan and tyrosine), and small amino acids (glycine, alanine and serine).

“Polynucleotide” or “oligonucleotide” are used interchangeably and each mean a linear polymer of nucleotide monomers or analogs thereof. Monomers making up polynucleotides and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Such monomers and their internucleosidic linkages may be naturally occurring or may be analogs thereof, e.g. naturally occurring or non-naturally occurring analogs. Non-naturally occurring analogs may include PNAs, phosphorothioate internucleosidic linkages, bases containing linking groups permitting the attachment of labels, such as fluorophores, or haptens, and the like. Whenever the use of an oligonucleotide or polynucleotide requires enzymatic processing, such as extension by a polymerase, ligation by a ligase, or the like, one of ordinary skill would understand that oligonucleotides or polynucleotides in those instances would not contain certain analogs of internucleosidic linkages, sugar moieties, or bases at any or some positions. Polynucleotides typically range in size from a few monomeric units, e.g. 5-40, when they are usually referred to as “oligonucleotides,” to several thousand monomeric units. Whenever a polynucleotide or oligonucleotide is represented by a sequence of letters (upper or lower case), such as “ATGCCTG,” it will be understood that the nucleotides are in 5′-+3′ order from left to right and that “A” denotes deoxyadenosine. “C” denotes deoxycytidine, “G” denotes deoxyguanosine, and “T” denotes thymidine, “I” denotes deoxyinosine, “U” denotes uridine, unless otherwise indicated or obvious from context. Unless otherwise noted the terminology and atom numbering conventions will follow those disclosed in Strachan and Read, Human Molecular Genetics 2 (Wiley-Liss, New York, 1999). Usually polynucleotides comprise the four natural nucleosides (e.g. deoxyadenosine, deoxycytidine, deoxyguanosine, deoxythymidine for DNA or their ribose counterparts for RNA) linked by phosphodiester linkages; however, they may also comprise non-natural nucleotide analogs. e.g. including modified bases, sugars, or internucleosidic linkages. It is clear to those skilled in the art that where an enzyme has specific oligonucleotide or polynucleotide substrate requirements for activity, e.g. single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al. Molecular Cloning, Second Edition (Cold Spring Harbor Laboratory, New York, 1989), and like references. Likewise, the oligonucleotide and polynucleotide may refer to either a single stranded form or a double stranded form (i.e. duplexes of an oligonucleotide or polynucleotide and its respective complement). It will be clear to one of ordinary skill which form or whether both forms are intended from the context of the terms usage.

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following references that are incorporated by reference: Dieffenbach, editor, PCR Primer: A Laboratory Manual, 2nd Edition (Cold Spring Harbor Press, New York, 2003).

“Sequence identity” refers to the number (or fraction, usually expressed as a percentage) of matches (e.g., identical amino acid residues) between two sequences, such as two polypeptide sequences or two polynucleotide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Sequences of similar lengths are preferably aligned using a global alignment algorithm (e.g. Needleman and Wunsch algorithm; Needleman and Wunsch. 1970) which aligns the sequences optimally over the entire length, while sequences of substantially different lengths are preferably aligned using a local alignment algorithm (e.g. Smith and Waterman algorithm (Smith and Waterman, 1981) or Altschul algorithm (Altschul et al., 1997; Altschul et al., 2005)). Alignment for purposes of determining percent amino acid sequence identity can be achieved in various ways that are within the skill in the art, for instance, using publicly available computer software available on internet web sites such as http://blast.ncbi.nlm.nih.gov/or ttp://www.ebi.ac.uk/Tools/emboss/. Those skilled in the art can determine appropriate parameters for measuring alignment, including any algorithm needed to achieve maximal alignment over the full length of the sequences being compared. For purposes herein, % amino acid sequence identity values refer to values generated using the pair wise sequence alignment program EMBOSS Needle, that creates an optimal global alignment of two sequences using the Needleman-Wunsch algorithm, wherein all search parameters are set to default values, i.e. Scoring matrix=BLOSUM62, Gap open=10, Gap extend=0.5. End gap penalty=false, End gap open=10 and End gap extend=0.5.

“Substitution” means that an amino acid residue is replaced by another amino acid residue. Preferably, the term “substitution” refers to the replacement of an amino acid residue by another selected from the naturally-occurring standard 20 amino acid residues, rare naturally occurring amino acid residues (e.g. hydroxyproline, hydroxylysine, allohydroxylysine, 6-N-methyllysine, N-ethylglycine, N-methylglycine, N-ethylasparagine, allo-isoleucine, N-methylisoleucine, N-methylvaline, pyroglutamate, aminobutyric acid, omithine, norleucine, norvaline), and non-naturally occurring amino acid residue, often made synthetically, (e.g. cyclohexyl-alanine). Preferably, the term “substitution” refers to the replacement of an amino acid residue by another selected from the naturally-occurring standard 20 amino acid residues. The sign “+” indicates a combination of substitutions. The amino acids are herein represented by their one-letter or three-letters code according to the following nomenclature: A: alanine (Ala); C: cysteine (Cys); D: aspartic acid (Asp); E: glutamic acid (Glu); F: phenylalanine (Phe); G: glycine (Gly); H: histidine (His); I: isoleucine (Tle); K: lysine (Lys); L: leucine (Leu); M: methionine (Met); N: asparagine (Asn); P: proline (Pro); Q: glutamine (Gin); R: arginine (Arg); S: serine (Ser); T: threonine (Thr); V: valine (Val); W: tryptophan (Trp) and Y: tyrosine (Tyr). In the present document, the following terminology is used to designate a substitution: L238A denotes that amino acid residue (Leucine, L) at position 238 of the parent sequence is changed to an Alanine (A). A132V/I/M denotes that amino acid residue (Alanine, A) at position 132 of the parent sequence is substituted by one of the following amino acids: Valine (V), Isoleucine (I), or Methionine (M). The substitution can be a conservative or non-conservative substitution. Examples of conservative substitutions are within the groups of basic amino acids (arginine, lysine and histidine), acidic amino acids (glutamic acid and aspartic acid), polar amino acids (glutamine, asparagine and threonine), hydrophobic amino acids (methionine, leucine, isoleucine, cysteine and valine), aromatic amino acids (phenylalanine, tryptophan and tyrosine), and small amino acids (glycine, alanine and serine).

1. A method of enzymatically synthesizing a plurality of polynucleotides each having a predetermined sequence at reaction sites on a planar substrate, the method comprising the steps of:
(a) providing a planar substrate having initiators at a plurality of reaction sites, wherein each initiator has a free 3′-hydroxyl and wherein each polynucleotide of the plurality is assigned to a reaction site for synthesis;
(b) dispensing through one or more inkjet pumps at least one droplet of at least one synthesis reagent to each reaction site of the plurality to perform a reaction cycle comprising the steps of (i) reacting under elongation conditions the initiator or elongated fragments having free 3′-O-hydroxyls with a 3′-O-protected nucleoside triphosphate and a template-free polymerase so that the initiator or elongated fragments are elongated by incorporation of a 3′-O-protected nucleoside triphosphate to form 3′-O-protected elongated fragments, and (ii) deprotecting the elongated fragments to form elongated fragments having free 3′-hydroxyls, wherein the synthesis reagent comprises a template-free polymerase, a 3′-O-protected nucleoside triphosphate, a mixture of a template-free polymerase and a 3′-O-protected nucleoside triphosphate, or a deprotection solution;
(c) repeating step (b) until the plurality of polynucleotides is synthesized.

2. The method of 1 further including a step of cleaving said plurality of polynucleotides from said reaction sites after said plurality are synthesized.

3. The method of claim 1 wherein said at least one synthesis reagent comprises a template-free polymerase.

4. The method of claim 3 wherein said at least one synthesis reagent is a printable terminal deoxynucleotidyl transferase (TdT) ink.

5. The method of claim 4 wherein said printable TdT ink further comprises a 3′-O-protected-2′-deoxynucleoside triphosphate.

6. (canceled)

7. The method of claim 1 wherein said at least one synthesis reagent is a printable nucleotide monomer ink.

8-10. (canceled)

11. The method of any one of claim 1 wherein each of said reaction sites are distinct and non-overlapping with other said reaction sites.

12. The method of claim 1 wherein said step of reacting includes incubating said reaction mixture for a predetermine duration.

13. The method of claim 1 wherein said reaction sites are imaged during said step of incubating.

14. The method of claim 1 wherein said step of dispensing includes at least one of said inkjet pumps delivering said droplets to at least one of said reaction sites in move-stop droplet delivery mode.

15-17. (canceled)

18. The method of claim 1 wherein each of said polynucleotides of said plurality is assigned to a different reaction site for synthesis.

19. A method of enzymatically synthesizing a plurality of polynucleotides each having a predetermined sequence at distinct reaction sites on a planar substrate, the method comprising the steps of:
(a) providing a planar substrate having initiators at a plurality of distinct reaction sites, wherein each initiator has a free 3′-hydroxyl and wherein each polynucleotide of the plurality is assigned to a different reaction site for synthesis;
(b) dispensing to each reaction site a buffer solution comprising a template-free polymerase;
(c) dispensing through one or more inkjet pumps at least one droplet of a buffer solution comprising a 3′-O-blocked-dATP, a 3′-O-blocked-dCTP, a 3′-O-blocked-dGTP, or a 3′-O-blocked-dTTP to each reaction site of the plurality, wherein the kind of 3′-O-blocked dNTP dispensed to a reaction site depends on the predetermined sequence of the polynucleotide assigned to the reaction site;
(d) incubating the template-free polymerase and 3′-O-blocked-dNTPs at each reaction site so that initiators or elongated fragments at the reaction site are elongated by incorporation of a 3′-O-blocked nucleoside triphosphate to form 3′-O-blocked elongated fragments;
(e) deblocking the elongated fragments to form elongated fragments having free 3′-hydroxyls by treating the planar support with a deblocking agent;
(f) repeating steps (b), (c), (d) and (e) until the plurality of polynucleotides is synthesized.

20. The method of claim 19 wherein said step of dispensing said template-free polymerase to each reaction site is carried out by dispensing through one or more inkjet pumps at least one droplet of said buffer solution comprising said template-free polymerase.

21. The method of claim 19 wherein said steps of dispensing to each reaction site said buffer comprising said template-free polymerase and said buffer solution comprising said 3′-O-blocked-dATP, said 3′-O-blocked-dCTP, said 3′-O-blocked-dGTP, or said 3′-O-blocked-dTTP are carried out by dispensing to each reaction site a buffer comprising a mixture of said template-free polymerase and said 3′-O-blocked-dATP, or said 3′-O-blocked-dCTP, or said 3′-O-blocked-dGTP, or said 3′-O-blocked-dTTP.

22. The method of claim 19 wherein said step of dispensing said template-free polymerase is accomplished by flowing said buffer comprising said template-free polymerase over all of said reaction sites.

23. (canceled)

24. (canceled)

25. A printable template-free polymerase ink, comprising: an aqueous solution comprising a template-free polymerase having a concentration of in a range of from 1.0 μM to 30 μM; wherein whenever the ink is printed to a substrate, printed droplets each have a volume in the range of 0.1 μL to 5 nL of the aqueous solution and wherein the ink is characterized by a viscosity in the range of about 1 centipoise to about 20 centipoise when viscosity is measured at room temperature; and a surface tension in the range of about 15 dynes/cm to about 50 dynes/cm when measured at room temperature.

26. The printable template-free polymerase ink of claim 25 further comprising a viscosity-modifying agent, a surface tension modifying agent or combination thereof.

27-29. (canceled)

30. The printable template-free polymerase ink of claim 25 for catalyzing a reaction between a 3′-O-amino-nucleoside triphosphate and a free 3′-hydroxyl of an initiator or an elongated fragment, the printable template-free polymerase ink further comprising an aldehyde scavenger.

31. (canceled)

32. The printable template-free polymerase ink of claim 25 wherein said viscosity is equivalent to a viscosity produced by glycerol in a concentration range of 5 percent (w/w) to 40 percent (w/w).

33. The printable template-free polymerase ink of claim 25 wherein said template-free polymerase is a terminal deoxynucleotidyltransferase (TdT) variant.
